(19) United States
(12) Patent Application Publication
(10) Pub. No.: US 2025/0041286 A1
(43) Pub. Date: Feb. 6, 2025

(54) CANCER TREATMENT USING KETOTIFEN IN COMBINATION WITH A CHECKPOINT INHIBITOR

(71) Applicants: Materia Therapeutics Inc., Las Vegas, NV (US); University of Cyprus, Aglantzia (CY)

(72) Inventors: John D. MARTIN, Nicosia (CY); Triantafyllos STYLIANOPOULOS, Nicosia (CY); Fotios MPEKRIS, Nicosia (CY); Myrofora PANAGI, Nicosia (CY); Chrysovalantis VOUTOURI, Nicosia (CY)

(21) Appl. No.: 18/717,876

(22) PCT Filed: Dec. 8, 2022
(86) PCT No.: PCT/US2022/081197

§ 371 Date: Jun. 7, 2024

Related U.S. Application Data
(60) Provisional application No. 63/287,872, filed on Dec. 9, 2021.

(51) Int. Cl. A61K 31/4535; A61K 31/704; A61K 39/00; A61P 35/00; C07K 16/28

(52) U.S. Cl. CPC A61K 31/4535; A61K 31/704; A61P 35/00; C07K 16/2818; A61K 2039/507

(57) ABSTRACT

The present invention is directed to a combination treatment using ketotifen and a checkpoint inhibitor that is effective in treating cancer or inhibiting the proliferation of tumor cells in a subject and/or that can initiate, enhance or prolong the immune response to tumor cells.

### CROSS-REFERENCE TO RELATED APPLICATIONS

This application claims the benefit of U.S. Provisional Application No. 63/287,872 filed Dec. 9, 2021, the contents of which are incorporated herein by reference.

### INCORPORATION BY REFERENCE OF SEQUENCE LISTING

The contents of the electronic sequence listing (211482000240SEQLIST.xml; Size: 23,937 bytes; and Date of Creation: Dec. 8, 2022) is herein incorporated by reference in its entirety.

### FIELD OF THE INVENTION

The present invention discloses a combination treatment using ketotifen and a checkpoint inhibitor that is effective in treating cancer or inhibiting the proliferation of tumor cells in a subject and/or that can initiate, enhance or prolong the immune response to tumor cells.

### BACKGROUND

Efficacy of cancer immunotherapy depends, in part, on whether T cells can traffic to tumors and migrate to a location adjacent to malignant cells to recognize and kill them. One barrier to T cell homing is the tumor blood vessel wall, which inhibits T cell attachment and transmigration through the endothelin B receptor, but antagonizing this receptor has not yet led to a clinically approved drug. One reason could be hypoperfusion in tumors, which could limit the surface area of perfused blood vessels for anti-tumor T cells to attach. If collapsed tumor blood vessels could be decompressed and reperfused by alleviating mechanical compression (i.e. solid stress), endothelin B receptor antagonism could increase the efficacy of cancer immunotherapy.

Ketotifen is a cycloheptathiophene derivative drug initially marketed as an inhibitor of anaphylaxis. Ketotifen selectively blocks histamine (H1) receptors and suppresses symptoms associated with histamine release. Ketotifen has also been reported to suppress the activity of mast cells.

Immune checkpoints, which act as the off-switch on the T cells of the immune system, have been investigated to reinstate the immune response with targeted agents, thus indirectly treating cancer by activating the body's immune system.

International applications WO2002086083, WO2004004771, WO2004056875, WO2006121168, WO2008156712, WO2010077634, WO2011066389, WO2014055897 and WO2014100079 report PD-1, PD-L1 inhibitory antibodies and/or methods of identifying such antibodies. Further, U.S. patents such as U.S. Pat. Nos. 8,735,553 and 8,168,757 report PD-1 or PD-L1 inhibitory antibodies and/or fusion proteins. The disclosures of WO2002086083, WO2004004771, WO2004056875, WO2006121168, WO2008156712, WO2010077634, WO2011066389, WO2014055897 and WO2014100079, as well as U.S. Pat. Nos. 8,735,553 and 8,168,757, are incorporated herein by reference in their entirety.

Moreover, International applications WO2011161699, WO2012168944, WO2013144704, WO2013132317 and WO 2016044900 report peptides or peptidomimetic compounds which are capable of suppressing and/or inhibiting the programmed cell death 1 (PD-1) signaling pathway. The disclosures of WO2011161699, WO2012168944, WO2013144704, WO2013132317 and WO 2016044900 are incorporated herein by reference in their entirety.

Furthermore, International applications WO 2016142852, WO 2016142894, WO 2016142886, WO 2016142835 and WO 2016142833 report small molecule compounds which are capable of suppressing and/or inhibiting the programmed cell death 1 (PD-1) signaling pathway and/or treating disorders by inhibiting an immunosuppressive signal induced by PD-1, PD-L1 or PD-L2. The disclosures of WO 2016142852, WO 2016142894, WO 2016142886, WO 2016142835 and WO 2016142833 are incorporated herein by reference in their entirety.

Recently, ipilimumab (Yervoy®), a monoclonal antibody that targets cytotoxic T-lymphocyte-associated antigen 4 (CTLA-4) and nivolumab (Opdivo®), a monoclonal antibody that targets the programmed cell death protein 1 pathway (PD-1) on the surface of T-cells, have been approved by the U.S. Food and Drug Administration for the treatment of advanced melanoma, advanced renal cell carcinoma, and non-small cell lung cancer. Current checkpoint inhibitor therapies, however, are effective at treating cancer in a relatively small population of cancer subject population, which is in part due to pre-existing immune activation and presence of the inhibitory receptors. Although immune checkpoint blockade (ICB) with checkpoint inhibitors has revolutionized treatment for many types of solid tumors, it is now estimated to benefit less than 20% of cancer patients. Increasing the fraction of patients responding and the length of their response is an urgent unmet clinical need. Accordingly, there is a need to develop methods and combination therapies to initiate or enhance the effectiveness of the checkpoint inhibitors in both the nonresponding subject population and the responding subject population.

Anti-tumor T cells must circulate into tumors through blood vessels, bind the endothelium, and pass across the vessel wall and migrate through cancer-associated fibroblasts (CAFs) and extracellular matrix (ECM) before encountering cancer cells. However, because up to 80% of intratumoral blood vessels lack perfusion, the area of vessel wall for T cells to migrate across is limited.

Compressed blood vessels impair blood flow and oxygen delivery to tumors, resulting in increased hypoxia in the tumors and resistance to immunotherapy through numerous mechanisms. Strategies that decompress vessels potentiate the efficacy of ICB in ICB-resistant mouse models of metastatic breast cancer. If there was a way to decompress tumor vessels while also facilitating vessel adhesion and transmigration of T cells into the tumor parenchyma, the fraction of cancer patients that respond to ICB could increase.

All references cited herein, including patent applications, patent publications, and scientific literature, are herein incorporated by reference in their entirety, as if each individual reference were specifically and individually indicated to be incorporated by reference.

### SUMMARY

Provided herein are methods for treating a solid tumor in a subject in need thereof comprising administering to the subject an effective amount of ketotifen, or a pharmaceutically acceptable salt thereof, in combination with a checkpoint inhibitor. Also provided herein are methods for initiating, enhancing or prolonging the effects of a checkpoint inhibitor, or enabling a subject to respond to a checkpoint inhibitor in a subject in need thereof comprising administering to the subject an effective amount of ketotifen, or a pharmaceutically acceptable salt thereof, in combination with a checkpoint inhibitor, wherein the subject has a solid tumor. Also provided herein are methods for potentiating the effects of a checkpoint inhibitor in a subject in need thereof comprising administering to the subject an effective amount of ketotifen, or a pharmaceutically acceptable salt thereof, in combination with a checkpoint inhibitor, wherein the subject has a solid tumor. Also provided herein are methods for increasing blood flow of a solid tumor in a subject comprising administering to the subject an effective amount of ketotifen, or a pharmaceutically acceptable salt thereof, in combination with a checkpoint inhibitor, wherein increasing blood flow of the solid tumor enhances the effect of the checkpoint inhibitor. In some embodiments, blood flow is measured using ultrasound-based blood flow measurements or using histological techniques to measure hypoxia. Also provided herein are methods of improving the delivery or efficacy of a checkpoint inhibitor in a subject comprising administering an effective amount of ketotifen, or a pharmaceutically acceptable salt thereof, in combination with the checkpoint inhibitor, wherein the subject has a solid tumor, thereby improving the delivery or efficacy of the therapy in the subject. In some embodiments, administering ketotifen, or pharmaceutically acceptable salt thereof, increases the number of anti-tumor T cells that colocalize with the solid tumor. In some embodiments, administering ketotifen, or a pharmaceutically acceptable salt thereof, reduces the tissue stiffness of the solid tumor. In some embodiments, the tissue stiffness of the solid tumor is measured using ultrasound elastography. In some embodiments, administering ketotifen, or pharmaceutically acceptable salt thereof, decreases the levels of an extracellular matrix protein in the solid tumor. In some embodiments, the extracellular matrix protein is collagen I or hyaluronan binding protein (HABP). In some embodiments, administering ketotifen, or pharmaceutically acceptable salt thereof, reduces hypoxia in the solid tumor. In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof. In some embodiments, the checkpoint inhibitor is a CTLA-4, PD-L1, PD-L2, or PD-1 inhibitor. In some embodiments, the checkpoint inhibitor is an anti-PD-1 antibody, anti-PD-L1 antibody, or anti-CTLA4 antibody. In some embodiments, the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor is a combination of an anti-PD-1 antibody and an anti-CTLA-4 antibody. In some embodiments, the ketotifen, or pharmaceutically acceptable salt thereof, is administered to the subject once per day. In some embodiments, the ketotifen, or pharmaceutically acceptable salt thereof, is administered to the subject twice per day. In some embodiments, the ketotifen, or pharmaceutically acceptable salt thereof, is administered to the subject at a dose from about 0.01 mg/kg to about 5 mg/kg. In some embodiments, the ketotifen, or pharmaceutically acceptable salt thereof, is administered to the subject at a dose from about 100 mg to about 1200 mg. In some embodiments, the ketotifen, or pharmaceutically acceptable salt thereof, is administered to the subject at a dose from about 125 mg to about 500 mg. In some embodiments, the ketotifen, or pharmaceutically acceptable salt thereof, is administered to the subject at a dose of about 125 mg. In some embodiments, the ketotifen, or pharmaceutically acceptable salt thereof, is administered to the subject at a dose of about 500 mg. In some embodiments, the ketotifen, or pharmaceutically acceptable salt thereof, is administered to the subject prior to the subject being administered the checkpoint inhibitor. In some embodiments, the ketotifen, or pharmaceutically acceptable salt thereof, is administered to the subject beginning at least 1 day prior to the subject being administered the checkpoint inhibitor. In some embodiments, the ketotifen, or pharmaceutically acceptable salt thereof, is administered to the subject beginning at least 2 days prior to the subject being administered the checkpoint inhibitor. In some embodiments, the ketotifen, or pharmaceutically acceptable salt thereof, is administered to the subject beginning at least 3 days prior to the subject being administered the checkpoint inhibitor. In some embodiments, the ketotifen, or pharmaceutically acceptable salt thereof, is administered to the subject beginning at least 5 days prior to the subject being administered the checkpoint inhibitor. In some embodiments, the administration of ketotifen, or pharmaceutically acceptable salt thereof, to the subject is maintained for at least a portion of the time the subject is administered the checkpoint inhibitor. In some embodiments, the administration of ketotifen, or pharmaceutically acceptable salt thereof, to the subject is maintained for the entire period of time the subject is administered the checkpoint inhibitor. In some embodiments, one or more therapeutic effects in the subject is improved after administration of the ketotifen, or pharmaceutically acceptable salt thereof, and the checkpoint inhibitor relative to a baseline. In some embodiments, the one or more therapeutic effects is selected from the group consisting of: size of a tumor derived from the cancer, objective response rate, duration of response, time to response, progression free survival and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the ketotifen, or pharmaceutically acceptable salt thereof, and the checkpoint inhibitor. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor. In some embodiments, the solid tumor is selected from the group consisting of mesothelioma, breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma. In some embodiments, the solid tumor is a sarcoma. In some embodiments, the sarcoma is osteosarcoma or fibrosarcoma. In some embodiments, the subject is a human. In some embodiments of any of the preceding methods, the method further comprises administering an additional chemotherapeutic agent. In some embodiments, the additional chemotherapeutic agent is doxorubicin or an analogue or derivative thereof.

Also provided herein is a kit comprising: (a) an effective amount of ketotifen, or a pharmaceutically acceptable salt thereof; (b) an effective amount of a checkpoint inhibitor; and (c) instructions for using the ketotifen, or a pharmaceutically acceptable salt thereof and the checkpoint inhibitor according to any of the methods described herein.

Also provided herein are methods of determining an effective amount of ketotifen in a subject with a solid tumor comprising: (a) measuring the blood flow and/or stiffness of the solid tumor; (b) administering to the subject an effective amount of ketotifen; and (c) measuring the blood flow and/or stiffness of the solid tumor after administering the ketotifen, wherein an increase in blood flow and/or a decrease in stiffness following administration of the ketotifen to the subject indicates that the amount administered was an effective amount. Also provided herein are methods for treating a solid tumor in a subject in need thereof comprising: (a) measuring the blood flow and/or stiffness of the solid tumor; (b) administering to the subject an effective amount of ketotifen; (c) measuring the blood flow and/or stiffness of the solid tumor after administering the ketotifen; and (d) administering a chemotherapeutic agent if the blood flow of the solid tumor is increased and/or the stiffness of the solid tumor is decreased after administering the ketotifen. Also provided herein are methods for treating a solid tumor in a subject in need thereof comprising: (a) measuring the blood flow and/or stiffness of the solid tumor; (b) administering to the subject an effective amount of ketotifen; (c) measuring the blood flow and/or stiffness of the solid tumor after administering the ketotifen; (d) determining that the subject is responsive to a chemotherapeutic agent based on an increase in the blood flow of the solid tumor or a decrease in the stiffness of the solid tumor after administering the ketotifen; and (e) administering the chemotherapeutic agent to the subject who has been determined to be responsive to the chemotherapeutic agent based on the increase in the blood flow of the solid tumor or the decrease in the stiffness of the solid tumor after administering the ketotifen. Also provided herein are methods for predicting response to treatment with a chemotherapeutic agent comprising: (a) measuring the blood flow and/or stiffness of the solid tumor; (b) administering to the subject an effective amount of ketotifen; (c) measuring the blood flow and/or stiffness of the solid tumor after administering the ketotifen, wherein an increase in the blood flow of the solid tumor or a decrease in the stiffness of the solid tumor after administering the ketotifen indicates that the subject is likely to respond to treatment with the chemotherapeutic agent. In some embodiments, the effective amount of ketotifen is determined by measuring the change in blood flow and/or stiffness of the solid tumor following administration of the ketotifen to the subject, wherein an increase in blood flow and/or a decrease in stiffness following administration of the ketotifen to the subject indicates that the amount administered was an effective amount. In some embodiments, the method comprises measuring the blood flow of the solid tumor and the blood flow of the solid tumor is increased after administering the ketotifen. In some embodiments, the method comprises measuring the stiffness of the solid tumor and the stiffness of the solid tumor is decreased after administering the ketotifen. In some embodiments, the ketotifen is administered for at least 1 day, at least 2 days, at least 3 days, at least 4 days, or at least 5 days prior to the administration of the chemotherapeutic agent. In some embodiments, the ketotifen is administered at a dose that increases the blood flow of the solid tumor and/or decreases the stiffness of the solid tumor. In some embodiments, blood flow and/or stiffness of the solid tumor is measured using ultrasound. In some embodiments, blood flow of the solid tumor is measured using histological techniques to measure hypoxia. In some embodiments, the chemotherapeutic agent is a checkpoint inhibitor. In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof. In some embodiments, the checkpoint inhibitor is a CTLA-4, PD-L1, PD-L2, or PD-1 inhibitor. In some embodiments, the checkpoint inhibitor is an anti-PD-1 antibody, anti-PD-L1 antibody, or anti-CTLA4 antibody. In some embodiments, the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor is a combination of an anti-PD-1 antibody and an anti-CTLA-4 antibody. In some embodiments, the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma. In some embodiments, the solid tumor is a sarcoma. In some embodiments, the sarcoma is osteosarcoma or fibrosarcoma. In some embodiments, the subject is a human. In some embodiments of the preceding methods, the chemotherapeutic agent is doxorubicin or an analogue or derivative thereof.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

Representative embodiments of the invention are disclosed by reference to the following figures. It should be understood that the embodiments depicted are not limited to the precise details shown.

FIG. 1A shows representative immunofluorescence images of MCA205 tumor model paraffin sections stained for pimonidazole adducts following pimonidazole hydrochloride injection; scale bar=0.2 mm (top two rows DAPI stain; bottom two rows corresponding hypoxia stains). FIGS. 1B-1C show the quantification of hypoxia area fraction normalized to DAPI stain of MCA205 (FIG. 1B) and K7M2wt (FIG. 1C) tumors (n=5 mice per group, N=3-5 image fields per mouse). All data are expressed as mean±standard error of the mean. Statistical analyses were performed by comparing the treated groups with the control *, and the 10 mg/kg with all other treatment groups **, with p≤0.05. FIG. 1D shows the quantification of mRNA expression levels of IFN-γ and VEGF in untreated and ketotifen (10 mg/kg) treated MCA205 tumors using the 2^{\hat{ }-ΔΔCT} method (3 biological×3 technical replicates were used). All data are expressed as mean±standard error of the mean. Statistical analyses were performed by comparing the treated groups with the control *, and the 10 mg/kg with all other treatment groups **, with p≤0.05. FIG. 1E shows IFP levels in untreated and daily ketotifen treated mice for 7 days (n=7 mice per treatment group). All data are expressed as mean±standard error of the mean. Statistical analyses were performed by comparing the treated groups with the control *, and the 10 mg/kg with all other treatment groups **, with p≤0.05.

FIG. 2A shows growth curves of MCA205 tumors after administration of daily ketotifen at various doses (1, 5, 10 and 25 mg/kg) compared to control. FIG. 2B shows growth curves of K7M2wt tumors after administration of daily ketotifen at 10 mg/kg dose compared to control. FIGS. 2C-2D show measurements of MCA205 (FIG. 2C) and K7M2wt (FIG. 2D) tumor mass following completion of treatment protocol. All data are expressed as mean±standard error of the mean (n=5-7 mice per treatment group). FIG. 2E shows exemplary Western blot against the CD117 protein, with 3-actin as a loading control (n=4 mice per group).

FIGS. 3A-3F show mouse body, thymus and spleen weight at the conclusion of study of MCA205 (FIGS. 3A-3C) and K7M2wt (FIGS. 3D-3F) tumors treated with control or the indicated doses of ketotifen. All data are expressed as mean±standard error of the mean (n=5-7 mice per treatment group). Statistical analyses were performed by comparing the treated groups with the control * and the 10 mg/kg group, p≤0.05.

FIG. 4A shows an exemplary study treatment protocol for the murine MCA205 tumor model with an exemplary dose of 10 mg/kg daily ketotifen treatment. FIG. 4B and FIG. 4C show longitudinal measurements of tissue-level macroscopic Young's modulus in MCA205 (FIG. 4B) and K7M2wt (FIG. 4C) tumors treated as indicated, as assessed using ultrasound elastography (n=4 mice per group, N=2 image fields per mouse for ultrasound measurements). FIGS. 4D-4E shows vascular perfusion in MCA205 tumors treated as indicated at 3 days (FIG. 4D) and 7 days (FIG. 4E) post-ketotifen treatment. All data are expressed as mean±standard error of the mean (n=4 mice per treatment group). Statistical analyses were performed by comparing the treated groups with the control * and the 10 mg/kg group, p≤0.05. FIGS. 4F-4G show functional perfusion in K7M2wt tumors treated as indicated at 3 days (FIG. 4F) and 7 days (FIG. 4G) post-treatment. All data are expressed as mean±standard error of the mean (n=4 mice per treatment group). Statistical analyses were performed by comparing the treated groups with the control * and the 10 mg/kg group, p≤0.05.

FIG. 5A and FIG. 5B shows representative bright field images of picrosirius red staining in MCA205 (FIG. 5A) and K7M2wt (FIG. 5B) paraffin tumor sections (n=4 mice per group; N=5-6 image fields per mouse). FIG. 5C and FIG. 5D show the quantification of the area positive for Picrosirius red staining in fibrosarcoma MCA205 tumors (FIG. 5C) and osteosarcoma K7M2wt tumors (FIG. 5D) normalized to the control group. All data are expressed as mean±standard error of the mean (for histological analysis, n=4 mice per group, N=5-6 image fields per mouse). Statistical analyses were performed by comparing the treated groups with the control * and the 10 mg/kg with all other treatment groups **, p≤0.05. FIGS. 5E-5F show representative immunofluorescence images of Ki-67 proliferation marker and α-SMA in fibrosarcoma tumors, scale bar=0.2 mm (FIG. 5E; top two rows are α-SMA and bottom two rows are corresponding Ki67 stain) after treatment with control or indicated ketotifen doses; also shown is quantification of cancer associated fibroblasts (CAFs) positive for both the α-SMA and Ki-67 markers, normalized to total α-SMA staining (FIG. 5F) after treatment with control or the indicated doses of ketotifen. All data are expressed as mean±standard error of the mean (for histological analysis, n=4 mice per group, N=5-6 image fields per mouse). FIG. 5G shows quantification of mRNA expression levels of Col1A1, CTGF, ACTA2, HAS2 and HAS3 in untreated (control) and ketotifen (at 10 mg/kg) treated MCA205 tumors using the 2^{\hat{ }-ΔΔCT} method (3 biological×3 technical replicates were used). All data are expressed as mean±standard error of the mean. Statistical analyses were performed by comparing the treated groups with the control * and the 10 mg/kg with all other treatment groups **, p≤0.05. FIGS. 5H-5I show quantification of the fraction of area positive for α-SMA staining (FIG. 5H) and quantification of the fraction of area positive for Ki-67 staining (FIG. 5I) in MCA205 tumors after treatment with control or the indicated doses of ketotifen. All data are expressed as mean±standard error of the mean (for histological analysis, n=4 mice per group, N=5-6 image fields per mouse). FIGS. 5J-5L show representative immunofluorescence images of MCA205 paraffin sections stained with anti-HABP1, scale bar=0.2 mm (FIG. 5J; top two rows DAPI stain and bottom two rows corresponding HABP1 stain), quantification of the fraction of area positive for HABP1 staining in MCA205 tumors (FIG. 5K), and quantification of solid stress measured by the amount of length of the tumor opening after cutting the tissue (n=4 mice per group) (FIG. 5L), each after treatment with control or indicated doses of ketotifen. All data are expressed as mean±standard error of the mean (for histological analysis, n=4 mice per group, N=5-6 image fields per mouse). Statistical analyses were performed by comparing the treated groups with the control * and the 10 mg/kg with all other treatment groups **, p≤0.05.

FIG. 6A shows representative immunofluorescence images of Ki-67 proliferation marker (bottom row) and α-SMA (top row; top and bottom rows show same field) in K7M2wt tumors after treatment with control or indicated dose of ketotifen, scale bar=0.2 mm. FIG. 6B shows the quantification of cancer associated fibroblasts (CAFs) positive for both the α-SMA and Ki-67 markers, normalized to total α-SMA staining (n=4 mice per group, N=5-6 image fields per mouse) after treatment with control or indicated dose of ketotifen. FIG. 6C shows the quantification of the fraction of area positive for α-SMA staining after treatment with ketotifen compared with control. FIG. 6D shows the quantification of the fraction of area positive for Ki-67 staining in K7M2wt tumors after treatment with control or indicated dose of ketotifen. FIG. 6E shows representative immunofluorescence images of K7M2wt paraffin sections stained with anti-HABP1 (top row DAPI stain; bottom row corresponding HABP1 stain), scale bar=0.2 mm. FIG. 6F shows the quantification of the fraction of area positive for HABP1 staining (n=4 mice per group, N=5-6 image fields per mouse). All data are expressed as mean±standard error of the mean. Statistical analyses were performed by comparing the treated groups with the control * and the 10 mg/kg with all other treatment groups **, p≤0.05.

FIGS. 7A-7B show quantification of mRNA expression levels of Col1A1, CTGF, ACTA2, HAS2, HAS3, Col4, and TGFB in untreated and ketotifen (10 mg/kg) treated MCA205 tumors (FIG. 7A) and quantification of mRNA expression levels of Col1A1, CTGF, ACTA2, HAS2, HAS3, and Col4 in untreated and ketotifen (10 mg/kg) treated K7M2wt tumors (FIG. 7B) using the 2^{\hat{ }-ΔΔCT} method (3 biological×3 technical replicates were used). All data are expressed as mean±standard error of the mean. Statistical analyses were performed by comparing the treated groups with the control * and the 10 mg/kg with all other treatment groups **, p≤0.05.

FIG. 8A shows an exemplary study treatment protocol for the MCA205 tumor model used in FIGS. 8B-8H. FIGS. 8B-8C show relative growth curves of MCA205 tumors treated as indicated (n=10 mice per group) (FIG. 8B) and K7M2wt tumors treated as indicated (n=6 mice per group) (FIG. 8C). The symbol (**) indicates P<0.05 as determined by t-test, by comparing the ketotifen-doxorubicin-anti-PD-L1 group with all other treatment groups. FIG. 8D shows the correlation of elastic Young's modulus after daily ketotifen for 4 days in MCA205 tumors (n=5 mice per group), that were treated either with a-PD-L1 or doxorubicin alone, or with ketotifen combination and ketotifen-doxorubicin-anti-PD-L1 combination therapy to the relative tumor growth recorded by completion of the study, day 16 (R²=0.734, p<0.0001). FIG. 8E shows the correlation of elastic Young's modulus after daily ketotifen for 4 days in K7M2wt tumors (n=5 mice per group), that were treated either with anti-PD-L1 or doxorubicin alone, or with ketotifen combination and ketotifen-doxorubicin-anti-PD-L1 combination therapy to the relative tumor growth recorded by completion of study, day 33 (R²=0.857, p<0.0001). FIG. 8F shows the effect on tumor mass of MCA205 tumors (n=5 mice per group) at day 16 post treatment with ketotifen alone, anti-PD-L1 alone, doxorubicin alone, ketotifen and doxorubicin combination, doxorubicin and anti-PD-L1 combination, or ketotifen-doxorubicin-anti-PD-L1 combination therapy. FIG. 8G shows individual tumor growth curves of the surviving MCA205 bearing mice re-challenged with MCA205 tumor cells versus control mice naïve to MCA205 cancer cells inoculated on day 0 (n=5 mice per group) with the indicated treatments. FIG. 8H shows the functional perfusion area in MCA205 tumors (n=5 mice per group) after 4 days of treatment with the indicated treatments.

FIG. 9A shows the percentage of the total CD3⁺ T cells amongst CD45⁺ lymphocytes in whole tumor tissue of MCA205 tumor models treated as indicated. FIG. 9B shows the ratio of cytotoxic CD3⁺CD8⁺ T cells to CD3⁺CD4⁺CD25^{hi}CD127^{lo}Foxp3⁺ T regs (n=5 mice per group) in MCA205 tumors after the indicated treatments. FIG. 9C shows representative images of immunofluorescence (IF) staining of CD8 (bottom two rows) and Ki67 (top two rows, which correspond to same cells as bottom two rows) in K7M2wt paraffin embedded tissue sections, scale bar=0.1 mm. FIG. 9D shows corresponding quantification of proliferative CD8⁺ T cell fraction as the ratio of area that is double positive for CD8 and Ki67 staining to the area that is positive for total CD8 staining (n=4 mice per group, N=4-5 image fields per mouse) after treatment with the indicated therapies. FIG. 9E shows representative images of IF staining of CD31 (endothelial marker, top two rows) and CD3 (bottom two rows, which correspond to same image fields as top rows) in K7M2wt frozen tissue sections, scale bar=0.1 mm. FIG. 9F shows corresponding quantification of colocalization between CD3⁺ T cells and CD31⁺ endothelial cells (n=4 mice per group, N=4-5 image fields per mouse) after the indicated treatments. All data are expressed as mean±standard error of the mean. Statistical analyses were performed by comparing the treated groups with the control * and the ketotifen-doxorubicin-anti-PD-L1 with all other treatment groups **, p<0.05.

FIG. 10A shows representative IF images of K7M2wt paraffin embedded sections stained for pimonidazole adducts after pimonidazole hydrochloride injection, scale bar=0.2 mm (top two rows DAPI stain; bottom two rows corresponding hypoxia stain; top and bottom corresponding panels are the same image fields). FIG. 10B shows corresponding quantification of hypoxia area fraction normalized to DAPI stain of K7M2wt tumors (n=4 mice per treatment, N=4-5 image fields per mouse). FIG. 10C shows the relative mRNA levels of genes associated with immune cell adhesion on endothelial wall, assessed by RT-qPCR and quantified using the 2^{\hat{ }-ΔΔCT} method, in untreated (control) and 10 mg/kg ketotifen treated MCA205 tumors (3 biological×3 technical replicates were used). FIGS. 10D-10E show the quantification of the area positive for CD3 (FIG. 10D) and CD31 (FIG. 10E) staining in K7M2wt tumor sections after indicated treatments (n=4 mice per group, N=4-5 image fields per mouse). All data are expressed as mean±standard error of the mean. Statistical analyses were performed by comparing the treated groups with the control * and the ketotifen-doxorubicin-a-PD-L1 with all other treatment groups **, p<0.05.

FIGS. 11A-11B show flow cytometry analysis and gating design for analysis of MCA205 tumors. FIG. 11A shows flow cytometric analysis of myeloid cells of MCA205 tumors after indicated treatments. The myeloid gate was used on FSC/SSC, the dead cells were excluded, and gated on CD45⁺ cells (FIG. 11B). CD11b and Gr-1 (Ly6G⁺ Ly6C⁺) antibodies were used to distinguish the myeloid cell population from monocytes and MDSCs were identified by the high expression of both CD11b and Gr-1 proteins. All data are expressed as mean±standard error of the mean. Statistical analyses were performed by comparing the treated groups with the control *, p≤0.05.

FIG. 12 shows the gating strategy for flow cytometric analysis of tumor-infiltrating lymphocytes. Doublet/aggregated events were gated out using side scatter area versus side scatter width. After gating on singlets, live cells were identified using the L/D stain and next used the lymphocyte gate (CD45⁺) to filter out non-lymphocytic cells. CD3⁺ T cells were stained with CD4 and CD8 antibodies. After gating on CD4⁺ cells, the cells expressing low levels of CD127 and high levels of CD25 (CD127^{lo}CD25^{hi}) were identified. This population was gated on to identify the percentage of cells that are T regs expressing Foxp3⁺.

FIG. 13A shows a co-culture assay in which murine MC/9 MCs and NIH3T3 fibroblasts were separated by a transwell chamber with micropores that only allowed chemical communication. FIG. 13B shows the effect on mast cell degranulation by compound C48/80 (C48/80), ketotifen, and TGFβ. FIG. 13C shows immunofluorescence staining of αSMA (bottom row) and collagen I (top row) in fibroblasts after indicated treatments. FIG. 13D shows quantification of collagen I and FIG. 13E shows quantification of αSMA after the indicated treatments, as normalized to DAPI nuclear staining. Statistical analyses were performed by comparing means between two independent groups using the ordinary one-way ANOVA test.

FIG. 14A shows quantification of open lumen fraction in MCA205 tumors based on CD31 image analysis after treatment with control or indicated ketotifen doses. FIG. 14B shows representative immunofluorescence images of NG2 pericyte marker (middle two rows) and CD31 endothelial cell marker (top two rows) of MCA205 tumors upon treatment with different ketotifen concentrations (colocalized NG2/CD31 signal in bottom two rows, i.e., overlapping staining). FIG. 14C shows quantification of vessel pericyte coverage as indicated by CD31 and NG2 overlapping staining normalized to total CD31⁺ staining. FIG. 14D shows quantification of area fraction positive for CD31 staining (vessels) (n=5 mice per group, N=3-5 image fields per mouse).

FIG. 15A shows concentration-dependent activation of MC/9 cells by C48/80. FIG. 15B shows inhibitory effect of different concentrations of ketotifen on mast cell degranulation induced by C48/80. FIG. 15C shows NIH3T3 cell viability assessment in the presence of ketotifen or control for 2 or 24 hours.

FIG. 16 shows soft tissue sarcomas are infiltrated by mast cells. Immunofluorescence staining for CD117 (c-Kit) mast cell marker of human tissue arrays of patients with different sarcoma types (top row DAPI stain; bottom row corresponding CD117 stain).

FIG. 17A shows representative immunofluorescence images of mast cell tryptase from MCA205 tumors treated with control or the indicated doses of ketotifen (top two rows are DAPI nuclear stain; bottom two rows are corresponding tryptase stain). Scale bar is 0.05 mm. FIG. 17B and FIG. 17C show quantification of area positive for tryptase normalized to DAPI staining for MCA205 (FIG. 17B) and K7M2wt (FIG. 17C) tumors treated with control or indicated doses of ketotifen.

FIG. 18A shows normalized perfused area with respect to the total tumor area at the time of peak intensity for tumors after treatment with control or the indicated dose of ketotifen in the MCA205 tumor model. FIG. 18B shows normalized perfused area with respect to the total tumor area at the time of peak intensity for tumors after completion of treatment protocol as indicated in FIG. 4A (n=4-5) in the K7M2wt tumor model.

FIG. 19A shows average elastic modulus of the tumor micro-scale measured with atomic force microscopy (AFM) in K7M2wt tumors (n=3 mice per group, N=10-15 different 20×20 μm² force maps, 16×16 point grids). FIG. 19B shows representative AFM elastic modulus distributions/histograms of MCA205 (top left and bottom left) and K7M2wt (top right and bottom right) tumors after control or ketotifen treatment.

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

Compositions or methods “comprising” one or more recited elements may include other elements not specifically recited. For example, a composition that comprises antibody may contain the antibody alone or in combination with other ingredients.

It is understood that aspects and embodiments of the invention described herein include “comprising,” “consisting,” and “consisting essentially of” aspects and embodiments.

Designation of a range of values includes all integers within or defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “weight-based dose”, as referred to herein, means that a dose administered to a subject is calculated based on the weight of the subject. For example, when a subject with 60 kg body weight requires 2.0 mg/kg of ketotifen or a checkpoint inhibitor, one can calculate and use the appropriate amount of the ketotifen or checkpoint inhibitor (i.e., 120 mg) for administration to said subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., ketotifen and/or checkpoint inhibitor). For example, a subject with 60 kg body weight and a subject with 100 kg body weight would receive the same dose of ketotifen or checkpoint inhibitor.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

“Administering” or “administration” refer to the physical introduction of a therapeutic agent to a subject, using any of the various methods and delivery systems known to those skilled in the art. Exemplary routes of administration for the ketotifen and/or checkpoint inhibitor include enteral routes of administration and intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, MD, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the LIV1-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-LIV1 IgG1 antibody of the invention mediates equal or increased ADCC relative to a parental antibody and/or relative to an anti-LIV1 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-LIV1 IgG1 antibody of the invention mediates equal or increased ADCP relative to a parental antibody and/or relative to an anti-LIV1 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

As used herein, “subtherapeutic dose” means a dose of a therapeutic compound (e.g., ketotifen or a checkpoint inhibitor) that is lower than the usual or typical dose of the therapeutic compound when administered alone for the treatment of a hyperproliferative disease (e.g., cancer) and/or, for ketotifen, that is lower than the usual or typical dose used to treat its indicated disease (i.e. pulmonary hypertension).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); checkpoint inhibitors (e.g. inhibitors of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, or B-7 family ligands); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rlL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

The terms “baseline” or “baseline value” used interchangeably herein can refer to a measurement or characterization of a symptom before the administration of the therapy (e.g., ketotifen, or a pharmaceutically acceptable salt thereof as described herein and/or a checkpoint inhibitor as described herein) or at the beginning of administration of the therapy. The baseline value can be compared to a reference value in order to determine the reduction or improvement of a symptom of a disease, such as a cancer. The terms “reference” or “reference value” used interchangeably herein can refer to a measurement or characterization of a symptom after administration of the therapy (e.g., ketotifen, or a pharmaceutically acceptable salt thereof as described herein and/or a checkpoint inhibitor as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are likely to be alive after a particular duration of time.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a solid tumor by administration of ketotifen and/or a checkpoint inhibitor as described herein, refers to an amount of such ketotifen and/or checkpoint inhibitor that is sufficient to inhibit the occurrence or ameliorate one or more symptoms of a solid tumor. An effective amount of an antibody is administered in an “effective regimen.” The term “effective regimen” refers to a combination of amount of the ketotifen and/or checkpoint inhibitor being administered and dosage frequency adequate to accomplish prophylactic or therapeutic treatment of the disorder (e.g., prophylactic or therapeutic treatment of a solid tumor).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which ketotifen or a checkpoint inhibitor is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

The terms “inhibit” or “inhibition of” means to reduce by a measurable amount, or to prevent entirely. The term inhibition as used herein can refer to an inhibition or reduction of at least about 10%, at least about 15%, at least about 20%, at least about 25%, at least about 30%, at least about 35%, at least about 40%, at least about 50%, at least about 60%, at least about 70%, at least about 80%, at least about 90%, at least about 95%, or at least about 99%.

The terms “treatment” or “treat” refer to slowing, stopping, or reversing the progression of the disease or condition in a patient, as evidenced by a decrease or elimination of a clinical or diagnostic symptom of the disease or condition. Treatment can include, for example, a decrease in the severity of a symptom, the number of symptoms, or frequency of relapse.

The term “prodrug”, as used herein, refers to a compound that is converted into the active form of the compound upon administration in vivo. For example, a prodrug form of an active compound can be, but not limited to, acylated (acetylated or other) and ether derivatives, carboxylic esters or phosphate esters and various salt forms of the active compound. One of ordinary skill in the art will recognize how to readily modify the compound of subject invention to a prodrug form to facilitate delivery of active compound to a targeted site within the host organism or patient. The skilled artisan also will take advantage of favorable pharmacokinetic parameters of the prodrug form, where applicable, in delivering the desired compound to a targeted site within the host organism or patient to maximize the intended effect of the compound in the treatment of cancer.

As used herein, the term “synergy” or “synergistic effect” when used in connection with a description of the efficacy of a combination of agents, means any measured effect of the combination which is greater than the effect predicted from a sum of the effects of the individual agents.

As used herein, the term “additive” or “additive effect” when used in connection with a description of the efficacy of a combination of agents, means any measured effect of the combination which is similar to the effect predicted from a sum of the effects of the individual agents.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Ketotifen

The compound ketotifen is a relatively selective, non-competitive histamine antagonist (H1-receptor) and mast cell stabilizer. Ketotifen is known to inhibit the release of mediators in hypersensitivity reactions. Ketotifen has been administered both orally and in an ophthalmic form. Ketotifen has the following formula:

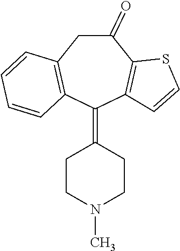 [SMILES: CN1CCC(=C2c3ccccc3CC(=O)c3sccc32)CC1]

In some embodiments, ketotifen as used herein refers to a salt, such as a salt with fumaric acid, ketotifen fumarate, having the formula:

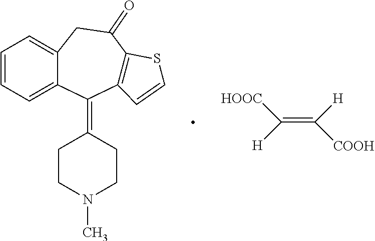 [SMILES: CN1CCC(=C2c3ccccc3CC(=O)c3sccc32)CC1 ; O=C(O)/C=C/C(=O)O]

In some embodiments of any of the described methods, the ketotifen is provided as a pharmaceutically acceptable salt of ketotifen. Preparations of ketotifen are disclosed, for example, in WO 2010/107525 A1, WO 2009/136903 A1, US 2010/160293 A1, and WO 2006/047418 A1.

### III. Checkpoint Inhibitors

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264) Therapy with antagonistic checkpoint blocking antibodies against immune system checkpoints such as CTLA4, PD1 and PD-L1 are one of the most promising new avenues of immunotherapy for cancer and other diseases. Additional checkpoint targets, such as TIM-3, LAG-3, various B-7 ligands, CHK 1 and CHK2 kinases, BTLA, A2aR, and others, are also under investigation. Checkpoint inhibitors include atezolizumab (Tecentriq®), a PD-L1 inhibitor, ipilimumab (Yervoy®), a CTLA-4 inhibitor, and pembrolizumab (Keytruda®) and nivolumab (Opdivo®), both PD-1 inhibitors.

Recent data suggest a secondary mechanism of anti-CTLA-4 antibodies, which could occur within the tumor itself. CTLA-4 has been found to be expressed in tumors at higher levels on regulatory T-cells (also referred to herein as “Treg cells”) as compared with intra-tumoral effector T-cells (also referred to herein as “Teff cells”), resulting in the hypothesis of anti-CTLA-4 preferentially impacting the Treg cell.

One mechanism by which the checkpoint blockade anti-CTLA-4 antibodies mediate anti-tumor effect is by decreasing regulatory T-cells. Due to the distinct mechanism of action of anti-CTLA-4 antibodies, they can successfully combine with the anti-PD-1 checkpoint blockade antibodies which work to release the suppressive signaling conferred to effector T-cells. Dual blockade with these antibodies combine to improve anti-tumor response both preclinically (Proc Natl Acad Sci USA 2010, 107, 4275-4280) and in the clinic (N Engl J Med 2013, 369, 122-133; N Engl J Med 2015, 372, 2006-2017).

In some embodiments, the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof. In some embodiments, the checkpoint inhibitor inhibits the checkpoint protein CTLA-4. In some embodiments, the checkpoint inhibitor inhibits the checkpoint protein PD-1. In some embodiments, the checkpoint inhibitor inhibits the checkpoint protein PD-L1. In some embodiments, the checkpoint inhibitor inhibits the checkpoint protein PD-L2. In some embodiments, the checkpoint inhibitor inhibits the checkpoint protein B7-H3. In some embodiments, the checkpoint inhibitor inhibits the checkpoint protein B7-H4. In some embodiments, the checkpoint inhibitor inhibits the checkpoint protein BMA. In some embodiments, the checkpoint inhibitor inhibits the checkpoint protein HVEM. In some embodiments, the checkpoint inhibitor inhibits the checkpoint protein TIM3. In some embodiments, the checkpoint inhibitor inhibits the checkpoint protein GAL9. In some embodiments, the checkpoint inhibitor inhibits the checkpoint protein LAG3. In some embodiments, the checkpoint inhibitor inhibits the checkpoint protein VISTA. In some embodiments, the checkpoint inhibitor inhibits the checkpoint protein KIR. In some embodiments, the checkpoint inhibitor inhibits the checkpoint protein 2B4. In some embodiments, the checkpoint inhibitor inhibits the checkpoint protein CD160. In some embodiments, the checkpoint inhibitor inhibits the checkpoint protein CGEN-15049. In some embodiments, the checkpoint inhibitor inhibits the checkpoint protein CHK1. In some embodiments, the checkpoint inhibitor inhibits the checkpoint protein CHK2. In some embodiments, the checkpoint inhibitor inhibits the checkpoint protein A2aR. In some embodiments, the checkpoint inhibitor inhibits B-7 family ligands. In some embodiments, the checkpoint is an antibody. In some embodiments, the checkpoint inhibitor is an anti-CTLA4 antibody. In some embodiments, the checkpoint inhibitor is an anti-PD-1 antibody. In some embodiments, the checkpoint inhibitor is an anti-PD-L1 antibody. In some embodiments, the checkpoint inhibitor is an anti-PD-L2 antibody. In some embodiments, the checkpoint inhibitor is an anti-B7-H3 antibody. In some embodiments, the checkpoint inhibitor is an anti-B7-H4 antibody. In some embodiments, the checkpoint inhibitor is an anti-BMA antibody. In some embodiments, the checkpoint inhibitor is an anti-HVEM antibody. In some embodiments, the checkpoint inhibitor is an anti-TIM3 antibody. In some embodiments, the checkpoint inhibitor is an anti-GAL9 antibody. In some embodiments, the checkpoint inhibitor is an anti-LAG3 antibody. In some embodiments, the checkpoint inhibitor is an anti-VISTA antibody. In some embodiments, the checkpoint inhibitor is an anti-KIR antibody. In some embodiments, the checkpoint inhibitor is an anti-2B4 antibody. In some embodiments, the checkpoint inhibitor is an anti-CD160 antibody. In some embodiments, the checkpoint inhibitor is an anti-CGEN-15049 antibody. In some embodiments, the checkpoint inhibitor is an anti-CHK1 antibody. In some embodiments, the checkpoint inhibitor is an anti-CHK2 antibody. In some embodiments, the checkpoint inhibitor is an anti-A2aR antibody. In some embodiments, the checkpoint inhibitor is an anti-B7 family ligand antibody. In some embodiments, the checkpoint inhibitor described herein is a monoclonal antibody. In some embodiments, the checkpoint inhibitor described herein is a human antibody. In some embodiments, the checkpoint inhibitor described herein is a humanized antibody. In some embodiments, the checkpoint inhibitor described herein is a chimeric antibody. In some embodiments, the checkpoint inhibitor described herein is a full-length antibody. In some embodiments, the checkpoint inhibitor described herein is an antigen-binding fragment of an antibody. In some embodiments, the antigen-binding fragment is selected from the group consisting of Fab, Fab′ and F(ab′)₂, Fd, single-chain Fvs (scFv), single-chain antibodies, disulfide-linked Fvs (sdFv) and fragments comprising either a V_{L} or V_{H} domain. In some embodiments, the checkpoint inhibitor described herein is antibody comprising the complementarity-determining regions (CDRs) of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the CDRs are the Kabat CDRs. Kabat et al. (1991), “Sequences of Proteins of Immunological Interest,” 5th Ed. Public Health Service, National Institutes of Health, Bethesda, MD (“Kabat” numbering scheme). In some embodiments, the checkpoint inhibitor described herein comprises the heavy chain variable region and/or the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the heavy chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein comprises the heavy chain variable region and the light chain variable region of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is a biosimilar of an antibody selected from the group consisting of MEDI0680, AMP-224, nivolumab, pembrolizumab, pidilizumab, MEDI4736, atezolizumab, ipilimumab, tremelimumab, and BMS-936559. In some embodiments, the checkpoint inhibitor described herein is MEDI0680. In some embodiments, the checkpoint inhibitor described herein is AMP-224. In some embodiments, the checkpoint inhibitor described herein is nivolumab. In some embodiments, the checkpoint inhibitor described herein is pembrolizumab. In some embodiments, the checkpoint inhibitor described herein is pidilizumab. In some embodiments, the checkpoint inhibitor described herein is MEDI4736. In some embodiments, the checkpoint inhibitor described herein is atezolizumab. In some embodiments, the checkpoint inhibitor described herein is ipilimumab. In some embodiments, the checkpoint inhibitor described herein is tremelimumab. In some embodiments, the checkpoint inhibitor described herein is BMS-936559. In some embodiments, the checkpoint inhibitor is a combination of an anti-PD-1 antibody and an anti-CTLA4 antibody. In some embodiments, the checkpoint inhibitor is a combination of nivolumab and ipilimumab. In some embodiments, the checkpoint inhibitor is a combination of pembrolizumab and ipilimumab. In some embodiments, the checkpoint inhibitor is a combination of an anti-PD-L1 antibody and an anti-CTLA4 antibody. In some embodiments, the checkpoint inhibitor is a combination of atezolizumab and ipilimumab.

### IV. Methods

### A. Treatment of Solid Tumors

In one aspect the invention provides a method for treating a solid tumor in a subject in need thereof comprising administering to the subject an effective amount of ketotifen, or a pharmaceutically acceptable salt thereof, in combination with a checkpoint inhibitor. In another aspect, the invention provides a method for initiating, enhancing or prolonging the effects of a checkpoint inhibitor, or enabling a subject to respond to a checkpoint inhibitor in a subject in need thereof comprising administering to the subject an effective amount of ketotifen, or a pharmaceutically acceptable salt thereof, in combination with a checkpoint inhibitor, wherein the subject has a solid tumor. In another aspect, the invention provides a method for potentiating the effects of a checkpoint inhibitor in a subject in need thereof comprising administering to the subject an effective amount of ketotifen, or a pharmaceutically acceptable salt thereof, in combination with a checkpoint inhibitor, wherein the subject has a solid tumor. In another aspect, the invention provides a method of increasing blood flow of a solid tumor in a subject comprising administering to the subject an effective amount of ketotifen, or a pharmaceutically acceptable salt thereof, in combination with a checkpoint inhibitor, wherein increasing blood flow of the solid tumor enhances the effect of the checkpoint inhibitor. In some embodiments, the blood flow of the solid tumor is determined using ultrasound-based blood flow measurements or using histological techniques to measure hypoxia. In some embodiments, the blood flow of the solid tumor is determined using ultrasound-based blood flow measurements. In some embodiments, the blood flow of the solid tumor is determined using histological techniques to measure hypoxia. In some embodiments, blood flow is measured using histological techniques to measure hypoxia in a biopsy from the solid tumor. In another aspect, the invention provides a method of improving the delivery or efficacy of a checkpoint inhibitor in a subject comprising administering an effective amount of ketotifen, or a pharmaceutically acceptable salt thereof, in combination with the checkpoint inhibitor, wherein the subject has a solid tumor, thereby improving the delivery or efficacy of the therapy in the subject. In some embodiments, the subject is a human. In some embodiments, the method further comprises administering an effective amount of ketotifen, or a pharmaceutically acceptable salt thereof, in combination with an effective amount of an additional chemotherapeutic agent, such as doxorubicin. In some embodiments, the method further comprises administering an effective amount of ketotifen, or a pharmaceutically acceptable salt thereof, in combination with an effective amount of an additional chemotherapeutic agent, such as doxorubicin, and in combination with a checkpoint inhibitor. In some embodiments, the additional chemotherapeutic agent is doxorubicin.

Doxorubicin is a small compound having the formula of CAS 23214-92-8 and sold under the trade name adriamycin. DOXIL® is the trade name of a polyethylene glycol-coated liposome encapsulated form of doxorubicin.

In another aspect, the invention provides a method of determining an effective amount of ketotifen in a subject with a solid tumor comprising: (a) measuring the blood flow and/or stiffness of the solid tumor; (b) administering to the subject an effective amount of ketotifen; and (c) measuring the blood flow and/or stiffness of the solid tumor after administering ketotifen, wherein an increase in blood flow and/or a decrease in stiffness following administration of ketotifen to the subject indicates that the amount administered was an effective amount. In another aspect, the invention provides a method for treating a solid tumor in a subject in need thereof comprising: (a) measuring the blood flow and/or stiffness of the solid tumor; (b) administering to the subject an effective amount of ketotifen; (c) measuring the blood flow and/or stiffness of the solid tumor after administering the ketotifen; and (d) administering a chemotherapeutic agent if the blood flow of the solid tumor is increased and/or the stiffness of the solid tumor is decreased after administering the ketotifen. In another aspect, the invention provides a method for treating a solid tumor in a subject in need thereof comprising: (a) measuring the blood flow and/or stiffness of the solid tumor; (b) administering to the subject an effective amount of ketotifen; (c) measuring the blood flow and/or stiffness of the solid tumor after administering the ketotifen; (d) determining that the subject is responsive to a chemotherapeutic agent based on an increase in the blood flow of the solid tumor or a decrease in the stiffness of the solid tumor after administering the ketotifen; and (e) administering the chemotherapeutic agent to the subject who has been determined to be responsive to the chemotherapeutic agent based on the increase in the blood flow of the solid tumor or the decrease in the stiffness of the solid tumor after administering the ketotifen. In another aspect, the invention provides a method for predicting response to treatment with a chemotherapeutic agent comprising: (a) measuring the blood flow and/or stiffness of the solid tumor; (b) administering to the subject an effective amount of ketotifen; (c) measuring the blood flow and/or stiffness of the solid tumor after administering the ketotifen, wherein an increase in the blood flow of the solid tumor or a decrease in the stiffness of the solid tumor after administering the ketotifen indicates that the subject is likely to respond to treatment with the chemotherapeutic agent. In some embodiments, the effective amount of ketotifen is determined by measuring the change in blood flow and/or stiffness of the solid tumor following administration of the ketotifen to the subject, wherein an increase in blood flow and/or a decrease in stiffness following administration of the ketotifen to the subject indicates that the amount administered was an effective amount. In some embodiments, the method comprises measuring the blood flow of the solid tumor and the blood flow of the solid tumor is increased after administering the ketotifen. In some embodiments, the method comprises measuring the stiffness of the solid tumor and the stiffness of the solid tumor is decreased after administering the ketotifen. In some embodiments, the ketotifen is administered for at least 1 day, at least 2 days, at least 3 days, at least 4 days, or at least 5 days prior to the administration of the chemotherapeutic agent. In some embodiments, the ketotifen is administered at a dose that increases the blood flow of the solid tumor and/or decreases the stiffness of the solid tumor. In some embodiments, blood flow and/or stiffness of the solid tumor is measured using ultrasound. In some embodiments, blood flow of the solid tumor is measured using histological techniques to measure hypoxia. In some embodiments, the chemotherapeutic agent is a checkpoint inhibitor. In some embodiments, the subject is a human.

In some embodiments of any of the aspects provided herein, administering ketotifen reduces the tissue stiffness of the solid tumor. In some embodiments of any of the aspects provided herein, administering ketotifen, or pharmaceutically acceptable salt thereof, reduces the tissue stiffness of the solid tumor. In some embodiments, tissue stiffness of the solid tumor is reduced by at least 10%. In some embodiments, tissue stiffness of the solid tumor is reduced by at least 20%. In some embodiments, tissue stiffness of the solid tumor is reduced by at least 25%. In some embodiments, tissue stiffness of the solid tumor is reduced by at least 30%. In some embodiments, tissue stiffness of the solid tumor is reduced by at least 40%. In some embodiments, tissue stiffness of the solid tumor is reduced by at least 50%. In some embodiments, tissue stiffness of the solid tumor is reduced by at least 60%. In some embodiments, tissue stiffness of the solid tumor is reduced by at least 70%. In some embodiments, tissue stiffness of the solid tumor is reduced by at least 75%. In some embodiments, tissue stiffness of the solid tumor is measured using ultrasound elastography.

In some embodiments of any of the aspects provided herein, administering ketotifen decreases the levels of an extracellular matrix protein in the solid tumor. In some embodiments of any of the aspects provided herein, administering ketotifen, or pharmaceutically acceptable salt thereof, decreases the levels of an extracellular matrix protein in the solid tumor. In some embodiments, the levels of an extracellular matrix protein in the solid tumor are reduced by at least 10%. In some embodiments, the levels of an extracellular matrix protein in the solid tumor are reduced by at least 20%. In some embodiments, the levels of an extracellular matrix protein in the solid tumor are reduced by at least 25%. In some embodiments, the levels of an extracellular matrix protein in the solid tumor are reduced by at least 30%. In some embodiments, the levels of an extracellular matrix protein in the solid tumor are reduced by at least 40%. In some embodiments, the levels of an extracellular matrix protein in the solid tumor are reduced by at least 50%. In some embodiments, the levels of an extracellular matrix protein in the solid tumor are reduced by at least 60%. In some embodiments, the levels of an extracellular matrix protein in the solid tumor are reduced by at least 70%. In some embodiments, the levels of an extracellular matrix protein in the solid tumor are reduced by at least 75%. In some embodiments, the extracellular matrix protein is collagen I. In some embodiments, the extracellular matrix protein is hyaluronan binding protein (HABP).

In some embodiments of any of the aspects provided herein, administering ketotifen reduces hypoxia in the solid tumor. In some embodiments of any of the aspects provided herein, administering ketotifen, or pharmaceutically acceptable salt thereof, reduces hypoxia in the solid tumor. In some embodiments, hypoxia is reduced by at least 10%. In some embodiments, hypoxia is reduced by at least 20%. In some embodiments, hypoxia is reduced by at least 25%. In some embodiments, hypoxia is reduced by at least 30%. In some embodiments, hypoxia is reduced by at least 40%. In some embodiments, hypoxia is reduced by at least 50%. In some embodiments, hypoxia is reduced by at least 60%. In some embodiments, hypoxia is reduced by at least 70%. In some embodiments, hypoxia is reduced by at least 75%.

In some embodiments of any of the aspects provided herein, the solid tumor is selected from the group consisting of mesothelioma, breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma. In some embodiments, the solid tumor is breast cancer. In some embodiments, the breast cancer has high tumor endothelin-B receptor expression relative to non-tumor tissue. In some embodiments, the breast cancer is triple negative breast cancer. In some embodiments, the solid tumor is mesothelioma. In some embodiments, the solid tumor is a lung metastasis from breast cancer. In some embodiments, the solid tumor is a sarcoma. In some embodiments, the sarcoma is osteosarcoma. In some embodiments, the sarcoma is fibrosarcoma. In some embodiments, the solid tumor is pancreatic cancer. In some embodiments, the solid tumor is ovarian cancer. In some embodiments, the solid tumor is a liver metastasis. In some embodiments, the liver metastasis is from colorectal cancer. In some embodiments, the solid tumor is a prostate cancer. In some embodiments, the prostate cancer has high tumor endothelin-A receptor expression relative to non-tumor tissue. In some embodiments, the solid cancer is a brain cancer. In some embodiments, the brain cancer has high tumor endothelin-A receptor expression relative to non-tumor tissue. In some embodiments, the solid tumor is melanoma. In some embodiments, the solid tumor is renal cell carcinoma. In some embodiments, the solid tumor is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the solid tumor is hepatocellular carcinoma. In some embodiments, the solid tumor is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor is squamous cell carcinoma of the head and neck. In some embodiments, the solid tumor is urothelial carcinoma. In some embodiments, the solid tumor is esophageal squamous cell carcinoma. In some embodiments, the solid tumor is gastric cancer. In some embodiments, the solid tumor is esophageal cancer. In some embodiments, the solid tumor is cervical cancer. In some embodiments, the solid tumor is Merkel cell carcinoma. In some embodiments, the solid tumor is endometrial carcinoma. In some embodiments, the solid tumor is cutaneous squamous cell carcinoma. In some embodiments, the solid tumor is a cancer that has compressed blood vessels and/or is hypoperfused. In some embodiments, the solid tumor is a cancer that has compressed blood vessels. In some embodiments, the solid tumor is a cancer that is hypoperfused. In some embodiments, the solid tumor that has compressed blood vessels and/or is hypoperfused is selected from the group consisting of breast cancer, breast cancer lung metastases, pancreatic cancer, ovarian cancer, and liver metastases. In some embodiments, the solid tumor that has compressed blood vessels and/or is hypoperfused is breast cancer. In some embodiments, the breast cancer has high tumor endothelin-B receptor expression relative to non-tumor tissue. In some embodiments, the breast cancer is triple negative breast cancer. In some embodiments, the solid tumor that has compressed blood vessels and/or is hypoperfused is pancreatic cancer. In some embodiments, the solid tumor that has compressed blood vessels and/or is hypoperfused is ovarian cancer. In some embodiments, the solid tumor that has compressed blood vessels and/or is hypoperfused is a liver metastasis. In some embodiments, the liver metastasis is from colorectal cancer. In some embodiments, the solid tumor that has compressed blood vessels and/or is hypoperfused is a lung metastasis. In some embodiments, the liver metastasis is from breast cancer. In some embodiments, the solid tumor is a cancer that has endothelin receptor expression in the tumor vasculature and/or fibroblasts. In some embodiments, the solid tumor is a cancer that has endothelin receptor expression in the tumor vasculature. In some embodiments, the solid tumor is a cancer that has endothelin receptor expression in the tumor fibroblasts. In some embodiments, the solid tumor that has endothelin receptor expression in the tumor vasculature and/or fibroblasts is selected from the group consisting of pancreatic cancer, ovarian cancer, lung cancer, prostate cancer, brain cancer, breast cancer, and colorectal cancer. In some embodiments, the solid tumor that has endothelin receptor expression in the tumor vasculature and/or fibroblasts is pancreatic cancer. In some embodiments, the solid tumor that has endothelin receptor expression in the tumor vasculature and/or fibroblasts is ovarian cancer. In some embodiments, the solid tumor that has endothelin receptor expression in the tumor vasculature and/or fibroblasts is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor that has endothelin receptor expression in the tumor vasculature and/or fibroblasts is prostate cancer. In some embodiments, the prostate cancer has high tumor endothelin-A receptor expression relative to non-tumor tissue. In some embodiments, the solid tumor that has endothelin receptor expression in the tumor vasculature and/or fibroblasts is brain cancer. In some embodiments, the brain cancer has high tumor endothelin-A receptor expression relative to non-tumor tissue. In some embodiments, the solid tumor that has endothelin receptor expression in the tumor vasculature and/or fibroblasts is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor that has endothelin receptor expression in the tumor vasculature and/or fibroblasts is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue.

### B. Routes of Administration

Chemotherapeutic agents described herein can be administered by any suitable route and mode. Ketotifen, or a pharmaceutically acceptable salt thereof, or a checkpoint inhibitor described herein can be administered by any suitable route and mode. Suitable routes of administering compounds or antibodies of the present invention are well known in the art and may be selected by those of ordinary skill in the art. In one embodiment, the ketotifen, or pharmaceutically acceptable salt thereof, and/or the checkpoint inhibitor described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of the chemotherapeutic agent is intraperitoneal injection. In some embodiments, the route of administration of the chemotherapeutic agent is intravenous injection. In some embodiments, the route of administration of ketotifen, or pharmaceutically acceptable salt thereof, is intraperitoneal injection. In some embodiments, the route of administration of the checkpoint inhibitor is intraperitoneal injection. In some embodiments, the route of administration of ketotifen, or pharmaceutically acceptable salt thereof, is intravenous injection. In some embodiments, the route of administration of the checkpoint inhibitor is intravenous injection. In one embodiment, the ketotifen, or pharmaceutically acceptable salt thereof, and/or the checkpoint inhibitor described herein are administered enterally. In some embodiments, the route of administration of ketotifen, or pharmaceutically acceptable salt thereof, is enteral. In some embodiments, the route of administration of ketotifen, or pharmaceutically acceptable salt thereof, is oral. In some embodiments, the route of administration of the checkpoint inhibitor is enteral. In some embodiments, the route of administration of the checkpoint inhibitor is oral. In some embodiments, the route of administration of the chemotherapeutic agent is enteral. In some embodiments, the route of administration of the chemotherapeutic agent is oral.

### C. Dosage and Frequency of Administration

In one aspect, the present invention provides for methods as described herein comprising administering ketotifen, or a pharmaceutically acceptable salt thereof, as described herein and a checkpoint inhibitor as described herein, wherein the subject is administered the ketotifen, or a pharmaceutically acceptable salt thereof, as described herein and the checkpoint inhibitor as described herein with particular frequencies. In another aspect, the present invention provides for methods as described herein comprising administering an ketotifen as described herein and a chemotherapeutic agent as described herein, wherein the subject is administered the ketotifen as described herein and the chemotherapeutic agent as described herein with particular frequencies.

In one embodiment of the methods or uses or product for uses provided herein an ketotifen as described herein is administered to the subject in a therapeutically effective amount. In one embodiment of the methods or uses or product for uses provided herein ketotifen, or a pharmaceutically acceptable salt thereof, as described herein is administered to the subject in a therapeutically effective amount. In one embodiment of the methods or uses or product for uses provided herein ketotifen, or a pharmaceutically acceptable salt thereof, as described herein is administered to the subject at a subtherapeutic dose. In one embodiment of the methods or uses or product for uses provided herein ketotifen, or a pharmaceutically acceptable salt thereof, as described herein is administered to the subject at a dose that is sufficient to initiate the effects of a checkpoint inhibitor. In one embodiment of the methods or uses or product for uses provided herein ketotifen, or a pharmaceutically acceptable salt thereof, as described herein is administered to the subject at a dose that is sufficient to enhance the effects of a checkpoint inhibitor. In one embodiment of the methods or uses or product for uses provided herein ketotifen, or a pharmaceutically acceptable salt thereof, as described herein is administered to the subject at a dose that is sufficient to prolong the effects of a checkpoint inhibitor. In one embodiment of the methods or uses or product for uses provided herein ketotifen, or a pharmaceutically acceptable salt thereof, as described herein is administered to the subject at a dose that is sufficient to potentiate the effects of a checkpoint inhibitor. In one embodiment of the methods or uses or product for uses provided herein ketotifen, or a pharmaceutically acceptable salt thereof, as described herein is administered to the subject at a dose that is sufficient to improve the delivery of a checkpoint inhibitor to a solid tumor. In one embodiment of the methods or uses or product for uses provided herein ketotifen, or a pharmaceutically acceptable salt thereof, as described herein is administered to the subject at a dose that is sufficient to improve the efficacy of a checkpoint inhibitor. In one embodiment of the methods or uses or product for uses provided herein ketotifen, or a pharmaceutically acceptable salt thereof, as described herein is administered to the subject at a dose that is sufficient to increase the number of anti-tumor T cells that colocalize with a solid tumor. In one embodiment of the methods or uses or product for uses provided herein ketotifen, or a pharmaceutically acceptable salt thereof, as described herein is administered to the subject at a dose that is sufficient to reduce the tissue stiffness of a solid tumor. In one embodiment of the methods or uses or product for uses provided herein ketotifen, or a pharmaceutically acceptable salt thereof, as described herein is administered to the subject at a dose that is sufficient to decrease the levels of an extracellular matrix protein in a solid tumor. In one embodiment of the methods or uses or product for uses provided herein ketotifen, or a pharmaceutically acceptable salt thereof, as described herein is administered to the subject at a dose that is sufficient to increase blood flow of a solid tumor. In one embodiment of the methods or uses or product for uses provided herein ketotifen, or a pharmaceutically acceptable salt thereof, as described herein is administered to the subject at a dose that is sufficient decrease the levels of an extracellular matrix protein in a solid tumor and increase blood flow of the solid tumor. In one embodiment of the methods or uses or product for uses provided herein ketotifen, or a pharmaceutically acceptable salt thereof, as described herein is administered to the subject at a dose that is sufficient to reduces hypoxia in a solid tumor.

In some embodiments of the methods or uses or product for uses provided herein an ketotifen as described herein is administered to the subject at a dose ranging from about 0.01 mg/kg to about 20 mg/kg of the subject's body weight. In some embodiments of the methods or uses or product for uses provided herein ketotifen, or a pharmaceutically acceptable salt thereof, as described herein is administered to the subject at a dose ranging from about 0.01 mg/kg to about 20 mg/kg of the subject's body weight. In one embodiment, ketotifen, or a pharmaceutically acceptable salt thereof, as described herein is administered at a dose ranging from about 0.05 mg/kg to about 15 mg/kg of the subject's body weight. In one embodiment, ketotifen, or a pharmaceutically acceptable salt thereof, as described herein is administered at a dose ranging from about 0.01 mg/kg to about 0.1 mg/kg of the subject's body weight. In one embodiment, ketotifen, or a pharmaceutically acceptable salt thereof, as described herein is administered at a dose ranging from about 0.01 mg/kg to about 0.5 mg/kg of the subject's body weight. In one embodiment, ketotifen, or a pharmaceutically acceptable salt thereof, as described herein is administered at a dose ranging from about 0.01 mg/kg to about 1.0 mg/kg of the subject's body weight. In one embodiment, ketotifen, or a pharmaceutically acceptable salt thereof, as described herein is administered at a dose ranging from about 0.01 mg/kg to about 5 mg/kg of the subject's body weight. In one embodiment, ketotifen, or a pharmaceutically acceptable salt thereof, as described herein is administered at a dose ranging from about 0.05 mg/kg to about 0.1 mg/kg of the subject's body weight. In one embodiment, ketotifen, or a pharmaceutically acceptable salt thereof, as described herein is administered at a dose ranging from about 0.05 mg/kg to about 10 mg/kg of the subject's body weight. In one embodiment, ketotifen, or a pharmaceutically acceptable salt thereof, as described herein is administered at a dose ranging from about 0.05 mg/kg to about 5 mg/kg of the subject's body weight. In one embodiment, ketotifen, or a pharmaceutically acceptable salt thereof, as described herein is administered at a dose ranging from about 0.05 mg/kg to about 3 mg/kg of the subject's body weight. In one embodiment, ketotifen, or a pharmaceutically acceptable salt thereof, as described herein is administered at a dose ranging from about 0.25 mg/kg to about 10 mg/kg of the subject's body weight. In one embodiment, ketotifen, or a pharmaceutically acceptable salt thereof, as described herein is administered at a dose ranging from about 0.25 mg/kg to about 5 mg/kg of the subject's body weight. In one embodiment, ketotifen, or a pharmaceutically acceptable salt thereof, as described herein is administered at a dose ranging from about 0.25 mg/kg to about 3 mg/kg of the subject's body weight. In one embodiment, ketotifen, or a pharmaceutically acceptable salt thereof, as described herein is administered at a dose ranging from about 0.5 mg/kg to about 10 mg/kg of the subject's body weight. In one embodiment, ketotifen, or a pharmaceutically acceptable salt thereof, as described herein is administered at a dose ranging from about 0.5 mg/kg to about 5 mg/kg of the subject's body weight. In one embodiment, ketotifen, or a pharmaceutically acceptable salt thereof, as described herein is administered at a dose ranging from about 0.5 mg/kg to about 3 mg/kg of the subject's body weight. In one embodiment, ketotifen, or a pharmaceutically acceptable salt thereof, as described herein is administered at a dose ranging from about 0.75 mg/kg to about 10 mg/kg of the subject's body weight. In one embodiment, ketotifen, or a pharmaceutically acceptable salt thereof, as described herein is administered at a dose ranging from about 0.75 mg/kg to about 5 mg/kg of the subject's body weight. In one embodiment, ketotifen, or a pharmaceutically acceptable salt thereof, as described herein is administered at a dose ranging from about 0.75 mg/kg to about 3 mg/kg of the subject's body weight. In one embodiment, ketotifen, or a pharmaceutically acceptable salt thereof, as described herein is administered at a dose ranging from about 1 mg/kg to about 10 mg/kg of the subject's body weight. In one embodiment, ketotifen, or a pharmaceutically acceptable salt thereof, as described herein is administered at a dose ranging from about 1 mg/kg to about 5.0 mg/kg of the subject's body weight. In one embodiment, ketotifen, or a pharmaceutically acceptable salt thereof, as described herein is administered at a dose ranging from about 1 mg/kg to about 3 mg/kg of the subject's body weight. In one embodiment, ketotifen, or a pharmaceutically acceptable salt thereof, as described herein is administered at a dose ranging from about 2 mg/kg to about 20 mg/kg of the subject's body weight. In one embodiment, ketotifen, or a pharmaceutically acceptable salt thereof, as described herein is administered at a dose ranging from about 2 mg/kg to about 15 mg/kg of the subject's body weight. In one embodiment, ketotifen, or a pharmaceutically acceptable salt thereof, as described herein is administered at a dose ranging from about 2 mg/kg to about 10 mg/kg of the subject's body weight. In one embodiment, ketotifen, or a pharmaceutically acceptable salt thereof, as described herein is administered at a dose ranging from about 2 mg/kg to about 5 mg/kg of the subject's body weight. In one embodiment, ketotifen, or a pharmaceutically acceptable salt thereof, as described herein is administered at a dose ranging from about 4 mg/kg to about 20 mg/kg of the subject's body weight. In one embodiment, ketotifen, or a pharmaceutically acceptable salt thereof, as described herein is administered at a dose ranging from about 4 mg/kg to about 15 mg/kg of the subject's body weight. In one embodiment, ketotifen, or a pharmaceutically acceptable salt thereof, as described herein is administered at a dose ranging from about 4 mg/kg to about 10 mg/kg of the subject's body weight. In one embodiment, ketotifen, or a pharmaceutically acceptable salt thereof, as described herein is administered at a dose ranging from about 4 mg/kg to about 5 mg/kg of the subject's body weight. In one embodiment, ketotifen, or a pharmaceutically acceptable salt thereof, as described herein is administered at a dose ranging from about 5 mg/kg to about 20 mg/kg of the subject's body weight. In one embodiment, ketotifen, or a pharmaceutically acceptable salt thereof, as described herein is administered at a dose of about 0.01 mg/kg of the subject's body weight. In one embodiment, ketotifen, or a pharmaceutically acceptable salt thereof, as described herein is administered at a dose of about 0.05 mg/kg of the subject's body weight. In one embodiment, ketotifen, or a pharmaceutically acceptable salt thereof, as described herein is administered at a dose of about 0.1 mg/kg of the subject's body weight. In one embodiment, ketotifen, or a pharmaceutically acceptable salt thereof, as described herein is administered at a dose of about 0.15 mg/kg of the subject's body weight. In one embodiment, ketotifen, or a pharmaceutically acceptable salt thereof, as described herein is administered at a dose of about 0.16 mg/kg of the subject's body weight. In one embodiment, ketotifen, or a pharmaceutically acceptable salt thereof, as described herein is administered at a dose of about 0.2 mg/kg of the subject's body weight. In one embodiment, ketotifen, or a pharmaceutically acceptable salt thereof, as described herein is administered at a dose of about 0.3 mg/kg of the subject's body weight. In one embodiment, ketotifen, or a pharmaceutically acceptable salt thereof, as described herein is administered at a dose of about 0.4 mg/kg of the subject's body weight. In one embodiment, ketotifen, or a pharmaceutically acceptable salt thereof, as described herein is administered at a dose of about 0.5 mg/kg of the subject's body weight. In one embodiment, ketotifen, or a pharmaceutically acceptable salt thereof, as described herein is administered at a dose of about 0.6 mg/kg of the subject's body weight. In one embodiment, ketotifen, or a pharmaceutically acceptable salt thereof, as described herein is administered at a dose of about 0.7 mg/kg of the subject's body weight. In one embodiment, ketotifen, or a pharmaceutically acceptable salt thereof, as described herein is administered at a dose of about 0.8 mg/kg of the subject's body weight. In one embodiment, ketotifen, or a pharmaceutically acceptable salt thereof, as described herein is administered at a dose of about 0.9 mg/kg of the subject's body weight. In one embodiment, ketotifen, or a pharmaceutically acceptable salt thereof, as described herein is administered at a dose of about 1 mg/kg of the subject's body weight. In one embodiment, ketotifen, or a pharmaceutically acceptable salt thereof, as described herein is administered at a dose of about 1.2 mg/kg of the subject's body weight. In one embodiment, ketotifen, or a pharmaceutically acceptable salt thereof, as described herein is administered at a dose of about 1.4 mg/kg of the subject's body weight. In one embodiment, ketotifen, or a pharmaceutically acceptable salt thereof, as described herein is administered at a dose of about 1.6 mg/kg of the subject's body weight. In one embodiment, ketotifen, or a pharmaceutically acceptable salt thereof, as described herein is administered at a dose of about 1.8 mg/kg of the subject's body weight. In one embodiment, ketotifen, or a pharmaceutically acceptable salt thereof, as described herein is administered at a dose of about 2 mg/kg of the subject's body weight. In one embodiment, ketotifen, or a pharmaceutically acceptable salt thereof, as described herein is administered at a dose of about 2.2 mg/kg of the subject's body weight. In one embodiment, ketotifen, or a pharmaceutically acceptable salt thereof, as described herein is administered at a dose of about 2.4 mg/kg of the subject's body weight. In one embodiment, ketotifen, or a pharmaceutically acceptable salt thereof, as described herein is administered at a dose of about 2.6 mg/kg of the subject's body weight. In one embodiment, ketotifen, or a pharmaceutically acceptable salt thereof, as described herein is administered at a dose of about 2.8 mg/kg of the subject's body weight. In one embodiment, ketotifen, or a pharmaceutically acceptable salt thereof, as described herein is administered at a dose of about 3 mg/kg of the subject's body weight. In one embodiment, ketotifen, or a pharmaceutically acceptable salt thereof, as described herein is administered at a dose of about 3.2 mg/kg of the subject's body weight. In one embodiment, ketotifen, or a pharmaceutically acceptable salt thereof, as described herein is administered at a dose of about 3.4 mg/kg of the subject's body weight. In one embodiment, ketotifen, or a pharmaceutically acceptable salt thereof, as described herein is administered at a dose of about 3.6 mg/kg of the subject's body weight. In one embodiment, ketotifen, or a pharmaceutically acceptable salt thereof, as described herein is administered at a dose of about 3.8 mg/kg of the subject's body weight. In one embodiment, ketotifen, or a pharmaceutically acceptable salt thereof, as described herein is administered at a dose of about 4 mg/kg of the subject's body weight. In one embodiment, ketotifen, or a pharmaceutically acceptable salt thereof, as described herein is administered at a dose of about 4.2 mg/kg of the subject's body weight. In one embodiment, ketotifen, or a pharmaceutically acceptable salt thereof, as described herein is administered at a dose of about 4.4 mg/kg of the subject's body weight. In one embodiment, ketotifen, or a pharmaceutically acceptable salt thereof, as described herein is administered at a dose of about 4.6 mg/kg of the subject's body weight. In one embodiment, ketotifen, or a pharmaceutically acceptable salt thereof, as described herein is administered at a dose of about 4.8 mg/kg of the subject's body weight. In one embodiment, ketotifen, or a pharmaceutically acceptable salt thereof, as described herein is administered at a dose of about 5 mg/kg of the subject's body weight. In one embodiment, ketotifen, or a pharmaceutically acceptable salt thereof, as described herein is administered at a dose of about 5.2 mg/kg of the subject's body weight. In one embodiment, ketotifen, or a pharmaceutically acceptable salt thereof, as described herein is administered at a dose of about 5.4 mg/kg of the subject's body weight. In one embodiment, ketotifen, or a pharmaceutically acceptable salt thereof, as described herein is administered at a dose of about 5.6 mg/kg of the subject's body weight. In one embodiment, ketotifen, or a pharmaceutically acceptable salt thereof, as described herein is administered at a dose of about 5.8 mg/kg of the subject's body weight. In one embodiment, ketotifen, or a pharmaceutically acceptable salt thereof, as described herein is administered at a dose of about 6 mg/kg of the subject's body weight. In one embodiment, ketotifen, or a pharmaceutically acceptable salt thereof, as described herein is administered at a dose of about 6.5 mg/kg of the subject's body weight. In one embodiment, ketotifen, or a pharmaceutically acceptable salt thereof, as described herein is administered at a dose of about 7 mg/kg of the subject's body weight. In one embodiment, ketotifen, or a pharmaceutically acceptable salt thereof, as described herein is administered at a dose of about 7.5 mg/kg of the subject's body weight. In one embodiment, ketotifen, or a pharmaceutically acceptable salt thereof, as described herein is administered at a dose of about 8 mg/kg of the subject's body weight. In one embodiment, ketotifen, or a pharmaceutically acceptable salt thereof, as described herein is administered at a dose of about 8.5 mg/kg of the subject's body weight. In one embodiment, ketotifen, or a pharmaceutically acceptable salt thereof, as described herein is administered at a dose of about 9 mg/kg of the subject's body weight. In one embodiment, ketotifen, or a pharmaceutically acceptable salt thereof, as described herein is administered at a dose of about 9.5 mg/kg of the subject's body weight. In one embodiment, ketotifen, or a pharmaceutically acceptable salt thereof, as described herein is administered at a dose of about 10 mg/kg of the subject's body weight. In one embodiment, ketotifen, or a pharmaceutically acceptable salt thereof, as described herein is administered at a dose of about 11 mg/kg of the subject's body weight. In one embodiment, ketotifen, or a pharmaceutically acceptable salt thereof, as described herein is administered at a dose of about 12 mg/kg of the subject's body weight. In one embodiment, ketotifen, or a pharmaceutically acceptable salt thereof, as described herein is administered at a dose of about 13 mg/kg of the subject's body weight. In one embodiment, ketotifen, or a pharmaceutically acceptable salt thereof, as described herein is administered at a dose of about 14 mg/kg of the subject's body weight. In one embodiment, ketotifen, or a pharmaceutically acceptable salt thereof, as described herein is administered at a dose of about 15 mg/kg of the subject's body weight. In one embodiment, ketotifen, or a pharmaceutically acceptable salt thereof, as described herein is administered at a dose of about 16 mg/kg of the subject's body weight. In one embodiment, ketotifen, or a pharmaceutically acceptable salt thereof, as described herein is administered at a dose of about 17 mg/kg of the subject's body weight. In one embodiment, ketotifen, or a pharmaceutically acceptable salt thereof, as described herein is administered at a dose of about 18 mg/kg of the subject's body weight. In one embodiment, ketotifen, or a pharmaceutically acceptable salt thereof, as described herein is administered at a dose of about 19 mg/kg of the subject's body weight. In one embodiment, ketotifen, or a pharmaceutically acceptable salt thereof, as described herein is administered at a dose of about 20 mg/kg of the subject's body weight.

In some embodiments of the methods or uses or product for uses provided herein the ketotifen as described herein is administered to the subject at a dose ranging from about 10 mg to about 1250 mg. In one embodiment, ketotifen, or a pharmaceutically acceptable salt thereof, as described herein is administered at a dose ranging from about 10 mg to about 150 mg. In one embodiment, ketotifen, or a pharmaceutically acceptable salt thereof, as described herein is administered at a dose ranging from about 10 mg to about 100 mg. In one embodiment, ketotifen, or a pharmaceutically acceptable salt thereof, as described herein is administered at a dose ranging from about 10 mg to about 50 mg. In one embodiment, ketotifen, or a pharmaceutically acceptable salt thereof, as described herein is administered at a dose ranging from about 25 mg to about 150 mg. In one embodiment, ketotifen, or a pharmaceutically acceptable salt thereof, as described herein is administered at a dose ranging from about 25 mg to about 100 mg. In one embodiment, ketotifen, or a pharmaceutically acceptable salt thereof, as described herein is administered at a dose ranging from about 25 mg to about 50 mg. In one embodiment, ketotifen, or a pharmaceutically acceptable salt thereof, as described herein is administered at a dose ranging from about 50 mg to about 150 mg. In one embodiment, ketotifen, or a pharmaceutically acceptable salt thereof, as described herein is administered at a dose ranging from about 50 mg to about 100 mg. In one embodiment, ketotifen, or a pharmaceutically acceptable salt thereof, as described herein is administered at a dose ranging from about 50 mg to about 75 mg. In one embodiment, ketotifen, or a pharmaceutically acceptable salt thereof, as described herein is administered at a dose ranging from about 75 mg to about 150 mg. In one embodiment, ketotifen, or a pharmaceutically acceptable salt thereof, as described herein is administered at a dose ranging from about 75 mg to about 100 mg. In one embodiment, ketotifen, or a pharmaceutically acceptable salt thereof, as described herein is administered at a dose ranging from about 100 mg to about 1200 mg. In one embodiment, ketotifen, or a pharmaceutically acceptable salt thereof, as described herein is administered at a dose ranging from about 10 mg to about 40 mg. In one embodiment, ketotifen, or a pharmaceutically acceptable salt thereof, as described herein is administered at a dose ranging from about 10 mg to about 30 mg. In one embodiment, ketotifen, or a pharmaceutically acceptable salt thereof, as described herein is administered at a dose ranging from about 10 mg to about 20 mg. In one embodiment, ketotifen, or a pharmaceutically acceptable salt thereof, as described herein is administered at a dose ranging from about 15 mg to about 40 mg. In one embodiment, ketotifen, or a pharmaceutically acceptable salt thereof, as described herein is administered at a dose ranging from about 20 mg to about 40 mg. In one embodiment, ketotifen, or a pharmaceutically acceptable salt thereof, as described herein is administered at a dose ranging from about 30 mg to about 40 mg. In one embodiment, ketotifen, or a pharmaceutically acceptable salt thereof, as described herein is administered at a dose of about 10 mg. In one embodiment, ketotifen, or a pharmaceutically acceptable salt thereof, as described herein is administered at a dose of about 15 mg. In one embodiment, ketotifen, or a pharmaceutically acceptable salt thereof, as described herein is administered at a dose of about 20 mg. In one embodiment, ketotifen, or a pharmaceutically acceptable salt thereof, as described herein is administered at a dose of about 25 mg. In one embodiment, ketotifen, or a pharmaceutically acceptable salt thereof, as described herein is administered at a dose of about 30 mg. In one embodiment, ketotifen, or a pharmaceutically acceptable salt thereof, as described herein is administered at a dose of about 35 mg. In one embodiment, ketotifen, or a pharmaceutically acceptable salt thereof, as described herein is administered at a dose of about 40 mg. In one embodiment, ketotifen, or a pharmaceutically acceptable salt thereof, as described herein is administered at a dose of about 45 mg. In one embodiment, ketotifen, or a pharmaceutically acceptable salt thereof, as described herein is administered at a dose of about 50 mg. In one embodiment, ketotifen, or a pharmaceutically acceptable salt thereof, as described herein is administered at a dose of about 55 mg. In one embodiment, ketotifen, or a pharmaceutically acceptable salt thereof, as described herein is administered at a dose of about 60 mg. In one embodiment, ketotifen, or a pharmaceutically acceptable salt thereof, as described herein is administered at a dose of about 62.5 mg. In one embodiment, ketotifen, or a pharmaceutically acceptable salt thereof, as described herein is administered at a dose of about 65 mg. In one embodiment, ketotifen, or a pharmaceutically acceptable salt thereof, as described herein is administered at a dose of about 70 mg. In one embodiment, ketotifen, or a pharmaceutically acceptable salt thereof, as described herein is administered at a dose of about 75 mg. In one embodiment, ketotifen, or a pharmaceutically acceptable salt thereof, as described herein is administered at a dose of about 80 mg. In one embodiment, ketotifen, or a pharmaceutically acceptable salt thereof, as described herein is administered at a dose of about 85 mg. In one embodiment, ketotifen, or a pharmaceutically acceptable salt thereof, as described herein is administered at a dose of about 90 mg. In one embodiment, ketotifen, or a pharmaceutically acceptable salt thereof, as described herein is administered at a dose of about 95 mg. In one embodiment, ketotifen, or a pharmaceutically acceptable salt thereof, as described herein is administered at a dose of about 100 mg. In one embodiment, ketotifen, or a pharmaceutically acceptable salt thereof, as described herein is administered at a dose of about 105 mg. In one embodiment, ketotifen, or a pharmaceutically acceptable salt thereof, as described herein is administered at a dose of about 110 mg. In one embodiment, ketotifen, or a pharmaceutically acceptable salt thereof, as described herein is administered at a dose of about 115 mg. In one embodiment, ketotifen, or a pharmaceutically acceptable salt thereof, as described herein is administered at a dose of about 120 mg. In one embodiment, ketotifen, or a pharmaceutically acceptable salt thereof, as described herein is administered at a dose of about 125 mg. In one embodiment, ketotifen, or a pharmaceutically acceptable salt thereof, as described herein is administered at a dose of about 130 mg. In one embodiment, ketotifen, or a pharmaceutically acceptable salt thereof, as described herein is administered at a dose of about 135 mg. In one embodiment, ketotifen, or a pharmaceutically acceptable salt thereof, as described herein is administered at a dose of about 140 mg. In one embodiment, ketotifen, or a pharmaceutically acceptable salt thereof, as described herein is administered at a dose of about 145 mg. In one embodiment, ketotifen, or a pharmaceutically acceptable salt thereof, as described herein is administered at a dose of about 150 mg. In one embodiment, ketotifen, or a pharmaceutically acceptable salt thereof, as described herein is administered at a dose of about 175 mg. In one embodiment, ketotifen, or a pharmaceutically acceptable salt thereof, as described herein is administered at a dose of about 200 mg. In one embodiment, ketotifen, or a pharmaceutically acceptable salt thereof, as described herein is administered at a dose of about 250 mg. In one embodiment, ketotifen, or a pharmaceutically acceptable salt thereof, as described herein is administered at a dose of about 300 mg. In one embodiment, ketotifen, or a pharmaceutically acceptable salt thereof, as described herein is administered at a dose of about 350 mg. In one embodiment, ketotifen, or a pharmaceutically acceptable salt thereof, as described herein is administered at a dose of about 400 mg. In one embodiment, ketotifen, or a pharmaceutically acceptable salt thereof, as described herein is administered at a dose of about 450 mg. In one embodiment, ketotifen, or a pharmaceutically acceptable salt thereof, as described herein is administered at a dose of about 500 mg. In one embodiment, ketotifen, or a pharmaceutically acceptable salt thereof, as described herein is administered at a dose of about 550 mg. In one embodiment, ketotifen, or a pharmaceutically acceptable salt thereof, as described herein is administered at a dose of about 600 mg. In one embodiment, ketotifen, or a pharmaceutically acceptable salt thereof, as described herein is administered at a dose of about 650 mg. In one embodiment, ketotifen, or a pharmaceutically acceptable salt thereof, as described herein is administered at a dose of about 700 mg. In one embodiment, ketotifen, or a pharmaceutically acceptable salt thereof, as described herein is administered at a dose of about 750 mg. In one embodiment, ketotifen, or a pharmaceutically acceptable salt thereof, as described herein is administered at a dose of about 800 mg. In one embodiment, ketotifen, or a pharmaceutically acceptable salt thereof, as described herein is administered at a dose of about 850 mg. In one embodiment, ketotifen, or a pharmaceutically acceptable salt thereof, as described herein is administered at a dose of about 900 mg. In one embodiment, ketotifen, or a pharmaceutically acceptable salt thereof, as described herein is administered at a dose of about 950 mg. In one embodiment, ketotifen, or a pharmaceutically acceptable salt thereof, as described herein is administered at a dose of about 1000 mg. In one embodiment, ketotifen, or a pharmaceutically acceptable salt thereof, as described herein is administered at a dose of about 1050 mg. In one embodiment, ketotifen, or a pharmaceutically acceptable salt thereof, as described herein is administered at a dose of about 1100 mg. In one embodiment, ketotifen, or a pharmaceutically acceptable salt thereof, as described herein is administered at a dose of about 1150 mg. In one embodiment, ketotifen, or a pharmaceutically acceptable salt thereof, as described herein is administered at a dose of about 1200 mg. In one embodiment, ketotifen, or a pharmaceutically acceptable salt thereof, as described herein is administered at a dose of about 1250 mg.

In one embodiment of the methods or uses or product for uses provided herein, ketotifen is administered to the subject daily, twice daily, three times daily or four times daily. In some embodiments, ketotifen, or a pharmaceutically acceptable salt thereof, is administered to the subject every other day, once about every week or once about every three weeks. In some embodiments, ketotifen, or a pharmaceutically acceptable salt thereof, is administered to the subject about once per day. In some embodiments, ketotifen, or a pharmaceutically acceptable salt thereof, is administered to the subject about twice per day. In some embodiments, ketotifen, or a pharmaceutically acceptable salt thereof, is administered to the subject once per day. In some embodiments, ketotifen, or a pharmaceutically acceptable salt thereof, is administered to the subject twice per day. In some embodiments, ketotifen, or a pharmaceutically acceptable salt thereof, is administered to the subject orally.

In some embodiments of the methods or uses or product for uses provided herein a chemotherapeutic agent as described herein is administered to the subject at a dose ranging from about 0.5 mg/kg to about 15 mg/kg of the subject's body weight. In some embodiments of the methods or uses or product for uses provided herein a checkpoint inhibitor as described herein is administered to the subject at a dose ranging from about 0.5 mg/kg to about 15 mg/kg of the subject's body weight. In one embodiment, a checkpoint inhibitor as described herein is administered at a dose ranging from about 1 mg/kg to about 10 mg/kg. In one embodiment, a checkpoint inhibitor as described herein is administered at a dose of about 1 mg/kg of the subject's body weight. In one embodiment, a checkpoint inhibitor as described herein is administered at a dose of about 2 mg/kg of the subject's body weight. In one embodiment, a checkpoint inhibitor as described herein is administered at a dose of about 3 mg/kg of the subject's body weight. In one embodiment, a checkpoint inhibitor as described herein is administered at a dose of about 4 mg/kg of the subject's body weight. In one embodiment, a checkpoint inhibitor as described herein is administered at a dose of about 5 mg/kg of the subject's body weight. In one embodiment, a checkpoint inhibitor as described herein is administered at a dose of about 6 mg/kg of the subject's body weight. In one embodiment, a checkpoint inhibitor as described herein is administered at a dose of about 7 mg/kg of the subject's body weight. In one embodiment, a checkpoint inhibitor as described herein is administered at a dose of about 8 mg/kg of the subject's body weight. In one embodiment, a checkpoint inhibitor as described herein is administered at a dose of about 9 mg/kg of the subject's body weight. In one embodiment, a checkpoint inhibitor as described herein is administered at a dose of about 10 mg/kg of the subject's body weight. In one embodiment, a checkpoint inhibitor as described herein is administered at a dose of about 11 mg/kg of the subject's body weight. In one embodiment, a checkpoint inhibitor as described herein is administered at a dose of about 12 mg/kg of the subject's body weight. In one embodiment, a checkpoint inhibitor as described herein is administered at a dose of about 13 mg/kg of the subject's body weight. In one embodiment, a checkpoint inhibitor as described herein is administered at a dose of about 14 mg/kg of the subject's body weight. In one embodiment, a checkpoint inhibitor as described herein is administered at a dose of about 15 mg/kg of the subject's body weight. In one embodiment, a checkpoint inhibitor as described herein is administered at a dose of about 2 mg/kg and the checkpoint inhibitor is pembrolizumab. In one embodiment, a checkpoint inhibitor as described herein is administered at a dose of about 1 mg/kg and the checkpoint inhibitor is nivolumab. In one embodiment, a checkpoint inhibitor as described herein is administered at a dose of about 3 mg/kg and the checkpoint inhibitor is nivolumab. In one embodiment, a checkpoint inhibitor as described herein is administered at a dose of about 1 mg/kg and the checkpoint inhibitor is ipilimumab. In one embodiment, a checkpoint inhibitor as described herein is administered at a dose of about 3 mg/kg and the checkpoint inhibitor is ipilimumab. In one embodiment, a checkpoint inhibitor as described herein is administered at a dose of about 10 mg/kg and the checkpoint inhibitor is ipilimumab.

In some embodiments of the methods or uses or product for uses provided herein a chemotherapeutic agent as described herein is administered to the subject at a dose ranging from about 100 mg to about 2000 mg. In some embodiments of the methods or uses or product for uses provided herein a checkpoint inhibitor as described herein is administered to the subject at a dose ranging from about 100 mg to about 2000 mg. In one embodiment, a checkpoint inhibitor as described herein is administered at a dose ranging from about 200 mg to about 1800 mg. In one embodiment, a checkpoint inhibitor as described herein is administered at a dose ranging from about 200 mg to about 400 mg. In one embodiment, a checkpoint inhibitor as described herein is administered at a dose ranging from about 400 mg to about 600 mg. In one embodiment, a checkpoint inhibitor as described herein is administered at a dose ranging from about 600 mg to about 1000 mg. In one embodiment, a checkpoint inhibitor as described herein is administered at a dose ranging from about 800 mg to about 1000 mg. In one embodiment, a checkpoint inhibitor as described herein is administered at a dose ranging from about 1000 mg to about 1800 mg. In one embodiment, a checkpoint inhibitor as described herein is administered at a dose ranging from about 1000 mg to about 1600 mg. In one embodiment, a checkpoint inhibitor as described herein is administered at a dose ranging from about 1000 mg to about 1300 mg. In one embodiment, a checkpoint inhibitor as described herein is administered at a dose ranging from about 140 mg to about 1800 mg. In one embodiment, a checkpoint inhibitor as described herein is administered at a dose ranging from about 1600 mg to about 1800 mg. In one embodiment, a checkpoint inhibitor as described herein is administered at a dose of about 100 mg. In one embodiment, a checkpoint inhibitor as described herein is administered at a dose of about 200 mg. In one embodiment, a checkpoint inhibitor as described herein is administered at a dose of about 240 mg. In one embodiment, a checkpoint inhibitor as described herein is administered at a dose of about 300 mg. In one embodiment, a checkpoint inhibitor as described herein is administered at a dose of about 360 mg. In one embodiment, a checkpoint inhibitor as described herein is administered at a dose of about 400 mg. In one embodiment, a checkpoint inhibitor as described herein is administered at a dose of about 480 mg. In one embodiment, a checkpoint inhibitor as described herein is administered at a dose of about 500 mg. In one embodiment, a checkpoint inhibitor as described herein is administered at a dose of about 600 mg. In one embodiment, a checkpoint inhibitor as described herein is administered at a dose of about 700 mg. In one embodiment, a checkpoint inhibitor as described herein is administered at a dose of about 800 mg. In one embodiment, a checkpoint inhibitor as described herein is administered at a dose of about 840 mg. In one embodiment, a checkpoint inhibitor as described herein is administered at a dose of about 900 mg. In one embodiment, a checkpoint inhibitor as described herein is administered at a dose of about 1000 mg. In one embodiment, a checkpoint inhibitor as described herein is administered at a dose of about 1100 mg. In one embodiment, a checkpoint inhibitor as described herein is administered at a dose of about 1200 mg. In one embodiment, a checkpoint inhibitor as described herein is administered at a dose of about 1300 mg. In one embodiment, a checkpoint inhibitor as described herein is administered at a dose of about 1400 mg. In one embodiment, a checkpoint inhibitor as described herein is administered at a dose of about 1500 mg. In one embodiment, a checkpoint inhibitor as described herein is administered at a dose of about 1600 mg. In one embodiment, a checkpoint inhibitor as described herein is administered at a dose of about 1700 mg. In one embodiment, a checkpoint inhibitor as described herein is administered at a dose of about 1800 mg. In one embodiment, a checkpoint inhibitor as described herein is administered at a dose of about 1900 mg. In one embodiment, a checkpoint inhibitor as described herein is administered at a dose of about 2000 mg. In one embodiment, a checkpoint inhibitor as described herein is administered at a dose of about 200 mg and the checkpoint inhibitor is pembrolizumab. In one embodiment, a checkpoint inhibitor as described herein is administered at a dose of about 400 mg and the checkpoint inhibitor is pembrolizumab. In one embodiment, a checkpoint inhibitor as described herein is administered at a dose of about 240 mg and the checkpoint inhibitor is nivolumab. In one embodiment, a checkpoint inhibitor as described herein is administered at a dose of about 480 mg and the checkpoint inhibitor is nivolumab. In one embodiment, a checkpoint inhibitor as described herein is administered at a dose of about 360 mg and the checkpoint inhibitor is nivolumab. In one embodiment, a checkpoint inhibitor as described herein is administered at a dose of about 840 mg and the checkpoint inhibitor is atezolizumab. In one embodiment, a checkpoint inhibitor as described herein is administered at a dose of about 1200 mg and the checkpoint inhibitor is atezolizumab. In one embodiment, a checkpoint inhibitor as described herein is administered at a dose of about 1680 mg and the checkpoint inhibitor is atezolizumab.

In one embodiment of the methods or uses or product for uses provided herein, a chemotherapeutic agent as described herein is administered to the subject daily, twice daily, three times daily or four times daily. In one embodiment of the methods or uses or product for uses provided herein, a checkpoint inhibitor as described herein is administered to the subject daily, twice daily, three times daily or four times daily. In some embodiments, a checkpoint inhibitor as described herein is administered once about every week to once about every 8 weeks. In some embodiments, a checkpoint inhibitor described herein is administered once about 1 every week. In some embodiments, a checkpoint inhibitor described herein is administered once about 2 every weeks. In some embodiments, a checkpoint inhibitor described herein is administered once about 3 every weeks. In some embodiments, a checkpoint inhibitor described herein is administered once about 4 every weeks. In some embodiments, a checkpoint inhibitor described herein is administered once about 5 every weeks. In some embodiments, a checkpoint inhibitor described herein is administered once about 6 every weeks. In some embodiments, a checkpoint inhibitor described herein is administered once about 7 every weeks. In some embodiments, a checkpoint inhibitor described herein is administered once about 8 every weeks. In some embodiments, a checkpoint inhibitor described herein is administered once about every 3 weeks and the checkpoint inhibitor is pembrolizumab. In some embodiments, a checkpoint inhibitor described herein is administered at a dose of about 200 mg once about every 3 weeks and the checkpoint inhibitor is pembrolizumab. In some embodiments, a checkpoint inhibitor described herein is administered once about every 6 weeks and the checkpoint inhibitor is pembrolizumab. In some embodiments, a checkpoint inhibitor described herein is administered at a dose of about 400 mg once about every 6 weeks and the checkpoint inhibitor is pembrolizumab. In some embodiments, a checkpoint inhibitor described herein is administered at a dose of about 2 mg/kg of the subject's body weight once about every 3 weeks and the checkpoint inhibitor is pembrolizumab. In some embodiments, a checkpoint inhibitor described herein is administered once about every 2 weeks and the checkpoint inhibitor is nivolumab. In some embodiments, a checkpoint inhibitor described herein is administered at a dose of about 240 mg once about every 2 weeks and the checkpoint inhibitor is nivolumab. In some embodiments, a checkpoint inhibitor described herein is administered once about every 3 weeks and the checkpoint inhibitor is nivolumab. In some embodiments, a checkpoint inhibitor described herein is administered at a dose of about 360 mg once about every 3 weeks and the checkpoint inhibitor is nivolumab. In some embodiments, a checkpoint inhibitor described herein is administered once about every 4 weeks and the checkpoint inhibitor is nivolumab. In some embodiments, a checkpoint inhibitor described herein is administered at a dose of about 480 mg once about every 4 weeks and the checkpoint inhibitor is nivolumab. In some embodiments, a checkpoint inhibitor described herein is administered at a dose of about 1 mg/kg once about every 3 weeks and the checkpoint inhibitor is nivolumab. In some embodiments, a checkpoint inhibitor described herein is administered at a dose of about 3 mg/kg once about every 2 weeks and the checkpoint inhibitor is nivolumab. In some embodiments, a checkpoint inhibitor described herein is administered at a dose of about 3 mg/kg once about every 3 weeks and the checkpoint inhibitor is nivolumab. In some embodiments, a checkpoint inhibitor described herein is administered once about every 3 weeks and the checkpoint inhibitor is ipilimumab. In some embodiments, a checkpoint inhibitor described herein is administered once about every 6 weeks and the checkpoint inhibitor is ipilimumab. In some embodiments, a checkpoint inhibitor described herein is administered at a dose of about 1 mg/kg once about every 3 weeks and the checkpoint inhibitor is ipilimumab. In some embodiments, a checkpoint inhibitor described herein is administered at a dose of about 3 mg/kg once about every 3 weeks and the checkpoint inhibitor is ipilimumab. In some embodiments, a checkpoint inhibitor described herein is administered at a dose of about 10 mg/kg once about every 3 weeks and the checkpoint inhibitor is ipilimumab. In some embodiments, a checkpoint inhibitor described herein is administered at a dose of about 10 mg/kg once about every 12 weeks and the checkpoint inhibitor is ipilimumab. In some embodiments, a checkpoint inhibitor described herein is administered at a dose of about 1 mg/kg once about every 6 weeks and the checkpoint inhibitor is ipilimumab. In some embodiments, a checkpoint inhibitor described herein is administered once about every 2 weeks and the checkpoint inhibitor is atezolizumab. In some embodiments, a checkpoint inhibitor described herein is administered at a dose of about 840 mg once about every 2 weeks and the checkpoint inhibitor is atezolizumab. In some embodiments, a checkpoint inhibitor described herein is administered once about every 3 weeks and the checkpoint inhibitor is atezolizumab. In some embodiments, a checkpoint inhibitor described herein is administered at a dose of about 1200 mg once about every 3 weeks and the checkpoint inhibitor is atezolizumab. In some embodiments, a checkpoint inhibitor described herein is administered once about every 4 weeks and the checkpoint inhibitor is atezolizumab. In some embodiments, a checkpoint inhibitor described herein is administered at a dose of about 1680 mg once about every 4 weeks and the checkpoint inhibitor is atezolizumab. In some embodiments, a checkpoint inhibitor as described herein is administered to the subject by intravenous infusion.

### D. Treatment Outcome

In one aspect, a method of treating cancer with an ketotifen as described herein and a chemotherapeutic agent as described herein results in an improvement in one or more therapeutic effects in the subject after administration relative to a baseline. In one aspect, a method of treating cancer with ketotifen, or a pharmaceutically acceptable salt thereof, as described herein and a checkpoint inhibitor as described herein results in an improvement in one or more therapeutic effects in the subject after administration relative to a baseline. In some embodiments, the one or more therapeutic effects is the size of the tumor derived from the cancer (e.g., solid tumor), the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an ketotifen as described herein and a chemotherapeutic agent as described herein may include the RECIST Criteria 1.1. In one embodiment of the methods or uses or product for uses provided herein, response to treatment with ketotifen, or pharmaceutically acceptable salt thereof, as described herein and a checkpoint inhibitor as described herein may include the RECIST Criteria 1.1. The RECIST Criteria 1.1 are as follows:

| | Category | Criteria |
|---|---|---|
| Based on | Complete | Disappearance of all target lesions. Any |
| target lesions | Response (CR) | pathological lymph nodes must have reduction in |
| | | short axis to <10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and >5 mm) increase in the sum of the LDs |
| | Disease (PD) | of target lesions, taking as reference the smallest |
| | | sum of the target LDs recorded while in trial or the |
| | | appearance of one or more new lesions. |
| Based on non- | CR | Disappearance of all non-target lesions and |
| target lesions | | normalization of tumor marker level. All lymph |
| | | nodes must be non-pathological in size (<10 mm |
| | | short axis). |
| | SD | Persistence of one or more non-target lesion(s) |
| | | or/and maintenance of tumor marker level above the |
| | | normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with an ketotifen as described herein and a chemotherapeutic agent as described herein is assessed by measuring the objective response rate. In one embodiment of the methods or uses or product for uses provided herein, the effectiveness of treatment with ketotifen, or pharmaceutically acceptable salt thereof, as described herein and a checkpoint inhibitor as described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an ketotifen as described herein and a chemotherapeutic agent as described herein is assessed by measuring the size of a tumor derived from the cancer (e.g., solid tumor). In one embodiment of the methods or uses or product for uses provided herein, response to treatment with ketotifen, or a pharmaceutically acceptable salt thereof, as described herein and a checkpoint inhibitor as described herein is assessed by measuring the size of a tumor derived from the cancer (e.g., solid tumor). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of ketotifen, or a pharmaceutically acceptable salt thereof, as described herein and/or a checkpoint inhibitor as described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of ketotifen, or a pharmaceutically acceptable salt thereof, as described herein and/or a checkpoint inhibitor as described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of the tumor derived from the cancer is reduced relative to the size of the tumor before administration of ketotifen, or a pharmaceutically acceptable salt thereof, as described herein and a checkpoint inhibitor as described herein. In some embodiments, the size of the tumor derived from the cancer is reduced relative to the size of the tumor before administration of ketotifen, or a pharmaceutically acceptable salt thereof, as described herein. In some embodiments, the size of the tumor derived from the cancer is reduced relative to the size of the tumor before administration of a checkpoint inhibitor as described herein.

In one embodiment of the methods or uses or product for uses described herein, response to treatment with ketotifen as described herein and a chemotherapeutic agent as described herein is assessed by measuring the time of progression free survival after administration of the ketotifen as described herein and/or the chemotherapeutic agent as described herein. In one embodiment of the methods or uses or product for uses described herein, response to treatment with ketotifen, or a pharmaceutically acceptable salt thereof, as described herein and a checkpoint inhibitor as described herein is assessed by measuring the time of progression free survival after administration of ketotifen, or a pharmaceutically acceptable salt thereof, as described herein and/or a checkpoint inhibitor as described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of ketotifen, or a pharmaceutically acceptable salt thereof, as described herein and/or a checkpoint inhibitor as described herein. In some embodiments, the subject exhibits progression-free survival of at least about 6 months after administration of ketotifen, or a pharmaceutically acceptable salt thereof, as described herein and/or a checkpoint inhibitor as described herein. In some embodiments, the subject exhibits progression-free survival of at least about one year after administration of ketotifen, or a pharmaceutically acceptable salt thereof, as described herein and/or a checkpoint inhibitor as described herein. In some embodiments, the subject exhibits progression-free survival of at least about two years after administration of ketotifen, or a pharmaceutically acceptable salt thereof, as described herein and/or a checkpoint inhibitor as described herein. In some embodiments, the subject exhibits progression-free survival of at least about three years after administration of ketotifen, or a pharmaceutically acceptable salt thereof, as described herein and/or a checkpoint inhibitor as described herein. In some embodiments, the subject exhibits progression-free survival of at least about four years after administration of ketotifen, or a pharmaceutically acceptable salt thereof, as described herein and/or a checkpoint inhibitor as described herein. In some embodiments, the subject exhibits progression-free survival of at least about five years after administration of ketotifen, or a pharmaceutically acceptable salt thereof, as described herein and/or a checkpoint inhibitor as described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of ketotifen, or a pharmaceutically acceptable salt thereof, as described herein and/or a checkpoint inhibitor as described herein. In some embodiments, the subject exhibits progression-free survival of at least 6 months after administration of ketotifen, or a pharmaceutically acceptable salt thereof, as described herein and/or a checkpoint inhibitor as described herein. In some embodiments, the subject exhibits progression-free survival of at least one year after administration of ketotifen, or a pharmaceutically acceptable salt thereof, as described herein and/or a checkpoint inhibitor as described herein. In some embodiments, the subject exhibits progression-free survival of at least two years after administration of ketotifen, or a pharmaceutically acceptable salt thereof, as described herein and/or a checkpoint inhibitor as described herein. In some embodiments, the subject exhibits progression-free survival of at least three years after administration of ketotifen, or a pharmaceutically acceptable salt thereof, as described herein and/or a checkpoint inhibitor as described herein. In some embodiments, the subject exhibits progression-free survival of at least four years after administration of ketotifen, or a pharmaceutically acceptable salt thereof, as described herein and/or a checkpoint inhibitor as described herein. In some embodiments, the subject exhibits progression-free survival of at least five years after administration of ketotifen, or a pharmaceutically acceptable salt thereof, as described herein and/or a checkpoint inhibitor as described herein. In some embodiments, response to treatment is assessed by measuring the time of progression free survival after administration of ketotifen, or a pharmaceutically acceptable salt thereof, as described herein and a checkpoint inhibitor as described herein. In some embodiments, response to treatment is assessed by measuring the time of progression free survival after administration of ketotifen, or a pharmaceutically acceptable salt thereof, as described herein. In some embodiments, response to treatment is assessed by measuring the time of progression free survival after administration of a checkpoint inhibitor as described herein.

In one embodiment of the methods or uses or product for uses described herein, response to treatment with an ketotifen as described herein and a chemotherapeutic agent as described herein is assessed by measuring the time of overall survival after administration of the ketotifen as described herein and/or the chemotherapeutic agent as described herein. In one embodiment of the methods or uses or product for uses described herein, response to treatment with ketotifen, or a pharmaceutically acceptable salt thereof, as described herein and a checkpoint inhibitor as described herein is assessed by measuring the time of overall survival after administration of ketotifen, or a pharmaceutically acceptable salt thereof, as described herein and/or a checkpoint inhibitor as described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of ketotifen, or a pharmaceutically acceptable salt thereof, as described herein and/or a checkpoint inhibitor as described herein. In some embodiments, the subject exhibits overall survival of at least about 6 months after administration of ketotifen, or a pharmaceutically acceptable salt thereof, as described herein and/or a checkpoint inhibitor as described herein. In some embodiments, the subject exhibits overall survival of at least about one year after administration of ketotifen, or a pharmaceutically acceptable salt thereof, as described herein and/or a checkpoint inhibitor as described herein. In some embodiments, the subject exhibits overall survival of at least about two years after administration of ketotifen, or a pharmaceutically acceptable salt thereof, as described herein and/or a checkpoint inhibitor as described herein. In some embodiments, the subject exhibits overall survival of at least about three years after administration of ketotifen, or a pharmaceutically acceptable salt thereof, as described herein and/or a checkpoint inhibitor as described herein. In some embodiments, the subject exhibits overall survival of at least about four years after administration of ketotifen, or a pharmaceutically acceptable salt thereof, as described herein and/or a checkpoint inhibitor as described herein. In some embodiments, the subject exhibits overall survival of at least about five years after administration of ketotifen, or a pharmaceutically acceptable salt thereof, as described herein and/or a checkpoint inhibitor as described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of ketotifen, or a pharmaceutically acceptable salt thereof, as described herein and/or a checkpoint inhibitor as described herein. In some embodiments, the subject exhibits overall survival of at least 6 months after administration of ketotifen, or a pharmaceutically acceptable salt thereof, as described herein and/or a checkpoint inhibitor as described herein. In some embodiments, the subject exhibits overall survival of at least one year after administration of ketotifen, or a pharmaceutically acceptable salt thereof, as described herein and/or a checkpoint inhibitor as described herein. In some embodiments, the subject exhibits overall survival of at least two years after administration of ketotifen, or a pharmaceutically acceptable salt thereof, as described herein and/or a checkpoint inhibitor as described herein. In some embodiments, the subject exhibits overall survival of at least three years after administration of ketotifen, or a pharmaceutically acceptable salt thereof, as described herein and/or a checkpoint inhibitor as described herein. In some embodiments, the subject exhibits overall survival of at least four years after administration of ketotifen, or a pharmaceutically acceptable salt thereof, as described herein and/or a checkpoint inhibitor as described herein. In some embodiments, the subject exhibits overall survival of at least five years after administration of ketotifen, or a pharmaceutically acceptable salt thereof, as described herein and/or a checkpoint inhibitor as described herein. In some embodiments, response to treatment is assessed by measuring the time of overall survival after administration of ketotifen, or a pharmaceutically acceptable salt thereof, as described herein and a checkpoint inhibitor as described herein. In some embodiments, response to treatment is assessed by measuring the time of overall survival after administration of ketotifen, or a pharmaceutically acceptable salt thereof, as described herein. In some embodiments, response to treatment is assessed by measuring the time of overall survival after administration of a checkpoint inhibitor as described herein.

In one embodiment of the methods or uses or product for uses described herein, response to treatment with an ketotifen as described herein and a chemotherapeutic agent as described herein is assessed by measuring the duration of response to the ketotifen as described herein and the chemotherapeutic agent as described herein after administration of the ketotifen as described herein and/or the chemotherapeutic agent as described herein. In one embodiment of the methods or uses or product for uses described herein, response to treatment with ketotifen, or a pharmaceutically acceptable salt thereof, as described herein and a checkpoint inhibitor as described herein is assessed by measuring the duration of response to ketotifen, or a pharmaceutically acceptable salt thereof, as described herein and a checkpoint inhibitor as described herein after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about 6 months after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about one year after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about two years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about three years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about four years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least about five years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least 6 months after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least one year after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least two years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least three years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least four years after administration of ketotifen, or a pharmaceutically acceptable salt thereof, as described herein and/or a checkpoint inhibitor as described herein. In some embodiments, the duration of response to ketotifen, or a pharmaceutically acceptable salt thereof, as described herein and a checkpoint inhibitor as described herein is at least five years after administration of ketotifen, or a pharmaceutically acceptable salt thereof, as described herein and/or a checkpoint inhibitor as described herein. In some embodiments, the duration of response is measured after administration of the ketotifen, or a pharmaceutically acceptable salt thereof, as described herein and a checkpoint inhibitor as described herein. In some embodiments, the duration of response is measured after administration of ketotifen, or a pharmaceutically acceptable salt thereof, as described herein. In some embodiments, the duration of response is measured after administration of a checkpoint inhibitor as described herein.

### V. Compositions

In some aspects, also provided herein are compositions (e.g., pharmaceutical compositions and therapeutic formulations) comprising ketotifen as described herein and/or a chemotherapeutic agent as described herein. In some aspects, also provided herein are compositions (e.g., pharmaceutical compositions and therapeutic formulations) comprising ketotifen, or a pharmaceutically acceptable salt thereof, as described herein and/or a checkpoint inhibitor as described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In some embodiments, a formulation comprising ketotifen comprises ketotifen fumarate. In some embodiments, a formulation comprising ketotifen comprises ketotifen fumarate dissolved in ddH₂O. In some embodiments, the ketotifen is prepared as a liquid formulation. In some embodiments, the ketotifen is prepared as an ophthalmic solution. In some embodiments, the ketotifen is prepared as a solid, such as a tablet.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

In some embodiments, a composition comprising ketotifen as described herein is coadministered with a composition comprising a chemotherapeutic agent as described herein. In some embodiments, a composition comprising ketotifen, or a pharmaceutically acceptable salt thereof, as described herein is coadministered with a composition comprising a checkpoint inhibitor as described herein. In some embodiments the coadministration is simultaneous or sequential. In some embodiments, ketotifen, or a pharmaceutically acceptable salt thereof, as described herein is administered simultaneously with a checkpoint inhibitor as described herein. In some embodiments, simultaneous means that ketotifen, or a pharmaceutically acceptable salt thereof, as described herein and the checkpoint inhibitor as described herein are administered to the subject less than about one hour apart, such as less than about 30 minutes apart, less than about 15 minutes apart, less than about 10 minutes apart or less than about 5 minutes apart. In some embodiments, simultaneous means that ketotifen, or a pharmaceutically acceptable salt thereof, as described herein and the checkpoint inhibitor as described herein are administered to the subject less than one hour apart, such as less than 30 minutes apart, less than 15 minutes apart, less than 10 minutes apart or less than 5 minutes apart. In some embodiments, ketotifen, or a pharmaceutically acceptable salt thereof, as described herein is administered sequentially with the checkpoint inhibitor as described herein. In some embodiments, sequential administration means that ketotifen, or a pharmaceutically acceptable salt thereof, as described herein and the checkpoint inhibitor as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, ketotifen, or a pharmaceutically acceptable salt thereof, as described herein is administered prior to the administration of the checkpoint inhibitor as described herein. In some embodiments, ketotifen, or a pharmaceutically acceptable salt thereof, as described herein is administered to the subject beginning at least 1 day prior to the subject being administered the checkpoint inhibitor as described herein. In some embodiments, ketotifen, or a pharmaceutically acceptable salt thereof, as described herein is administered to the subject beginning at least 2 days prior to the subject being administered the checkpoint inhibitor as described herein. In some embodiments, ketotifen, or a pharmaceutically acceptable salt thereof, as described herein is administered to the subject beginning at least 3 days prior to the subject being administered the checkpoint inhibitor as described herein. In some embodiments, ketotifen, or a pharmaceutically acceptable salt thereof, as described herein is administered to the subject beginning at least 4 days prior to the subject being administered the checkpoint inhibitor as described herein. In some embodiments, ketotifen, or a pharmaceutically acceptable salt thereof, as described herein is administered to the subject beginning at least 5 days prior to the subject being administered the checkpoint inhibitor as described herein. In some embodiments, ketotifen, or a pharmaceutically acceptable salt thereof, as described herein is administered to the subject beginning at least 1 week prior to the subject being administered the checkpoint inhibitor as described herein. In some embodiments, ketotifen, or a pharmaceutically acceptable salt thereof, as described herein is administered to the subject beginning at least 2 weeks prior to the subject being administered the checkpoint inhibitor as described herein. In some embodiments, ketotifen, or a pharmaceutically acceptable salt thereof, as described herein is administered to the subject beginning at least 3 weeks prior to the subject being administered the checkpoint inhibitor as described herein. In some embodiments, ketotifen, or a pharmaceutically acceptable salt thereof, as described herein is administered to the subject beginning at least 4 weeks prior to the subject being administered the checkpoint inhibitor as described herein. In some embodiments, ketotifen, or a pharmaceutically acceptable salt thereof, as described herein is administered to the subject beginning prior to the subject being administered the checkpoint inhibitor as described herein and administration is maintained for at least a portion of the time the subject is administered the checkpoint inhibitor. In some embodiments, ketotifen, or a pharmaceutically acceptable salt thereof, as described herein is administered to the subject beginning prior to the subject being administered the checkpoint inhibitor as described herein and administration is maintained for the entire period of time the subject is administered the checkpoint inhibitor.

In some embodiments, a composition comprising ketotifen, or a pharmaceutically acceptable salt thereof, as described herein and/or a checkpoint inhibitor as described herein is coadministered with one or additional therapeutic agents. In some embodiments the coadministration is simultaneous or sequential.

### VI. Articles of Manufacture and Kits

In another aspect, an article of manufacture or kit is provided which comprises an ketotifen as described herein and/or a chemotherapeutic agent as described herein. In another aspect, an article of manufacture or kit is provided which comprises ketotifen, or a pharmaceutically acceptable salt thereof, as described herein and/or a checkpoint inhibitor as described herein. The article of manufacture or kit may further comprise instructions for use of ketotifen, or a pharmaceutically acceptable salt thereof, as described herein and/or a checkpoint inhibitor as described herein in the methods of the invention. Thus, in certain embodiments, the article of manufacture or kit comprises instructions for the use of ketotifen, or a pharmaceutically acceptable salt thereof, as described herein and/or a checkpoint inhibitor as described herein in methods for treating cancer (e.g., solid tumors) in a subject comprising administering to the subject an effective amount of ketotifen, or a pharmaceutically acceptable salt thereof, as described herein and/or a checkpoint inhibitor as described herein. In some embodiments of any of the aspects provided herein, the solid tumor is selected from the group consisting of mesothelioma, breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma. In some embodiments, the solid tumor is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor is a sarcoma. In some embodiments, the sarcoma is osteosarcoma. In some embodiments, the sarcoma is fibrosarcoma. In some embodiments, the solid tumor is pancreatic cancer. In some embodiments, the solid tumor is ovarian cancer. In some embodiments, the solid tumor is a liver metastasis. In some embodiments, the liver metastasis is from colorectal cancer. In some embodiments, the solid tumor is a prostate cancer. In some embodiments, the prostate cancer has high tumor endothelin-A receptor expression relative to non-tumor tissue. In some embodiments, the solid cancer is a brain cancer. In some embodiments, the brain cancer has high tumor endothelin-A receptor expression relative to non-tumor tissue. In some embodiments, the solid tumor is melanoma. In some embodiments, the solid tumor is renal cell carcinoma. In some embodiments, the solid tumor is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the solid tumor is hepatocellular carcinoma. In some embodiments, the solid tumor is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor is squamous cell carcinoma of the head and neck. In some embodiments, the solid tumor is urothelial carcinoma. In some embodiments, the solid tumor is esophageal squamous cell carcinoma. In some embodiments, the solid tumor is gastric cancer. In some embodiments, the solid tumor is esophageal cancer. In some embodiments, the solid tumor is cervical cancer. In some embodiments, the solid tumor is Merkel cell carcinoma. In some embodiments, the solid tumor is endometrial carcinoma. In some embodiments, the solid tumor is cutaneous squamous cell carcinoma. In some embodiments, the solid tumor is a cancer that has compressed blood vessels and/or is hypoperfused. In some embodiments, the solid tumor is a cancer that has compressed blood vessels. In some embodiments, the solid tumor is a cancer that is hypoperfused. In some embodiments, the solid tumor that has compressed blood vessels and/or is hypoperfused is selected from the group consisting of breast cancer, breast cancer lung metastases, pancreatic cancer, ovarian cancer, and liver metastases. In some embodiments, the solid tumor that has compressed blood vessels and/or is hypoperfused is breast cancer. In some embodiments, the breast cancer has high tumor endothelin-B receptor expression relative to non-tumor tissue. In some embodiments, the breast cancer is triple negative breast cancer. In some embodiments, the solid tumor that has compressed blood vessels and/or is hypoperfused is pancreatic cancer. In some embodiments, the solid tumor that has compressed blood vessels and/or is hypoperfused is ovarian cancer. In some embodiments, the solid tumor that has compressed blood vessels and/or is hypoperfused is a liver metastasis. In some embodiments, the liver metastasis is from colorectal cancer. In some embodiments, the solid tumor that has compressed blood vessels and/or is hypoperfused is a lung metastasis. In some embodiments, the liver metastasis is from breast cancer. In some embodiments, the solid tumor is a cancer that has endothelin receptor expression in the tumor vasculature and/or fibroblasts. In some embodiments, the solid tumor is a cancer that has endothelin receptor expression in the tumor vasculature. In some embodiments, the solid tumor is a cancer that has endothelin receptor expression in the tumor fibroblasts. In some embodiments, the solid tumor that has endothelin receptor expression in the tumor vasculature and/or fibroblasts is selected from the group consisting of pancreatic cancer, ovarian cancer, lung cancer, prostate cancer, brain cancer, breast cancer, and colorectal cancer. In some embodiments, the solid tumor that has endothelin receptor expression in the tumor vasculature and/or fibroblasts is pancreatic cancer. In some embodiments, the solid tumor that has endothelin receptor expression in the tumor vasculature and/or fibroblasts is ovarian cancer. In some embodiments, the solid tumor that has endothelin receptor expression in the tumor vasculature and/or fibroblasts is lung cancer. In some embodiments, the lung cancer expresses endothelin-A receptor. In some embodiments, the lung cancer expresses endothelin-B receptor. In some embodiments, the lung cancer expresses both endothelin-A receptor and endothelin-B receptor. In some embodiments, the lung cancer has high tumor endothelin-A receptor expression relative to non-tumor tissue. In some embodiments, the lung cancer has high tumor endothelin-B receptor expression relative to non-tumor tissue. In some embodiments, the lung cancer has high tumor endothelin-A receptor and endothelin-B receptor expression relative to non-tumor tissue. In some embodiments, the lung cancer is non-small cell lung cancer. In some embodiments, the lung cancer is small cell lung cancer. In some embodiments, the solid tumor that has endothelin receptor expression in the tumor vasculature and/or fibroblasts is prostate cancer. In some embodiments, the prostate cancer has high tumor endothelin-A receptor expression relative to non-tumor tissue. In some embodiments, the solid tumor that has endothelin receptor expression in the tumor vasculature and/or fibroblasts is brain cancer. In some embodiments, the brain cancer has high tumor endothelin-A receptor expression relative to non-tumor tissue. In some embodiments, the solid tumor that has endothelin receptor expression in the tumor vasculature and/or fibroblasts is breast cancer. In some embodiments, the breast cancer has high tumor endothelin-B receptor expression relative to non-tumor tissue. In some embodiments, the breast cancer is triple negative breast cancer. In some embodiments, the solid tumor is a lung metastasis from breast cancer. In some embodiments, the solid tumor that has endothelin receptor expression in the tumor vasculature and/or fibroblasts is colorectal cancer. In some embodiments, the colorectal cancer has high tumor endothelin-A receptor expression relative to non-tumor tissue. In some embodiments, the colorectal cancer has low tumor endothelin-B receptor expression relative to non-tumor tissue. In some embodiments, the colorectal cancer has high tumor endothelin-A receptor expression and low endothelin-B receptor expression relative to non-tumor tissue. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for intraperitoneal injection, subcutaneous, intravenous (e.g., intravenous infusion), or other modes of administration for treating cancer (e.g., a solid tumor) in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein ketotifen, or a pharmaceutically acceptable salt thereof, as described herein is a first medicament, and which article or kit further comprises instructions on the label or package insert for treating the subject with the second medicament, in an effective amount. In some embodiments, the second medicament is a checkpoint inhibitor as described herein. In some embodiments, the label or package insert indicates that the first and second medicaments are to be administered sequentially or simultaneously, as described herein.

In some embodiments, an ketotifen as described herein and/or a chemotherapeutic agent as described herein is present in the container as a lyophilized powder. In some embodiments, ketotifen, or a pharmaceutically acceptable salt thereof, as described herein and/or a checkpoint inhibitor as described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Ketotifen Inhibits Angiogenesis and Restores Vascular Perfusion within the Tumor Stroma in a Dose Dependent Manner

This example shows that ketotifen intervenes with angiogenic signaling, allowing vascular normalization in two mouse models of fibrosarcoma and osteosarcoma.

### Cell Culture and Animal Tumor Models

Two mouse models of distinct sarcoma subtypes, fibrosarcoma (MCA205 cells) and osteosarcoma (K7M2wt cells), were used in this study.

MCA205, a mouse fibrosarcoma cell line (SCC173, Millipore), was cultured in RPMI-1640 expansion medium containing 2 mM L-glutamine, 1 mM sodium pyruvate, 10% fetal bovine serum, 1× non-essential amino acids (TMS-001-C, Sigma), 1% antibiotics (A5955, Sigma), and 1×β-mercaptoethanol. K7M2wt, a mouse osteosarcoma cell line (CRL2836™, ATCC®), was cultured in DMEM expansion medium supplemented with 10% FBS and 1% antibiotics. All cells were maintained at 37° C./5% CO₂.

A fibrosarcoma syngeneic tumor model was generated by subcutaneous implantation of 2.5×10⁵ MCA205 cells in 50 μL of serum-free medium into the flank of 6-week old C57BL/6 female mice. A osteosarcoma syngeneic tumor model was generated by implanting K7M2wt tumor chunks into the fat pad of 6-week old BALB/c female mice. All mice were maintained in specific pathogen-free conditions and housed in controlled temperature/humidity (22° C./55%) environment on a 12-hour light-dark cycle and kept with free access to food and water throughout experiment period.

### Ketotifen Treatment and Tumor Growth Evaluation

Each treatment group of 4-7 mice received subtherapeutic doses of ketotifen (1, 5, 10 and 25 mg/kg) for a period of 8 days. Ketotifen fumarate salt was dissolved in sterilized normal saline (0.9% NaCl in ddH₂O, w/v). Ketotifen 1 mg/kg, 5 mg/kg, 10 mg/kg, 25 mg/kg or equal volume of diluent (control group) was administered by intraperitoneal injection (i.p.) once a day for 8 days. Ketotifen treatment for MCA205 and K7M2wt tumor models started when the average tumor volume reached 60 mm³ and 150 mm³, respectively, and completed at 600 mm³. At study endpoint, primary tumors were surgically excised and stored in 1×PBS at −80° C. until further processing. Planar dimensions (x, y) of tumor were monitored every 2-3 days using a digital caliper and tumor volume was estimated from the volume of an ellipsoid and assuming that the third dimension, z, is equal to sqrt(x y).

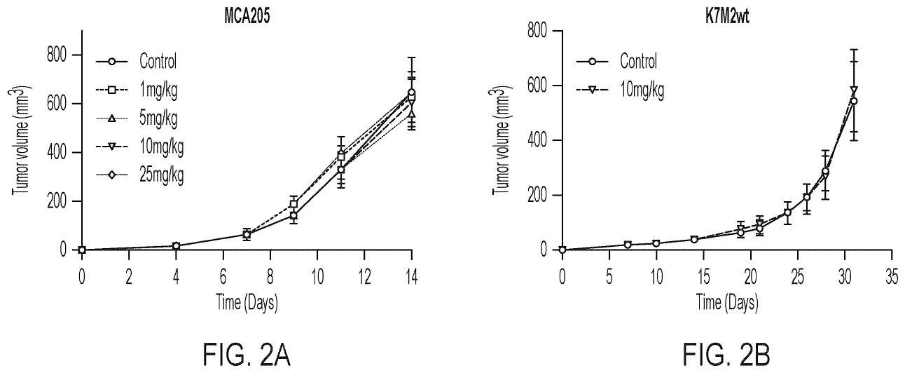

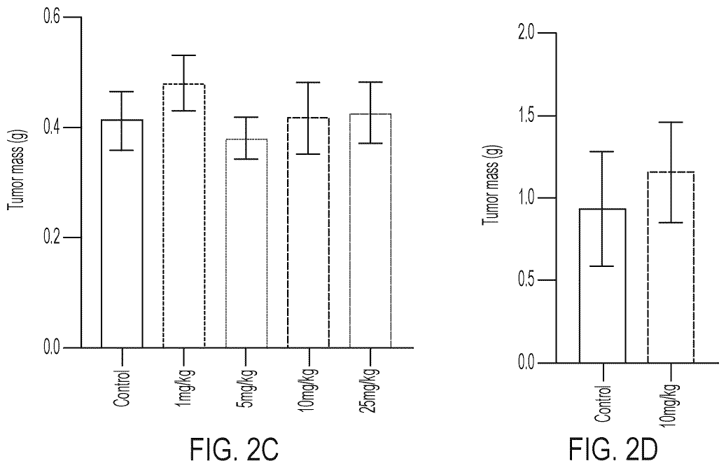

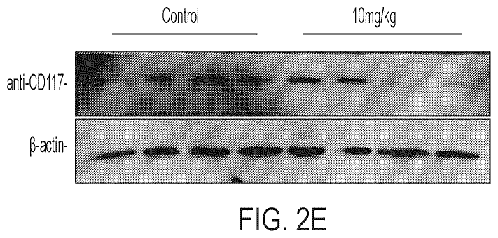

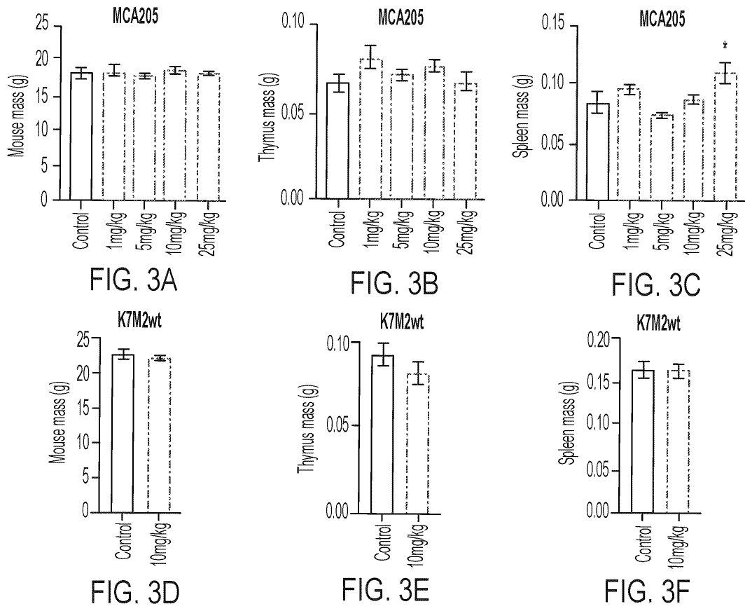

Following treatment, tumor growth (tumor volume and mass, see, FIGS. 2A and 2C for MCA205 tumors, and FIGS. 2B and 2D for K7M2wt tumors) was evaluated in the mice of each sarcoma group. No subtherepautic dose of ketotifen exhibited an antitumor effect in either mouse sarcoma model (FIGS. 2A-2D). Further, no subtherepautic dose of ketotifen impacted mast cell (MC) number as determined by CD117 protein levels, which remain unaffected by ketotifen treatment (FIG. 2E). Mouse body, thymus and spleen weight measurements at the conclusion of study indicated that a 10 mg/kg dose of ketotifen was tolerable (FIGS. 3A-3C for mice bearing MCA205 tumors; FIGS. 3D-3F for mice bearing K7M2wt tumors).

### Hypoxia Studies

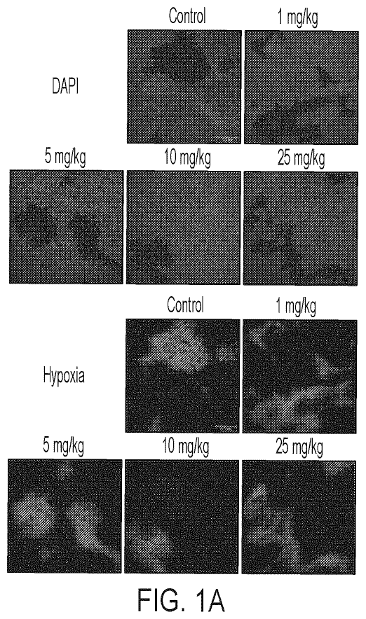

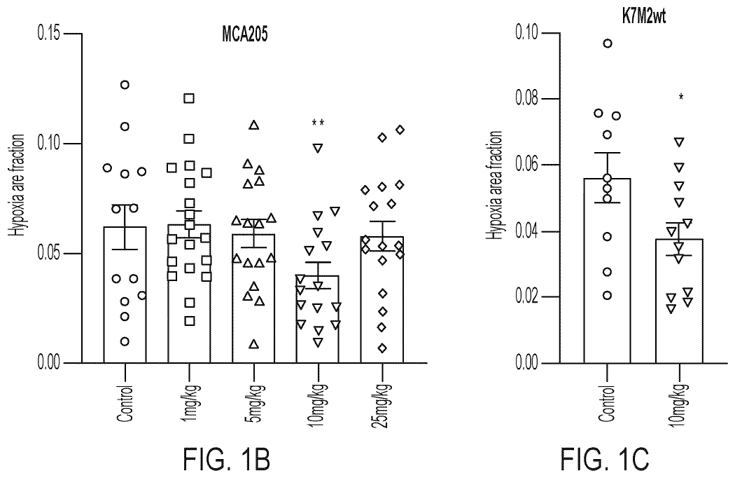

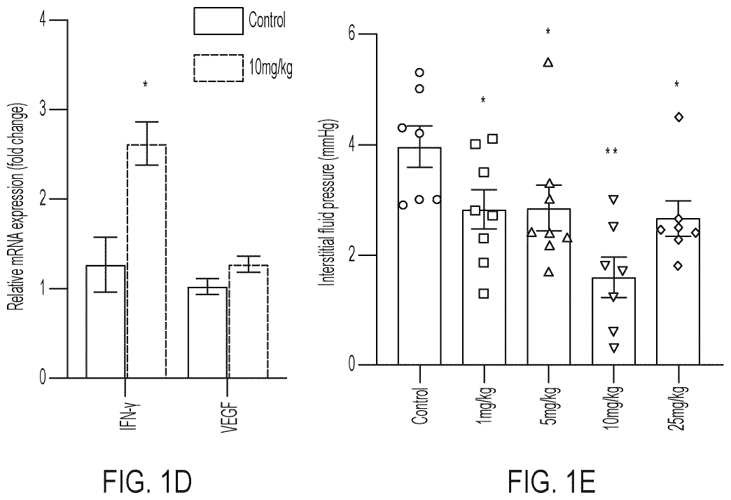

Mice bearing orthotopic MCA205 or K7M2wt sarcoma tumors were injected (intraperitoneal) with 60 mg/kg of pimonidazole HCl at 2 hours prior to tumor removal. Tumors were removed, washed twice in 1×PBS for 10 minutes and incubated with 4% PFA overnight at 4° C. The fixative was aspirated, and samples were washed twice in 1×PBS for 10 min. Tissue samples were dehydrated in successive ethanol steps and xylene followed by paraffin embedding. Serial sections (7 m) of paraffin-embedded tissues were produced using the microtome (Accu-Cut SRM 200 Rotary Microtome, SAKURA), flattened out into water, and allowed to dry overnight at 37° C. Sections were then deparaffinized and rehydrated as routinely processed for histology. Slides were microwaved in TriSodium Citrate, blocked in serum solution, and incubated with mouse anti-pimonidazole RED 549 conjugate antibody (HP7-100Kit, 1:100) overnight at 4° C. The next day slides were washed, nuclei were stained with DAPI, and tissue sections were mounted. Immunofluorescence images of the slides were taken (FIG. 1A), and hypoxic area fraction across different treatment groups was normalized to DAPI staining.

Pimonidazole positive fraction was prominent across untreated, 1 mg/kg, 5 mg/kg, and 25 mg/kg ketotifen treated mice, and significantly reduced in mice receiving the 10 mg/kg ketotifen dose (FIG. 1A-1C). The intratumoral levels of hypoxia were determined by quantifying the fluorescence signal of pimonidazole adducts in the images normalized to DAPI in both MCA205 (FIG. 1B) and K7M2wt tumors (FIG. 1C).

mRNA Expression Levels

Total RNA was isolated from breast tumors according to the standard Trizol-based protocol (Invitrogen), and cDNA synthesis was performed using reverse transcriptase III (RT-III) enzyme and random hexamers (Invitrogen). Real-time polymerase chain reaction was performed using Sybr Fast Universal Master Mix (KAPA). The specific mouse primers used for gene expression analysis of IFN-γ, VEGF, and B-actin control are listed in Table 1, below. Reactions were performed using a CFX-96 real-time PCR detection system (BioRad) at the following conditions: 95° C. for 2 minutes, 95° C. for 2 seconds, 60° C. for 20 seconds, 60° C. for 1 second, steps 2-4 for 39 cycles. Real-time PCR analysis and calculation of changes in gene expression between compared groups was performed using the ΔΔ^{Ct} method. Relative gene expression was normalized based on the expression of β-actin. For qPCR analysis, 3-5 biological samples were used per treatment and 3 technical replicates for each sample.

TABLE 1
qPCR Primer Sequences

| | Forward sequence | Reverse Sequence |
|---|---|---|
| IFN-γ | ATGAACGCTACACACTGCATC | CCATCCTTTTGCCAGTTCCTC |
| | (SEQ ID NO: 1) | (SEQ ID NO: 2) |
| VEGF | AGCACAGCAGATGTGAATGC | TTTCTTGCGCTTTCGTTTTT |
| | (SEQ ID NO: 3) | (SEQ ID NO: 4) |
| B- | GACGGCCAGGTCATCACTAT | AAGGAAGGCTGGAAAAGAGC |
| actin | (SEQ ID NO:5) | (SEQ ID NO:6) |

Ketotifen upregulated the mRNA levels of angiostatic gene IFN-γ without affecting the mRNA levels of common angiogenic factors like VEGF (FIG. 1D).

### Interstitial Fluid Pressure

Interstitial fluid pressure (IFP) was measured in vivo using the “wick-in-needle” technique after mice were anesthetized with i.p. injection of Avertin and prior to tumor excision. Additional information regarding the wick-in-needle technique is described in Dong et al., Involvement of mast cell chymase in burn wound healing in hamsters 2013; 5:643-7 and Shankaran et al. IFNγ and lymphocytes prevent primary tumour development and shape tumour immunogenicity 2001; 410:1107-11, the contents of which are incorporated herein by reference in their entirety.

All doses of ketotifen reduces the IFP, with the 10 mg/kg dose exhibiting the greatest effect (FIG. 1E), further underlining its potential to prevent fluid leakage into the tumor microenvironment (TME), consequent to the re-establishment of vascular functionality.

Overall, these results indicate that ketotifen inhibits angiogenesis and restores vascular perfusion within the tumor stroma in a dose dependent manner, with 10 mg/kg ketotifen dosage resulting in a strong effect.

### Example 2. Ketotifen Alleviates Intratumoral Stiffness by Inhibiting Extracellular Matrix Formation

This example shows that ketotifen facilitates vascular decompression, which is a measure of intratumoral stiffness, by inducing stress alleviation within the TME of mouse sarcoma models.

### Ultrasound Elastography Measurements

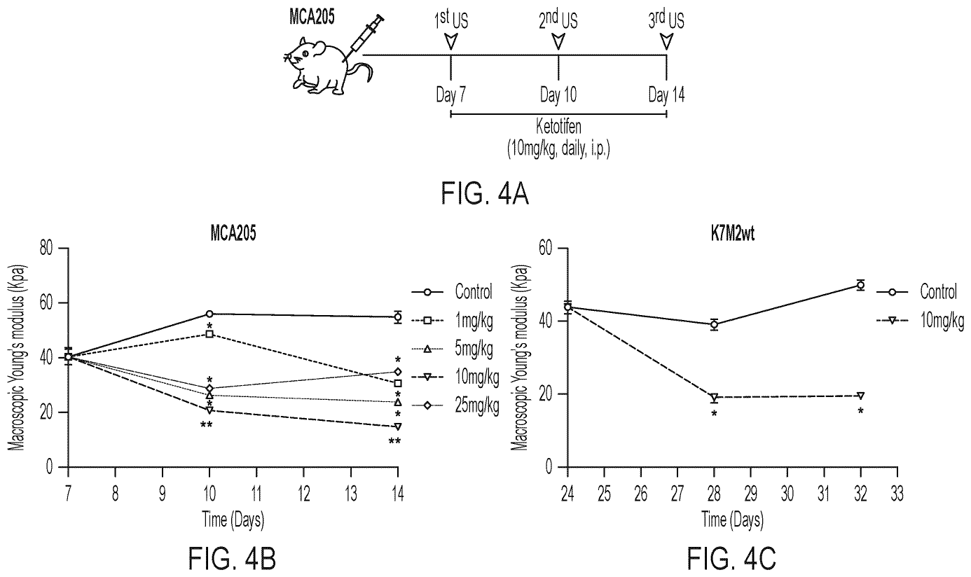

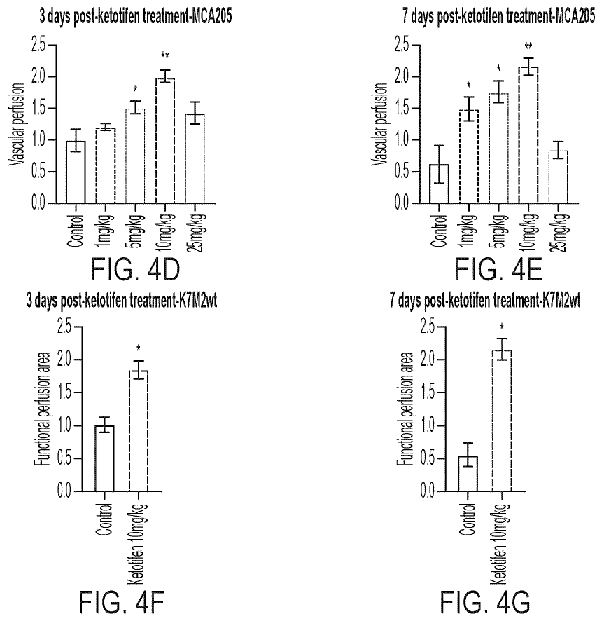

Cell culture and animal sarcoma tumor models were prepared as described above. A study outline is shown in FIG. 4A.

The mechanotherapeutic potential of ketotifen in reducing matrix stiffness was primarily assessed non-invasively and longitudinally using ultrasound elastography. Briefly, assessment of tumor elastic modulus was elastic via shear wave elastography using a Philips EPIQ Elite Ultrasound scanner with an eL18-4 linear array, approved for clinical scanning was performed as described in previous study. Elastic modulus values of the areas of the region of interest (ROI) with the highest shear wave quality were obtained. The elastic modulus value presented is the average within the tumor region. Shear wave imaging of MCA205 tumors was performed prior to ketotifen treatment on day 7, to determine baseline level of tumor stiffness, after three days of treatment (day 11), and on the last day of treatment just before tumor removal (day 15). Ultrasound imaging of K7M2wt tumors was performed on day 24, 28, and 31. The effect of ketotifen on chemo-immunotherapy combination with regards to MCA205 tissue elasticity was assessed on day 7 before the first cycle of doxorubicin+anti-PD-L1 treatment, one day after second cycle (day 11) and two days after the last cycle (day 15). Ultrasound imaging of K7M2wt tumors was performed on day 22 (i.e., four days post daily ketotifen and before first cycle of combination treatment) and upon completion of treatment (day 32).

A dose of 10 mg/kg ketotifen reduced tissue stiffness the most in mice bearing MCA205 fibrosarcoma tumors, with Young's modulus values reaching the 20 kPa, resembling the elasticity of healthy tissue (FIG. 4B). K7M2wt osteosarcoma tumors were similarly benefited from 10 mg/kg of daily ketotifen (FIG. 4C). The microscopic elasticity measurements obtained by AFM agreed with the macroscopic data.

### Vascular and Functional Perfusion

Vascular perfusion and functional perfusion were measured simultaneously during the course of ketotifen treatment, at day 3 and day 7, using contrast-enhanced ultrasound in mice bearing MCA205 and mice bearing K7M2wt tumors. Tumor perfusion was assessed after bolus injection of 8 μl SonoVue/Lumason contrast agent (Bracco Diagnostics, Geneva, Switzerland). SonoVue contains sulphur hexafluoride microbubbles encapsulated with a phosphoilipid shell and having a mean diameter of 2.5 μm. It is delivered as a retro-orbital injection since tail vein microbubble injections tend to have high variability. Prior to each ultrasound application, mice were anesthetized by i.p. injection of Avertin (200 mg/kg). As shown in FIGS. 4D-4G, the dose of 10 mg/kg ketotifen caused a significant increase in vascular and functional perfusion in both sarcoma subtypes.

### Collagen Staining

As tissue stiffness is caused in part by the degree of collagen expression by cancer associated fibroblasts (CAFs) and crosslinking, the intratumoral collagen levels were evaluated by histological staining using Picrosirius red.

Briefly, tumors were removed from four mice, washed twice in 1×PBS for 10 min, and incubated with 4% PFA overnight at 4° C. The fixative was aspirated, and samples were washed twice in 1×PBS for 10 min. Tissue samples were dehydrated in successive ethanol steps and xylene followed by paraffin embedding. Serial sections (7 m) of paraffin-embedded tissues were produced using the microtome (Accu-Cut SRM 200 Rotary Microtome, SAKURA), flattened out into water, and allowed to dry overnight at 37° C. Sections were then deparaffinized and rehydrated as routinely processed for histology. Following deparaffinization and rehydration, tissue sections were submerged in Picrosirius red stain for 1 hour at room temperature. Next, tissue sections were rinsed in two changes of acetic acid, followed by two changes of absolute ethanol and finally, mounted with DPX mountant for histology (Sigma). Collagen fibers were stained in red while the remaining tissue was pale yellow.

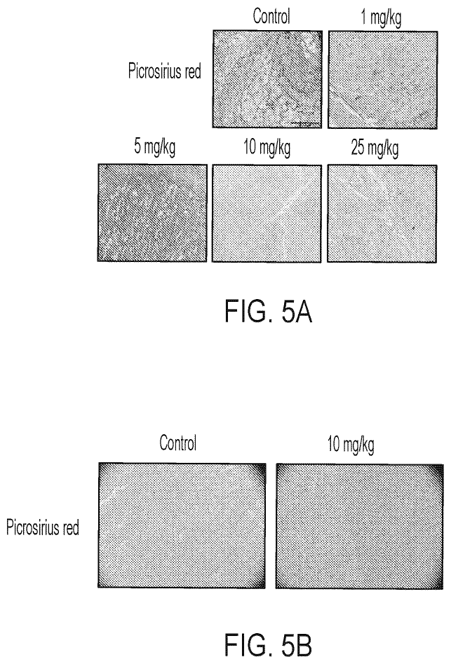

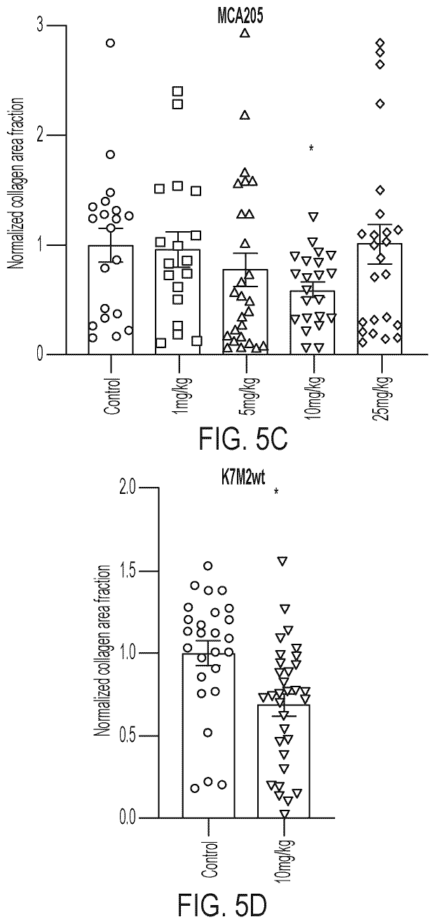

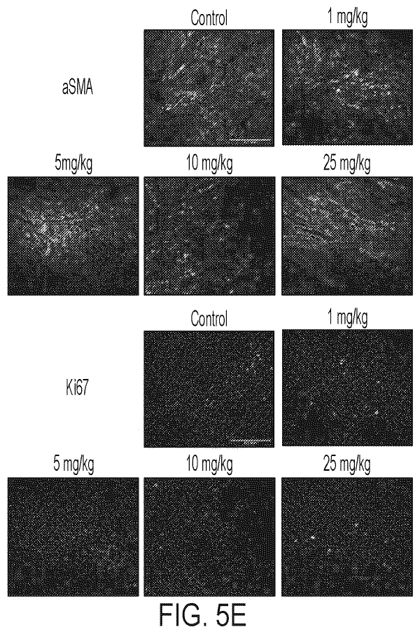

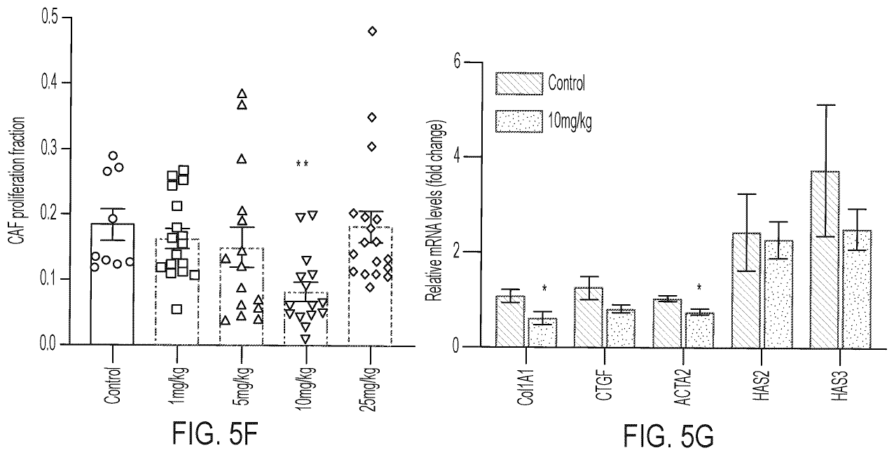

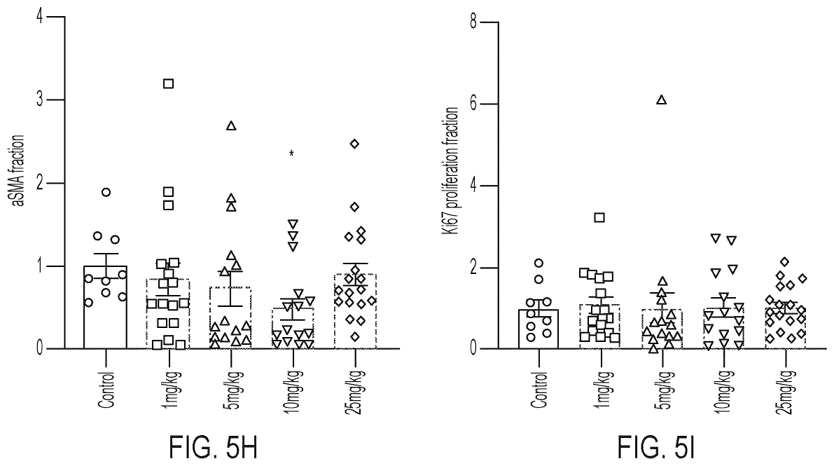

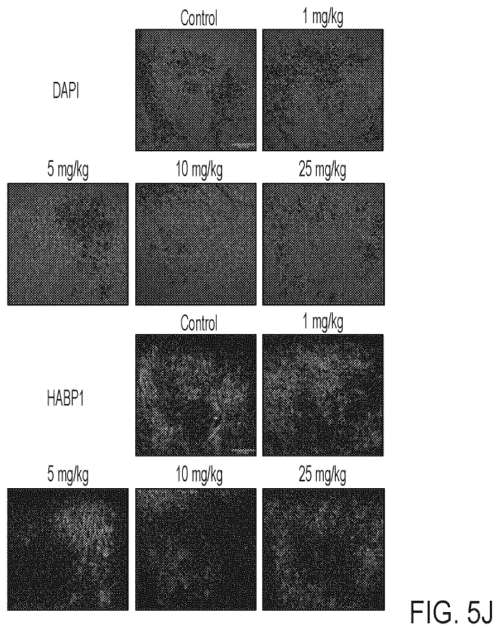

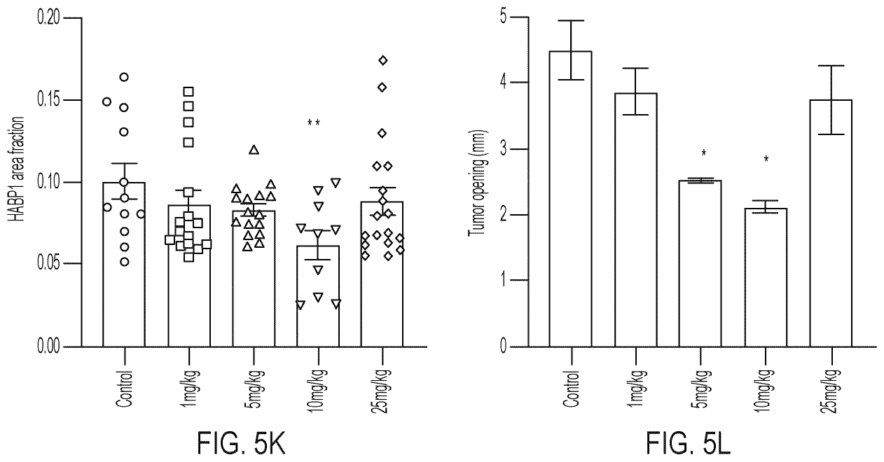

The dose of 10 mg/kg ketotifen caused a significant reduction in collagen deposition in both sarcoma subtypes (FIGS. 5A and 5C for MCA205 tumors; FIGS. 5B and 5D for K7M2wt tumors).

### Proliferation of CAFs

Proliferation of CAFs was evaluated by immunostaining for the presence of alpha-smooth muscle actin (α-SMA) (ab5694, Abcam; diluted 1:50) protein and Ki-67 (Abcam; diluted 1:200) proliferation marker following antigen retrieval, 1 hour incubation in serum solution (10% FBS, 3% donkey serum in 1×PBS), and 1 hour incubation in blocking solution for endogenous mouse IgG (Abcam; diluted 1:100). Following washes with TBS-T (0.25% Tween-20), Alexa Fluor-647 anti-rabbit IgG (H+L) (Invitrogen; diluted 1:400) and Fluor-488 anti-rat IgG (H+L) (Invitrogen; diluted 1:400) secondary antibodies and DAPR (Sigma diluted 1:100 of 1 mg/mL stock) were applied for 1 hour at room temperature. Sections were then mounted on microscope slides using the ProLong gold antifade mountant (Invitrogen) and covered with a glass coverslip. CAF proliferation fraction was detected as the ratio of overlapping α-SMA (green) and Ki67 (red) positive signal to total CAF signal (α-SMA⁺), indicating CAFs.

The alteration in the extracellular matrix (ECM) as indicated by reduction in collagen deposition following 10 mg/kg ketotifen treatment (FIGS. 5A-5D) was verified evaluating CAF levels and proliferation activity in FIGS. 5E-5I.

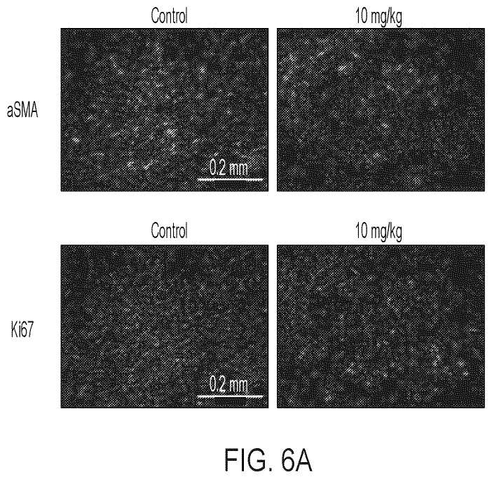

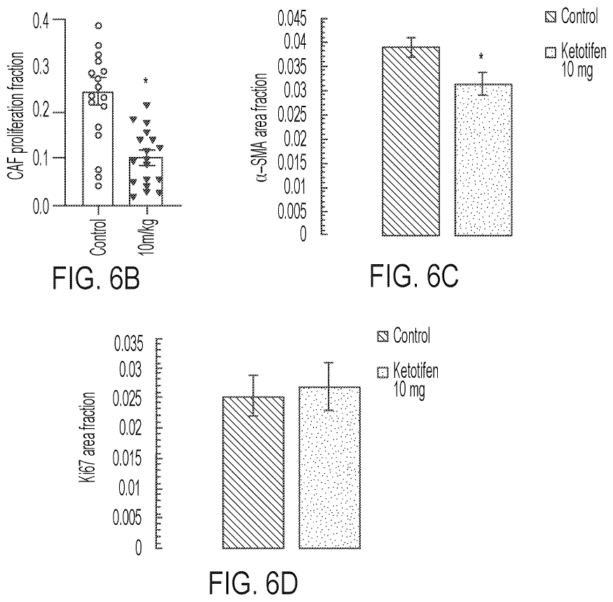

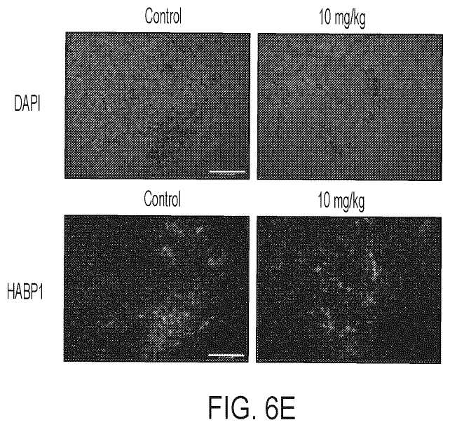

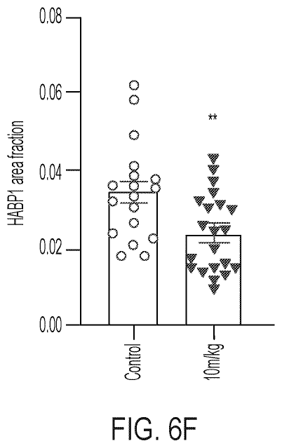

FIGS. 5H-5I and FIGS. 6C-6D show the fraction of area positive for α-SMA staining (FIGS. 5H and 6C) and quantification of the fraction of area positive for Ki-67 staining (FIGS. 5I and 6D) in mice bearing MCA205 tumors and mice bearing K7M2wt tumors in the representative immunofluorescence images of FIG. 5E and FIG. 6A, respectively. As indicated by immunofluorescence staining of α-SMA and Ki-67, respectively, in fibrosarcoma tumors (FIGS. 5E-5F and FIGS. 6A-6B), 10 mg/kg ketotifen treatment results in a decrease in CAF levels and proliferation activity in both mouse sarcoma models.

mRNA Expression Levels

mRNA expression levels were evaluated in both mouse sarcoma models by qPCR, as described above. The specific mouse primers used for gene expression analysis are listed in Table 2.

TABLE 2
Additional qPCR primer sequences.

| | Forward seq. | Reverse seq. |
|---|---|---|
| Col1a1 | GAGCGGAGAGTACTGGATCG | GTTCGGGCTGATGTACCAGT |
| | (SEQ ID NO: 7) | (SEQ ID NO: 8) |
| Ctgf | CACTCTCCAGTGGAGTTCA | GTAATGGCAGGCACAGGTCT |
| | (SEQ ID NO: 9) | (SEQ ID NO: 10) |
| Acta2 | TGTGCTGGACTCTGGAGATG | GAAGGAATAGCCACGCTCAG |
| | (SEQ ID NO: 11) | (SEQ ID NO: 12) |
| Has2 | ATAAGCGGTCCTCTGGGAAT | CCTGTTGGTAAGGTGCCTGT |
| | (SEQ ID NO: 13) | (SEQ ID NO: 14) |
| Has3 | TTCCAAACCTCAAGGTGGTC | TGCTACGCCACACAAAGAAG |
| | (SEQ ID NO: 15) | (SEQ ID NO: 16) |
| B-actin | GACGGCCAGGTCATCACTAT | AAGGAAGGCTGGAAAAGAGC |
| | (SEQ ID NO: 17) | (SEQ ID NO: 18) |

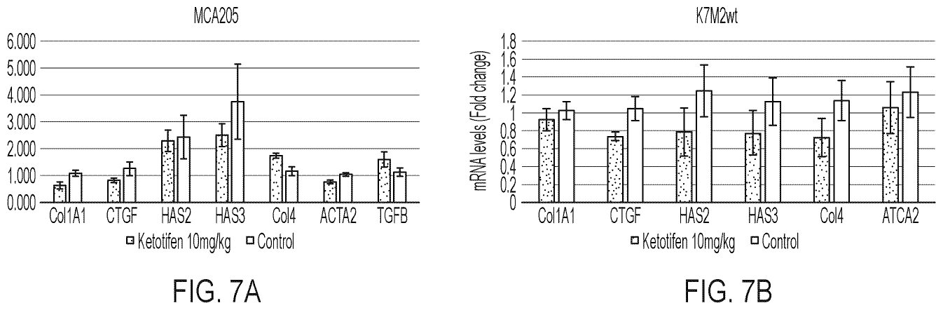

Ketotifen reduced the mRNA levels of collagen I encoding gene (Col1A1) and connective tissue growth factor (CTGR), although not to a statistically significant extent, but had no effect on hyaluronan synthase 2 (Has2) and hyaluronan synthase 3 (Has3) in MCA205 tumors (FIG. 5G and FIG. 7A). A similar effect was observed in K7M2wt tumors (FIG. 7B).

### Hyaluronan Evaluation

Besides collagen, other structural components of tumor stroma can exert pressure and deform vessels. Such an example is the hyaluronan (e.g., hyaluronic acid), which binds to and retains excessive fluid generating gel-like regions within the tissue. Therefore, hyaluronic acid staining was performed in both sarcoma mouse models.

Tumors were removed from mice, washed twice in 1×PBS for 10 min, and incubated with 4% PFA overnight at 4° C. The fixative was aspirated, and samples were washed twice in 1×PBS for 10 min. Tissue samples were dehydrated in successive ethanol steps and xylene followed by paraffin embedding. Serial sections (7 m) of paraffin-embedded tissues were produced using the microtome (Accu-Cut SRM 200 Rotary Microtome, SAKURA), flattened out into water, and allowed to dry overnight at 37° C. Sections were then deparaffinized and rehydrated as routinely processed for histology. Tissue sections were subjected to antigen retrieval (microwave heat treatment with TriSodium Citrate, pH 6, for 20 min), washed with 1×TBS/0.025% Triton X-100 (TBS-T), and incubated in blocking solution (2% BSA, 0.2% Triton 100×) for 2 hours at room temperature. Next, slides were incubated with biotinylated hyaluronan binding protein (b-HABP) (amsbio, 1:100) overnight at 4° C. Hyaluronan was detected following incubation with streptavidin-FITC conjugate (Invitrogen, 1:1000) and nuclei were stained with DAPI stain at room temperature for 1 hour in the dark. The area fraction positive for hyaluronan across various treatments was normalized to DAPI stain.

Immunofluorescence staining of hyaluronan binding protein 1 (HABP1) showed that administration of daily ketotifen at a 10 mg/kg dose reduced the expression of hyaluronan protein (FIGS. 5J-5K and 6E-F).

### Tumor Opening

Tumor-opening experiments were performed as an indicator of the amount of residual stress contained within the tissue, using the values of opening after cutting. MCA205 tumors treated with the moderate doses of ketotifen (5 mg/kg and 10 mg/kg), exhibited a smaller opening, suggesting reduced solid stress (FIG. 5L).

Overall, these results indicate that ketotifen (10 mg/kg) alleviates intratumoral stiffness in sarcoma tumors, and daily ketotifen induces ECM remodeling by inhibiting collagen and hyaluronan formation.

### Example 3. Ketotifen Pretreatment Enhances the Antitumor Effects of Immunotherapy by Promoting T Cell Recruitment and Cytotoxic Immune Responses

This example shows that ketotifen enhances the antitumor immune response in combination treatment. In particular, this example shows that a 10 mg/kg daily regimen of ketotifen enhances the antitumor immune response of doxorubicin/anti-PD-L1 combination treatment.

### Animal Tumor Models

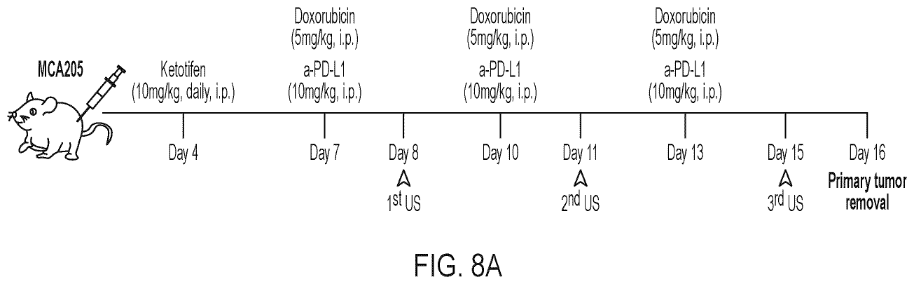

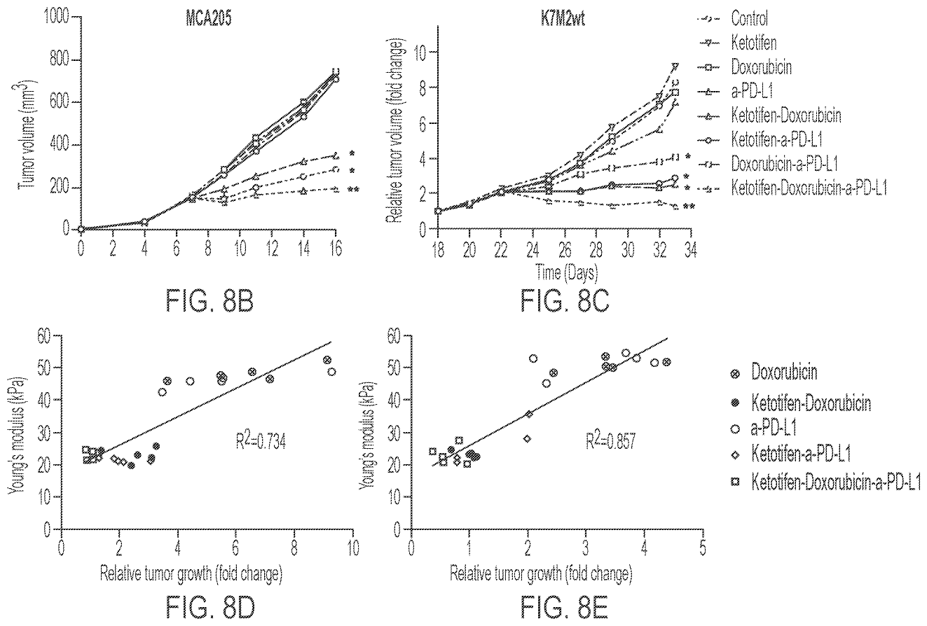

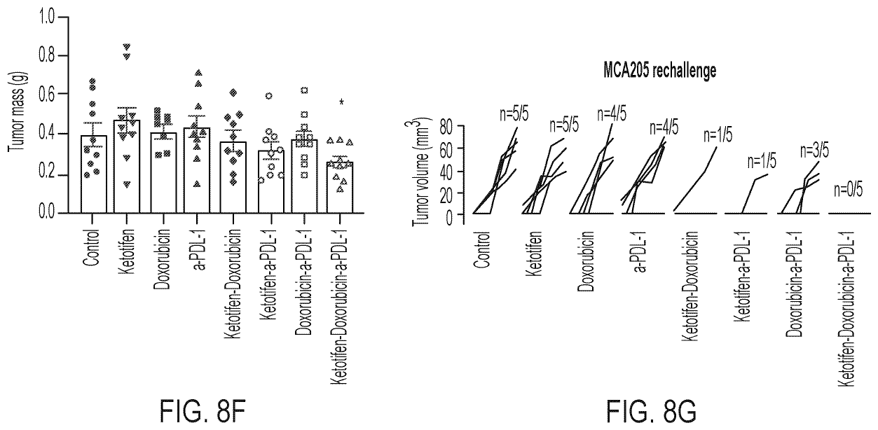

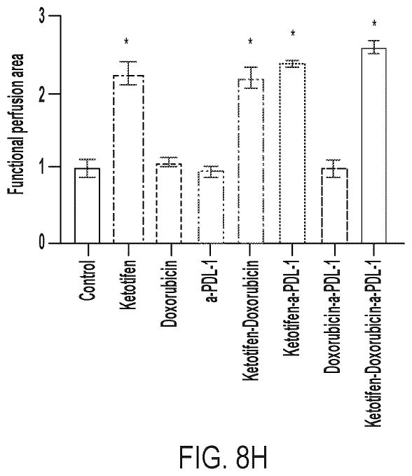

Mice bearing sarcoma tumors were pre-treated with 10 mg/kg daily ketotifen followed by three or four doses of the neoadjuvant chemotherapy combined with the immune checkpoint inhibitor (ICI) anti-PD-L1 antibody (FIG. 8A).

Fibrosarcoma syngeneic tumor models were generated, and ketotifen was prepared, as described above. Mouse monoclonal anti-PD-L1 (B7-H1, clone 10F.9G2, BioXCell) and rat IgG2b isotype control, anti-keyhole limpet hemocyanin (LTF-2, BioXCell) were dissolved in the recommended InVivoPure pH 7.0 Dilution Buffer (IP0070, BioXCell). Doxorubicin hydrochloride was obtained from Nicosia General Hospital as a ready-made solution of 2 mg/ml. Anti-PD-L1 was administered at a final dose of 10 mg/kg and doxorubicin at 5 mg/kg.

Mice bearing MCA205 tumors were pretreated with daily ketotifen 10 mg/kg or equal volume of diluent (control group) once the average tumor size reached 40 mm³, prior to the neoadjuvant treatment. Doxorubicin and anti-PD-L1 combination treatment initiated when tumors reached an average size of 150 mm³ (day 7) and was administered as i.p. injections every three days (day 7, 10, and 13) for three doses. Daily ketotifen continued until completion of doxorubicin-anti-PD-L1 combination treatment.

Primary tumors were resected and stored when they reached an average size of 700 mm³ on day 16 and mice were monitored for re-challenge experiments. Similarly, K7M2wt tumors were pretreated with daily ketotifen 10 mg/kg or equal volume of diluent (control group) once the average tumor size reached 70 or 80 mm³ (day 18) and continued until completion of neoadjuvant treatment. Doxorubicin and anti-PD-L1 combination treatment started when tumors reached an average size of 150 mm³ (day 22) and repeated on day 25, 28 and 31. Study ended when tumors reached a mean volume of 550 mm³ (day 33). Mice were sacrificed and tumors were collected for ex vivo analysis.

### Tumor Characterization

Neither anti-PD-L1 nor doxorubicin monotherapies affected tumor growth of fibrosarcoma MCA205 tumors, while their combination with ketotifen induced a significant antitumor response (FIG. 8B and FIG. 8F). In the osteosarcoma tumor model (K7M2wt), a similar reduction in the relative tumor growth was observed in mice receiving the doxorubicin-anti-PD-L1 combination, as well as, in all combinations of ketotifen mechanotherapeutic with ketotifen-doxorubicin and anti-PD-L1 triple combination causing the greatest attenuation of growth (FIG. 8C).

To support the hypothesis that the increased efficacy of cytotoxic treatment was attributed to ketotifen effects, the tissue stiffness prior to the first cycle of chemo- or immunotherapy in ketotifen and non-ketotifen treatment groups was measured and these values were correlated with the relative tumor volume, defined as the ratio of final volume to the volume obtained before the first cycle of chemo-immunotherapy treatment. The ketotifen-induced tissue stiffness alleviation was evaluated for correlation with tumor response to the neoadjuvant. Young's modulus measurements were determined as previously described, and recorded after 4 days of ketotifen administration. Young's modulus measurements positively correlate with relative tumor volumes across doxorubicin and anti-PD-L1 monotherapy and combination treatment groups in both MCA205 (FIG. 8D) and K7M2wt tumors (FIG. 8E). Response to therapy is improved when stiffness values drop below 30 kPa for both tumor models. These results show a linear, negative correlation between the elastic modulus measured at the beginning of cytotoxic treatment and the antitumor efficacy, as indicated by the R2 value of the best linear fit (FIG. 8D and FIG. 8E).

### Re-Challenge Experiment

Since doxorubicin has potential to establish favorable immunogenic conditions within the TME, which could affect the efficacy of immunotherapy, it was investigated if this immune response is preserved against rechallenge of long-term survivors with the same cell line. Mice were considered long-term survivors if no tumor was detected 80 days after surgical resection of MCA205 primary tumors. Five long-term survivors from each treatment group were further re-challenged with 2.5×10⁵ MCA205 cells injected subcutaneously into the flank region. Cells were prepared in 50 μl of PBS. Naïve mice were implanted in parallel as controls.

As shown in FIG. 8G, interestingly, no tumor growth occurred in ketotifen-doxorubiicin-anti-PD-L1 group, indicating a memory response upon tumor antigen recognition, i.e., the development of an adaptive memory response, which is triggered upon encountering of tumor antigens. On the contrary, all naïve and ketotifen-alone treated mice developed progressively growing tumors. Likewise, large tumors were formed in four mice of the doxorubicin and the anti-PD-L1 group, and in three mice of the doxorubicin-anti-PD-L1 group. In the ketotifen-doxorubicin and the ketotifen-anti-PD-L1 group, only one mouse out of five developed fibrosarcoma. Similar to the hierarchy of efficacy observed in tumor growth measurements, the combination of ketotifen-doxorubicin-anti-PD-L1 demonstrated therapeutic superiority to doxorubicin-anti-PD-L1 without ketotifen, offering a durable remission.

Taken together these findings demonstrate that pre-treatment with ketotifen is a pre-requisite to establishing favorable immunogenic conditions within the TME that are capable of strong anti-tumor effects that confers immunological memory and enable doxorubicin and anti-PD-L1 antibody treatment to confer immunological memory.

### Example 4. Ketotifen Alleviates Hypoxia and Restores Intratumoral T Cell Infiltration by Upregulating Immune Cell Adhesion to Blood Vessels

This example shows contribution of ketotifen and ketotifen combination treatment in stimulating immune responses.

### Flow Cytometry

On day 16 of the doxorubicin-anti-PD-L1 combination study (e.g., the study outlined in FIG. 8A), MCA205 tumors were harvested in 1×HBSS (Biosera), minced to fine fragments, and incubated with 1 mg/mL Collagenase D and 0.5 mg/ml DNase I (Roche) in a 37° C. shaker for 30 minutes. Enzymatic digestion was ceased by the addition of RPMI media containing 10% FBS and 1% antibiotic/antimycotic solution. The tissue homogenates were then filtered through 40 μm cell strainers. Red blood cells were removed by ACK Lysing buffer (A1049201, Gibco) and single cell suspensions were collected and used to a final concentration 1×10⁶ cells per sample. Cells were then incubated with fixable viability dye (eBioscience, 1:1000) for 10 minutes on ice for gating of viable cells. Non-specific antibody binding was blocked following incubation with the rat anti-mouse CD16/CD32 mAb (BD Bioscience) for 20 minutes on ice.

The anti-mouse antibodies used in the experiment were: CD45-V500 (BD Bioscience), CD3-PE (BD Bioscience), CD4-PerCP Cy5.5 (Invitrogen), CD25-PE-Cy7 (BD Bioscience), CD127-APC (BioLegend) and CD8a-e450 (eBioscience).

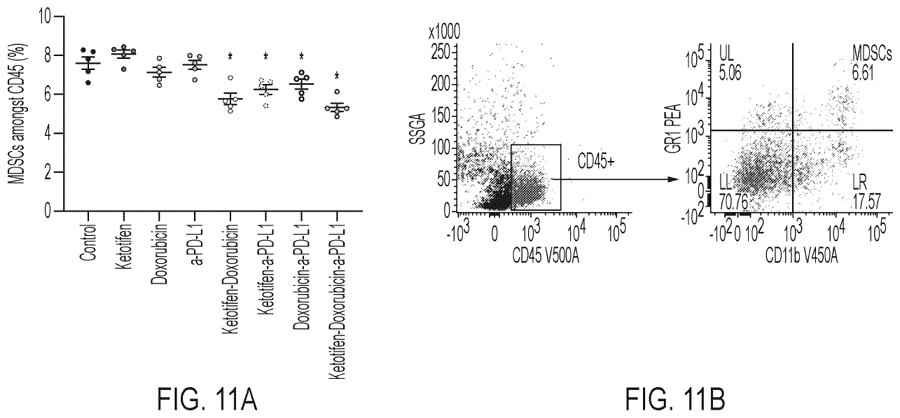

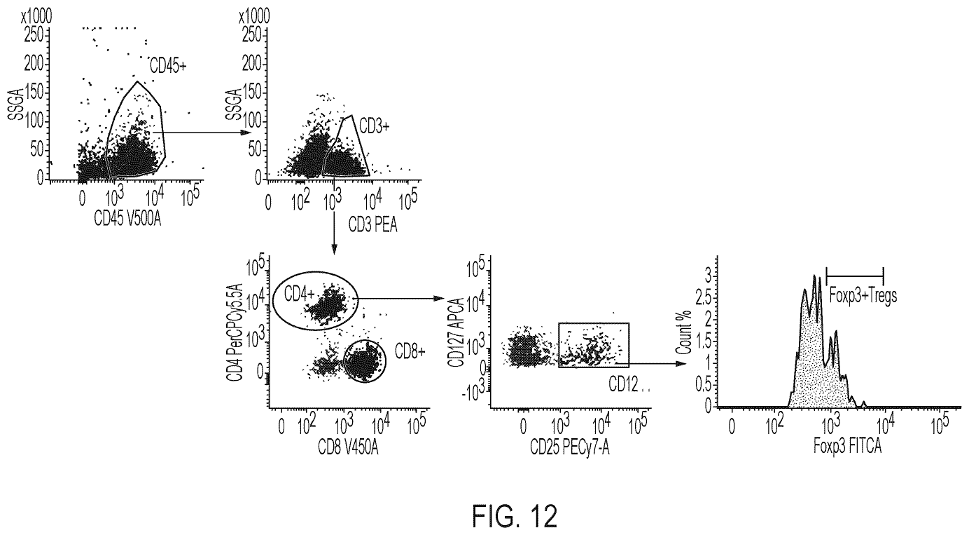

Foxp3-AF488 (BD Bioscience) staining was performed following fixation and permeabilization process using the Foxp3/Transcription Factor Staining Buffer Set (00-5523-00, eBioscience). For MDSCs staining the Gr-1-PE (Biolegend) and CD11b-e450 (eBiosceience) were used. Flow cytometry data were obtained using BD FACSLyric flow cytometer and analyzed using BD FACSuite™ Software. Data presented is representative of singlets, live cells. FIGS. 11A-11B and FIG. 12 provide details on the gating strategy for the flow cytometry experiments provided herein.

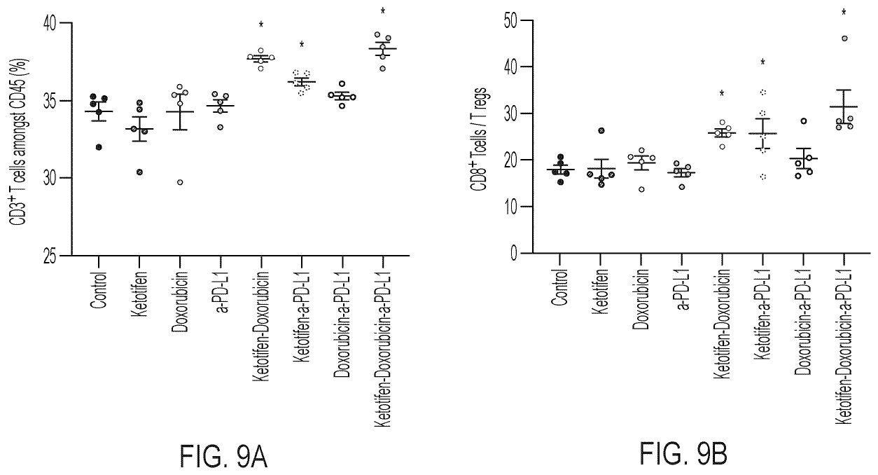

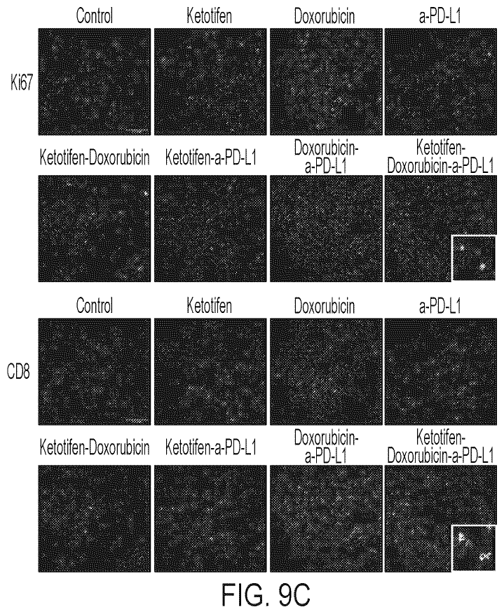

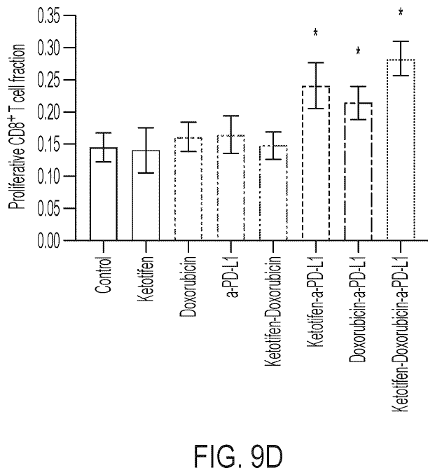

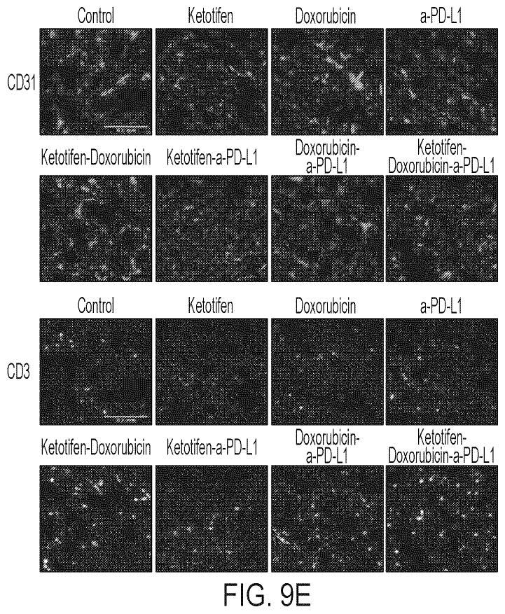

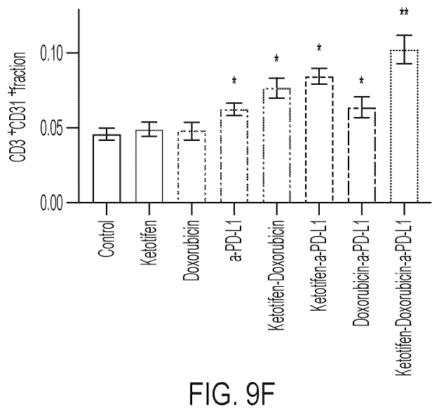

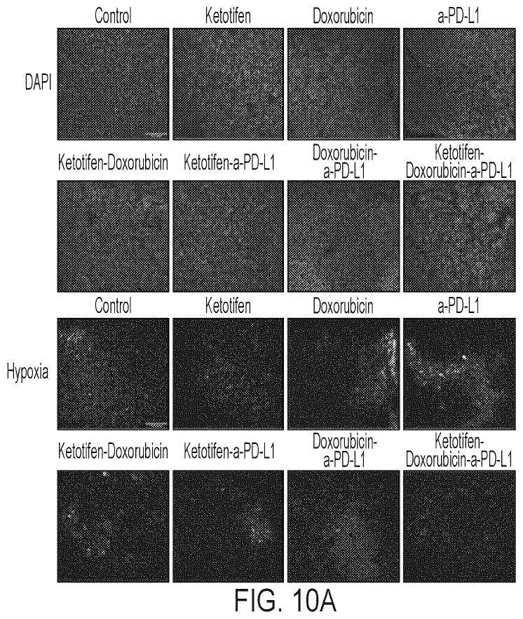

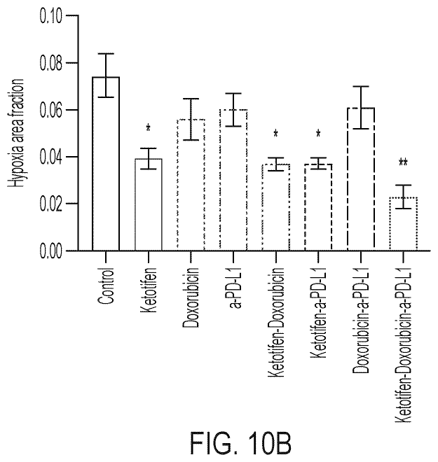

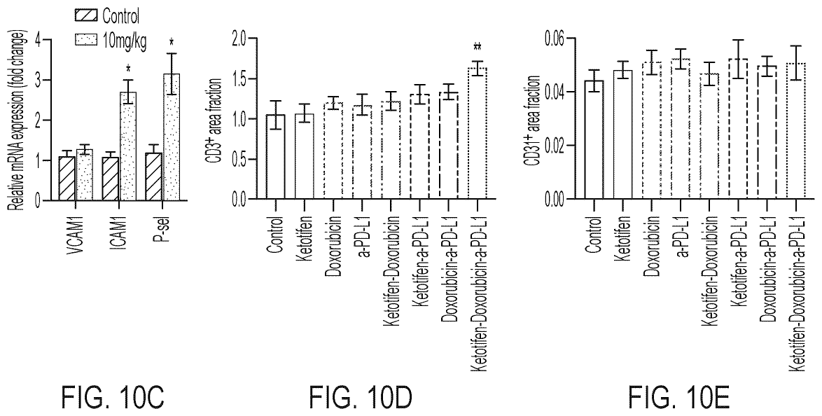

Total T cell population was primarily defined by the expression of CD3⁺ protein, a pan T cell marker, and then distinguished into the CD8⁺ cytotoxic or the regulatory T cell subtype (CD3⁺CD4⁺CD25^{hi}CD127^{lo}F.oxp3⁺) (FIG. 9A). There was statistically significant increase in total T cell recruitment of the doxorubicin-anti-PD-L1 and ketotifen-doxorubicin-anti-PD-L1 treatment groups. Although the magnitude of increase in CD8/T regs ratio is small compared to more immunogenic cancers like breast, it has been linked to response to ICIs. In K7M2wt tumors, however, only the groups of mice receiving ketotifen prior to the neoadjuvant regimen exhibited an increase in T cell infiltration as indicated by immunofluorescence staining for the CD3 protein (FIG. 10D). The ratio of CD8⁺ T cells to immunosuppressive T regs was increased after combining doxorubicin with either ketotifen or/and immunotherapy treatment (FIG. 9B).

Immunofluorescence and mRNA Expression

Immunofluorescence staining for Ki67 and CD8, performed as described above, confirmed the activation status of recruited CD8⁺ T cells in osteosarcoma tumors (K7M2wt tumors) (FIGS. 9C-9D). K7M2wt tissue sections were then stained for pimonidazole adducts as a measure of hypoxia formation. Only the groups receiving ketotifen exhibited a significant reduction of hypoxia area fraction, allowing adequate tumor oxygenation (FIG. 10A-10B).

RT-qPCR, as described above, was used to evaluate immune cell adhesion to the wall of perfused vessels. The specific mouse primers used for gene expression analysis are listed in Table 3.

TABLE 3
Additional qPCR primer sequences.

| | Forward seq. | Reverse seq. |
|---|---|---|
| Icam1 | CCTGTTTCCTGCCTCTGAAG | TTAAGGTCCTCTGCGTCTCC |
| | (SEQ ID NO: 19) | (SEQ ID NO: 20) |
| Vcam1 | CCCAGGTGGAGGTCTACTCA | CAGGATTTTGGGAGCTGGTA |
| | (SEQ ID NO: 21) | (SEQ ID NO: 22) |
| P-sel | GTCCACGGAGAGTTTGGTGT | AAGTGGTGTTCGGACCAAAG |
| | (SEQ ID NO: 23) | (SEQ ID NO: 24) |
| B- | GACGGCCAGGTCATCACTAT | AAGGAAGGCTGGAAAAGAGC |
| actin | (SEQ ID NO: 25) | (SEQ ID NO: 26) |

Ketotifen increased mRNA expression of the immune cell adhesion molecules ICAM and P-selectin (P-sel) (FIG. 10C).

Immunofluorescence staining of CD3 and CD31 endothelial marker was used to assess the presence of T cells in the proximity of tumor vessel capillaries. PFA-fixed tissue cryosections of K7M2wt osteosarcoma tumors were incubated with primary rabbit anti-CD31 (Abcam, 1:50) and rat anti-CD3 (BioLegend, 1:50) overnight at 4° C. CD31 signal was detected with Alexa Fluor-488 anti-rabbit IgG (H+L) (Invitrogen, 1:400) and CD3 signal with Alexa Fluor-647 anti-rat IgG (H+L) (Invitrogen, 1:400) secondary antibodies. Cell nuclei were stained with DAPI. Tumor associated T cell to vessel content was determined by the overlapping CD31 and CD3 area fraction normalized to DAPI staining. All combination treatments of ketotifen exhibit more association between T lymphocytes and endothelial cells, without effecting the endothelial cell area fraction (FIG. 10E). Anti-PD-L1 monotherapy and doxorubicin combination show an increased colocalization of CD3 T cells and CD31 (FIGS. 9E-9F).

Overall, these results demonstrate that ketotifen mechanotherapeutic enhances the neoadjuvant treatment by reducing the recruitment of immunosuppressive cell populations.

### Example 5. Further Studies of Ketotifen Effects

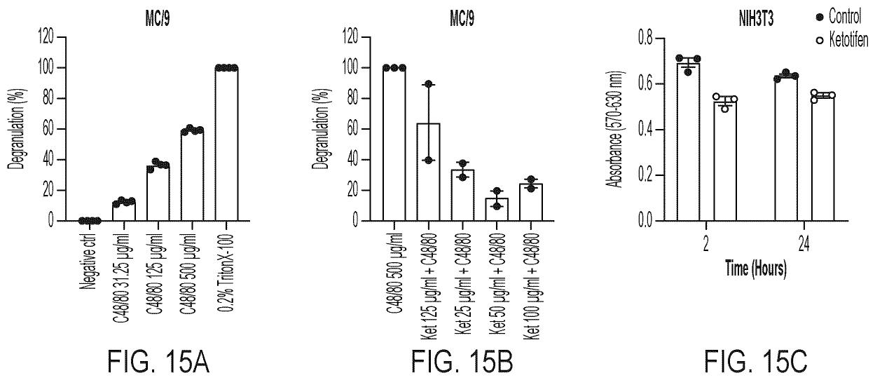

The presence of mast cells (MCs) in sarcoma tumors was documented by CD117 (c-Kit) immunofluorescence staining of human tissue microarrays. Mast cells were found in different sarcoma subtypes, indicating a role in modulating the tumor microenvironment (TME) and the existence of cancer-associated fibroblasts (see FIG. 16).

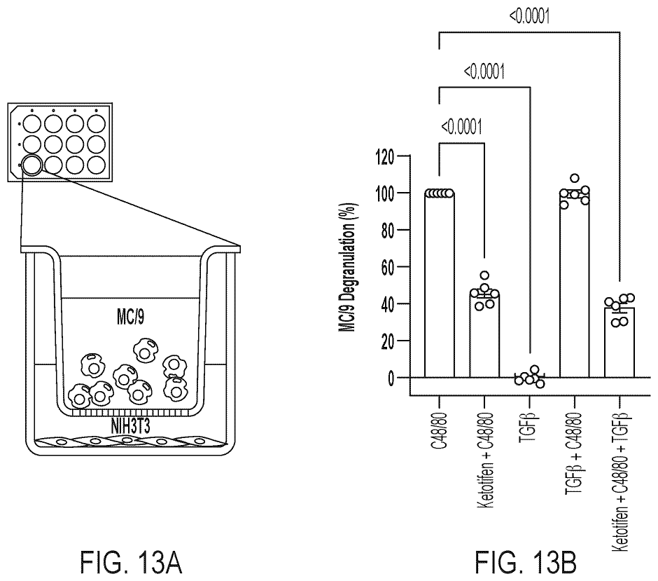

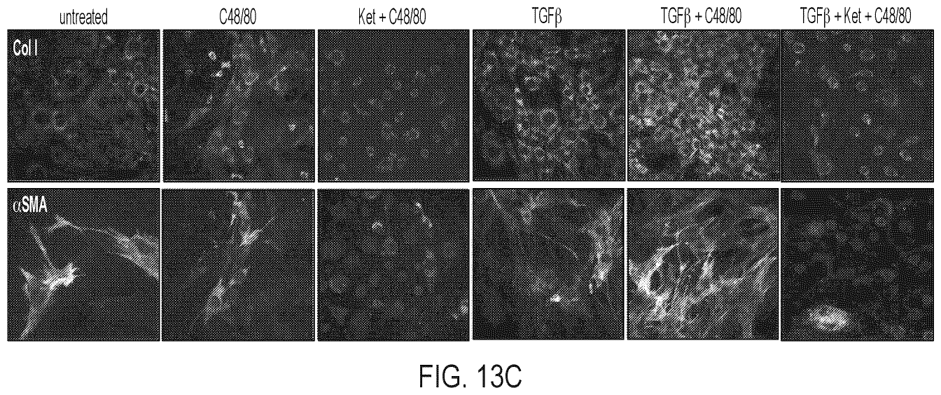

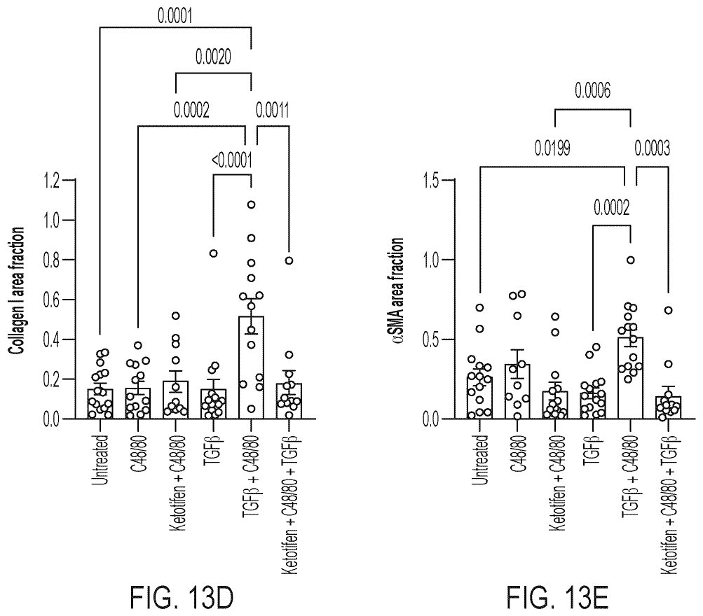

Given that both MCs and fibroblasts are present in the TME, the consequences of MC activation on myofibroblasts was explored using a co-culture assay in which murine MC/9 MCs and NIH3T3 myofibroblasts were separated by a transwell chamber with micropores that only allowed chemical communication (FIG. 13A). In this system, degranulation efficiency was determined by measuring the concentration of β-hexosaminidase (β-hex) in the supernatant. MC degranulation was induced through exposure to compound 48/80 (C48/80), which binds to the MRGPRB2 receptor (the orthologue of the human G-protein-coupled receptor MRGPRX2). As shown in FIG. 13B, ketotifen inhibited MC degranulation in this system. Co-cultures treated with a vehicle control and Triton X-100 were used as internal assay control to evaluate background and maximum β-hex release, respectively. Transforming growth factor (TGF)-β1 was used as a fibroblast activator due to its capacity to induce a myofibroblast phenotype of the NIH3T3 cells. MC/9 cells were cultivated with NIH3T3 cells for 24 hours and then treated with either ketotifen or TGFβ for additional 24 hours in low serum medium, followed by a 30 minute sensitization with C48/80. It was found that ketotifen inhibits degranulation in a dose-dependent manner, with the concentration of 100 μg/ml causing more than 50% reduction in 1-hex release compared to C48/80 treatment. The presence of TGFβ in the co-culture did not affect MC degranulation (see FIG. 13B, FIG. 15A, and FIG. 15B). Ketotifen treatment did not significantly reduce NIH3T3 cell viability (FIG. 15C).

To further explore the MC-fibroblast interaction, the differentiation process of NIH3T3 cells in the co-culture system was monitored by assaying levels of αSMA and collagen I, which are two markers of the myofibroblast phenotype, by immunofluorescence staining (FIG. 13C, FIG. 13D, and FIG. 13E). A significant increase in both αSMA and collagen I protein expression occurred only in the presence of both TGFβ and activated MCs, while no change was observed in the presence of TGFβ alone. Ketotifen impaired this myofibroblast differentiation. These in vitro results indicate that MCs assist the production of collagen by activated fibroblasts, whereas ketotifen inhibits this interaction.

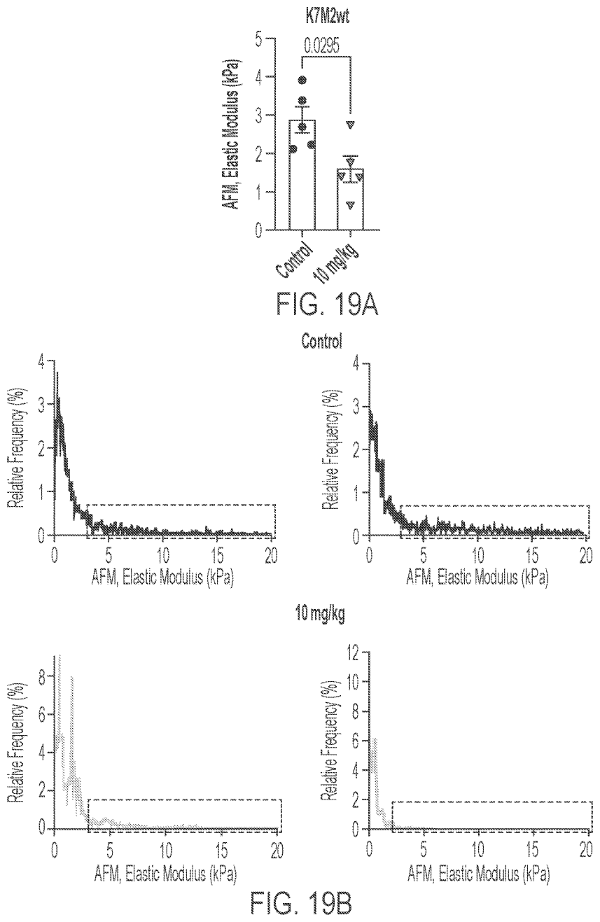

Following the confirmation of the presence of MCs in human sarcoma samples and the in vitro characterization of the MC-fibroblast interaction, it was assessed whether inhibition of MC degranulation would reduce fibrosis in murine sarcoma models. The impact of MC degranulation by treating MCA205 fibrosarcoma and K7M2wt osteosarcoma tumors with ketotifen was assessed. Ketotifen was administered in four different doses (1, 5, 10, and 25 mg/kg), once a day by i.p. injection for a period of 8 days (as shown in FIG. 4A). No dose exhibited an antitumor effect (as described in the preceding section and shown in FIG. 2A, FIG. 2B, FIG. 2C, and FIG. 2D), but the highest dose caused splenomegaly. The effect of ketotifen on tumor stiffness in vivo was monitored using shear wave elastography (SWE). SWE measurements were performed before, after 3 days (for MCA205) or 4 days (for K7M2wt) of ketotifen treatment and upon study completion. The results demonstrated that the 10 mg/kg dose reduced tissue stiffness most effectively in MCA205 fibrosarcoma tumors, with elastic modulus values reaching 20 kPa (FIG. 4B). K7M2wt osteosarcoma tumors were similarly benefited from 10 mg/kg of daily ketotifen (FIG. 4C). The pertinent reduction in tumor stiffness was further validated ex vivo at the microscopic tissue scale using atomic force microscopy (AFM) of tumor samples. AFM analysis indicated a reduction in the average value of elastic modulus of K7M2wt tumors (FIG. 19A), with a shifted distribution towards lower values, which suggests a decrease in collagen density (FIG. 19B). In FIG. 19B, the left peak of lower elastic modulus corresponds to compliant cancer cells, while the tail of higher elastic modulus (dashed rectangles) represents the contribution of the stiffer matrix components, mainly collagen. Data are presented as mean±SE. Statistical analyses were performed by comparing means between two independent groups using the ordinary one-way ANOVA test.

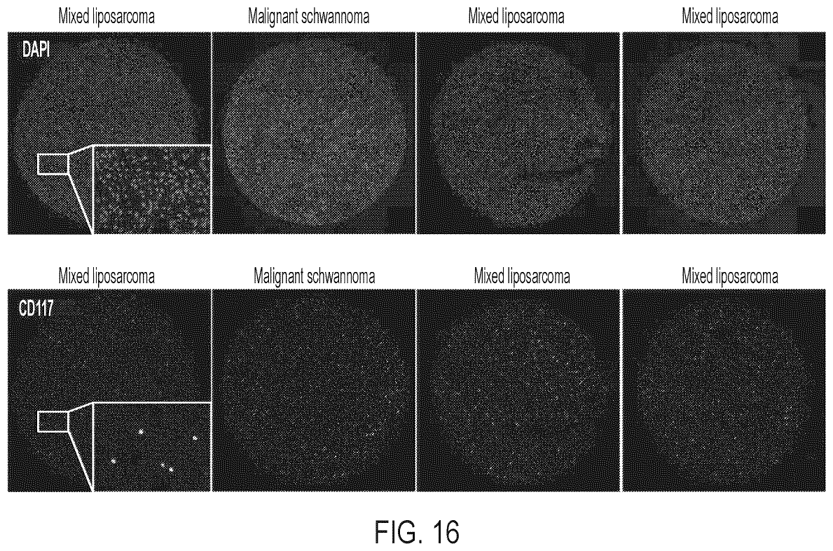

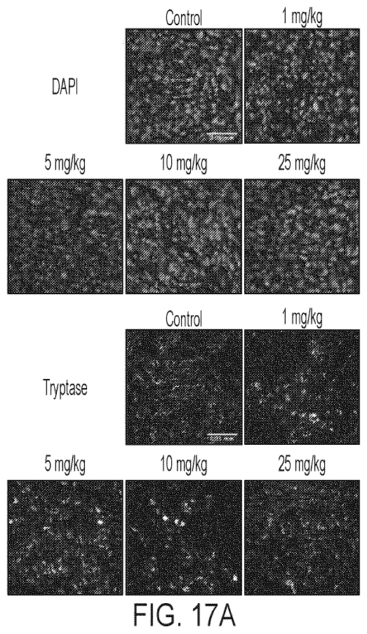

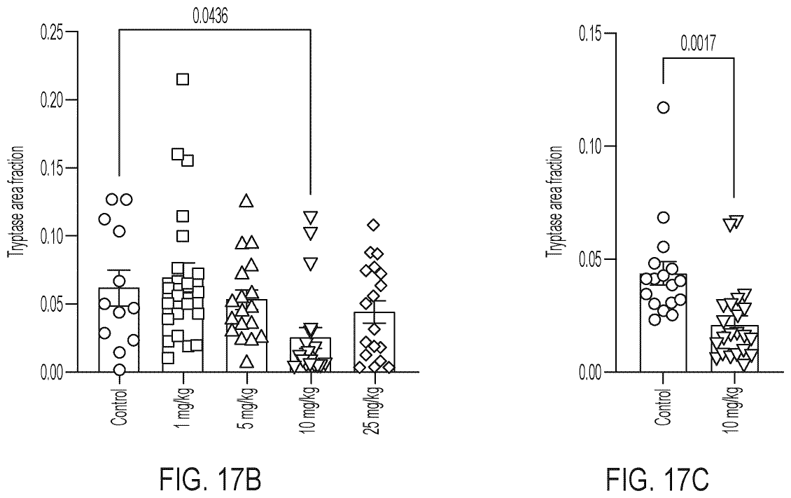

Finally, to confirm the effect of ketotifen on MC inactivation, ketotifen-treated tumors were stained for tryptase, a serine protease unique to MCs with a role in myofibroblast proliferation and collagen synthesis. The results show that tryptase levels were reduced after treatment with 10 mg/kg ketotifen in both sarcoma models, indicating that MCs influence collagen remodeling in the tumors (FIG. 17A, FIG. 17B, and FIG. 17C).

### Ketotifen Restores Perfusion and Normoxia by Vessel Normalization

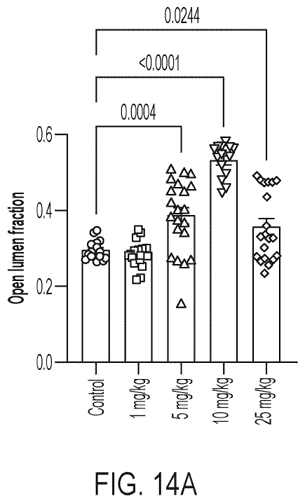

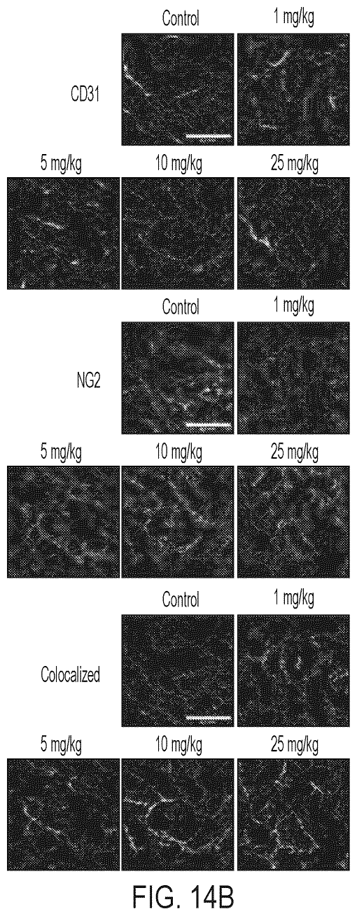

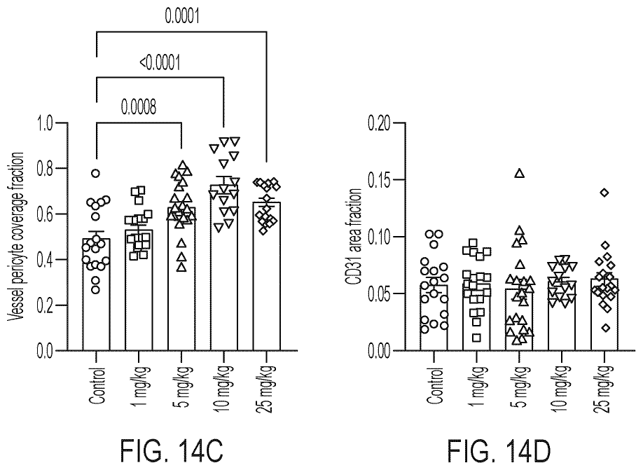

To assess whether MC inhibition could normalize the structure of tumor blood vessels, which can further improve perfusion, the effect of ketotifen on vascular permeability was assessed. Administration of ketotifen at 5, 10, and 25 mg/kg was found to increase the pericyte coverage of the tumor vasculature that fortifies the vessel walls, as indicated by the colocalization of the NG2 pericyte marker and CD31 endothelial protein, compared to the lowest dose and untreated control (FIG. 14B, FIG. 14C, and FIG. 14D). Surprisingly, a reduction in VEGF transcripts was not observed. Instead, an upregulation of IFN-γ transcription levels in MCA205 tumors following 10 mg/kg ketotifen treatment was observed (FIG. 1D). IFNγ is an angiostatic protein whose concentration in the TME has been related to vessel normalization and reduced vessel leakiness. Furthermore, the reduction in IFP levels with ketotifen treatment provides further evidence of vascular normalization (see FIG. 1E, as discussed above, showing ketotifen's effect in reducing interstitial fluid pressure of tumors). Ketotifen was also found to increase the fraction of vessels with open lumen as a result of vessel decompression (FIG. 14A), further indicating that ketotifen can decompress tumor vessels and improve perfusion.

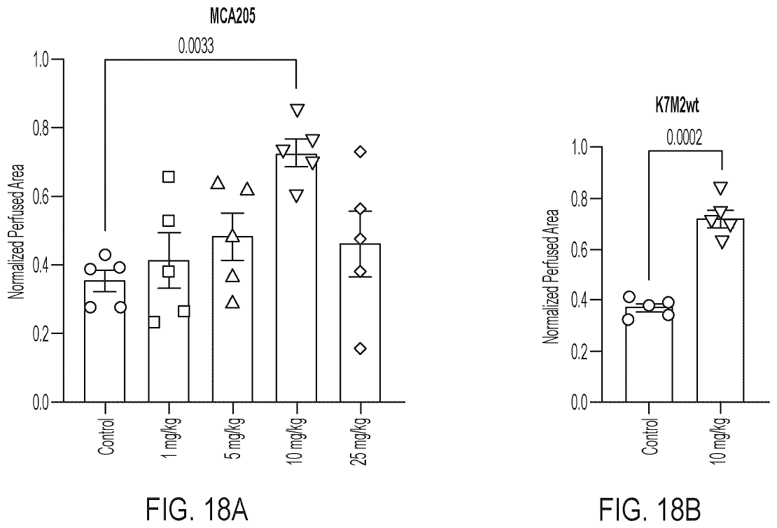

Whether such vessel decompression and normalization was sufficient to reestablish tumor blood perfusion was assessed. Using contrast enhanced ultrasound imagining, a significant increase in the area of the tumor covered by the contrast agent was found as a result of ketotifen treatment as compared to control-treated tumors. The findings indicated that 10 mg/kg ketotifen was highly effective at increasing vessel perfusion in both fibrosarcoma and osteosarcoma tumors (FIG. 18A and FIG. 18B). Similarly, by measuring the intratumoral levels of hypoxia using pimonidazole, it was observed that 10 mg/kg ketotifen alleviated hypoxia in both tumor models (FIG. 1A, FIG. 1B, and FIG. 1C), as discussed above.

Accordingly, it was found that ketotifen treatment, especially a daily dose of 10 mg/kg ketotifen, effectively reprograms mast cells and cancer-associated fibroblasts, normalizing both the tumor ECM and vasculature, improving perfusion and oxygenation.

In conclusion, ketotifen was demonstrated to have reprogramming properties which can be combined with the cytotoxic activities of doxorubicin and/or anti-PD-L1 therapy. Pretreatment with ketotifen increased the sensitivity of MCA205 and K7M2wt tumors to chemotherapy and checkpoint inhibition, while the combination of the three augmented antitumor response as indicated by the regression of tumor volume. Doxorubicin synergized with immunotherapy to promote CD8+ T cell proliferation and T cell attachment to vessels, independently of hypoxia alleviation, suggesting that other mechanisms may underline such antitumor responses. These findings indicate an IFNγ-dependent mechanism by which ketotifen facilitates vascular normalization and in turn upgregulates adhesion molecules necessary to support T cell extravasation, increase recruitment, and double the fraction of CD8+ T cells proliferating in these tumors, while decreasing the polarization of immature myeloid cell population into MDSCs. Finally, shear wave elastography was shown as a non-invasive and reliable biomarker of responsiveness to immune checkpoint inhibition by monitoring of tissue elastic properties. Tumor elastic properties correlated well with the efficacy of immunotherapy and chemotherapy in sarcoma tumors, highlighting the use of tumor stiffness to serve as a predictive marker of response.

What is claimed is:

1. A method for treating a solid tumor in a subject in need thereof comprising administering to the subject an effective amount of ketotifen, or a pharmaceutically acceptable salt thereof, in combination with a checkpoint inhibitor.

2. A method for initiating, enhancing or prolonging the effects of a checkpoint inhibitor, or enabling a subject to respond to a checkpoint inhibitor in a subject in need thereof comprising administering to the subject an effective amount of ketotifen, or a pharmaceutically acceptable salt thereof, in combination with a checkpoint inhibitor, wherein the subject has a solid tumor.

3. A method for potentiating the effects of a checkpoint inhibitor in a subject in need thereof comprising administering to the subject an effective amount of ketotifen, or a pharmaceutically acceptable salt thereof, in combination with a checkpoint inhibitor, wherein the subject has a solid tumor.

4. A method of increasing blood flow of a solid tumor in a subject comprising administering to the subject an effective amount of ketotifen, or a pharmaceutically acceptable salt thereof, in combination with a checkpoint inhibitor, wherein increasing blood flow of the solid tumor enhances the effect of the checkpoint inhibitor.

5. The method of claim 4, wherein blood flow is measured using ultrasound-based blood flow measurements or using histological techniques to measure hypoxia.

6. A method of improving the delivery or efficacy of a checkpoint inhibitor in a subject comprising administering an effective amount of ketotifen, or a pharmaceutically acceptable salt thereof, in combination with the checkpoint inhibitor, wherein the subject has a solid tumor, thereby improving the delivery or efficacy of the therapy in the subject.

7. The method of any one of claims 1-6, wherein administering ketotifen, or pharmaceutically acceptable salt thereof, increases the number of anti-tumor T cells that colocalize with the solid tumor.

8. The method of any one of claims 1-7, wherein administering ketotifen, or pharmaceutically acceptable salt thereof, reduces the tissue stiffness of the solid tumor.

9. The method of claim 8, wherein the tissue stiffness of the solid tumor is measured using ultrasound elastography.

10. The method of any one of claims 1-9, wherein administering ketotifen, or pharmaceutically acceptable salt thereof, decreases the levels of an extracellular matrix protein in the solid tumor.

11. The method of claim 10, wherein the extracellular matrix protein is collagen I or hyaluronan binding protein (HABP).

12. The method of any one of claims 1-11, wherein administering ketotifen, or pharmaceutically acceptable salt thereof, reduces hypoxia in the solid tumor.

13. The method of any one of claims 1-12, wherein the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof.

14. The method of claim 13, wherein the checkpoint inhibitor is a CTLA-4, PD-L1, PD-L2, or PD-1 inhibitor.

15. The method of claim 13, wherein the checkpoint inhibitor is an anti-PD-1 antibody, anti-PD-L1 antibody, or anti-CTLA4 antibody.

16. The method of claim 13, wherein the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559.

17. The method of any one of claim 13-16, wherein the checkpoint inhibitor is a combination of an anti-PD-1 antibody and an anti-CTLA-4 antibody.

18. The method of any one of claims 1-17, wherein the ketotifen, or pharmaceutically acceptable salt thereof, is administered to the subject once per day.

19. The method of any one of claims 1-17, wherein the ketotifen, or pharmaceutically acceptable salt thereof, is administered to the subject twice per day.

20. The method of any one of claims 1-19, wherein the ketotifen, or pharmaceutically acceptable salt thereof, is administered to the subject at a dose from about 0.01 mg/kg to about 5 mg/kg.

21. The method of any one of claims 1-19, wherein the ketotifen, or pharmaceutically acceptable salt thereof, is administered to the subject at a dose from about 100 mg to about 1200 mg.

22. The method of any one of claims 1-19, wherein the ketotifen, or pharmaceutically acceptable salt thereof, is administered to the subject at a dose from about 125 mg to about 500 mg.

23. The method of any one of claims 1-19, wherein the ketotifen, or pharmaceutically acceptable salt thereof, is administered to the subject at a dose of about 125 mg.

24. The method of any one of claims 1-19, wherein the ketotifen, or pharmaceutically acceptable salt thereof, is administered to the subject at a dose of about 500 mg.

25. The method of any one of claims 1-24, wherein the ketotifen, or pharmaceutically acceptable salt thereof, is administered to the subject prior to the subject being administered the checkpoint inhibitor.

26. The method of claim 25, wherein the ketotifen, or pharmaceutically acceptable salt thereof, is administered to the subject beginning at least 1 day prior to the subject being administered the checkpoint inhibitor.

27. The method of claim 25, wherein the ketotifen, or pharmaceutically acceptable salt thereof, is administered to the subject beginning at least 2 days prior to the subject being administered the checkpoint inhibitor.

28. The method of claim 25, wherein the ketotifen, or pharmaceutically acceptable salt thereof, is administered to the subject beginning at least 3 days prior to the subject being administered the checkpoint inhibitor.

29. The method of claim 25, wherein the ketotifen, or pharmaceutically acceptable salt thereof, is administered to the subject beginning at least 5 days prior to the subject being administered the checkpoint inhibitor.

30. The method of any one of claims 1-29, wherein the administration of ketotifen, or pharmaceutically acceptable salt thereof, to the subject is maintained for at least a portion of the time the subject is administered the checkpoint inhibitor.

31. The method of claim 30, wherein the administration of ketotifen, or pharmaceutically acceptable salt thereof, to the subject is maintained for the entire period of time the subject is administered the checkpoint inhibitor.

32. The method of any one of claims 1-31, wherein one or more therapeutic effects in the subject is improved after administration of the ketotifen, or pharmaceutically acceptable salt thereof, and the checkpoint inhibitor relative to a baseline.

33. The method of claim 32, wherein the one or more therapeutic effects is selected from the group consisting of: size of a tumor derived from the cancer, objective response rate, duration of response, time to response, progression free survival and overall survival.

34. The method of any one of claims 1-33, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the ketotifen, or pharmaceutically acceptable salt thereof, and the checkpoint inhibitor.

35. The method of any one of claims 1-34, wherein the objective response rate is at least about 20%, at least about 25%, at least about 30%, at least about 35%, at least about 40%, at least about 45%, at least about 50%, at least about 60%, at least about 70%, or at least about 80%.

36. The method of any one of claims 1-35, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor.

37. The method of any one of claims 1-36, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor.

38. The method of any one of claims 1-37, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the ketotifen, or pharmaceutically acceptable salt thereof, and the checkpoint inhibitor.

39. The method of any one of claims 1-38, wherein the solid tumor is selected from the group consisting of mesothelioma, breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma.

40. The method of claim 39, wherein the solid tumor is a sarcoma.

41. The method of claim 40, wherein the sarcoma is osteosarcoma or fibrosarcoma.

42. The method of any one of claims 1-41, wherein the subject is a human.

43. The method of any one of claims 1-42, wherein the method further comprises administering an additional chemotherapeutic agent.

44. The method of claim 43, wherein the additional chemotherapeutic agent is doxorubicin or an analogue or derivative thereof.

45. A kit comprising:
(a) an effective amount of ketotifen, or a pharmaceutically acceptable salt thereof;
(b) an effective amount of a checkpoint inhibitor; and
(c) instructions for using the ketotifen, or a pharmaceutically acceptable salt thereof and the checkpoint inhibitor according to the method of any one of claims 1-44.

46. A method of determining an effective amount of ketotifen in a subject with a solid tumor comprising:
(a) measuring the blood flow and/or stiffness of the solid tumor;
(b) administering to the subject an effective amount of ketotifen; and
(c) measuring the blood flow and/or stiffness of the solid tumor after administering the ketotifen, wherein an increase in blood flow and/or a decrease in stiffness following administration of the ketotifen to the subject indicates that the amount administered was an effective amount.

47. A method for treating a solid tumor in a subject in need thereof comprising:
(a) measuring the blood flow and/or stiffness of the solid tumor;
(b) administering to the subject an effective amount of ketotifen;
(c) measuring the blood flow and/or stiffness of the solid tumor after administering the ketotifen; and
(d) administering a chemotherapeutic agent if the blood flow of the solid tumor is increased and/or the stiffness of the solid tumor is decreased after administering the ketotifen.

48. A method for treating a solid tumor in a subject in need thereof comprising:
(a) measuring the blood flow and/or stiffness of the solid tumor;
(b) administering to the subject an effective amount of ketotifen;
(c) measuring the blood flow and/or stiffness of the solid tumor after administering the ketotifen;
(d) determining that the subject is responsive to a chemotherapeutic agent based on an increase in the blood flow of the solid tumor or a decrease in the stiffness of the solid tumor after administering the ketotifen; and
(e) administering the chemotherapeutic agent to the subject who has been determined to be responsive to the chemotherapeutic agent based on the increase in the blood flow of the solid tumor or the decrease in the stiffness of the solid tumor after administering the ketotifen.

49. A method for predicting response to treatment with a chemotherapeutic agent comprising:
(a) measuring the blood flow and/or stiffness of the solid tumor;
(b) administering to the subject an effective amount of ketotifen;
(c) measuring the blood flow and/or stiffness of the solid tumor after administering the ketotifen,
wherein an increase in the blood flow of the solid tumor or a decrease in the stiffness of the solid tumor after administering the ketotifen indicates that the subject is likely to respond to treatment with the chemotherapeutic agent.

50. The method of any one of claims 45-47, wherein the effective amount of ketotifen is determined by measuring the change in blood flow and/or stiffness of the solid tumor following administration of the ketotifen to the subject, wherein an increase in blood flow and/or a decrease in stiffness following administration of the ketotifen to the subject indicates that the amount administered was an effective amount.

51. The method of any one of claims 46-50, wherein the method comprises measuring the blood flow of the solid tumor and the blood flow of the solid tumor is increased after administering the ketotifen.

52. The method of any one of claims 46-50, wherein the method comprises measuring the stiffness of the solid tumor and the stiffness of the solid tumor is decreased after administering the ketotifen.

53. The method of any one of claims 46-52, wherein the ketotifen is administered for at least 1 day, at least 2 days, at least 3 days, at least 4 days, or at least 5 days prior to the administration of the chemotherapeutic agent.

54. The method of any one of claims 46-53, wherein the ketotifen is administered at a dose that increases the blood flow of the solid tumor and/or decreases the stiffness of the solid tumor.

55. The method of any one of claims 46-54, wherein blood flow and/or stiffness of the solid tumor is measured using ultrasound.

56. The method of claim any one of claims 46-55, wherein blood flow of the solid tumor is measured using histological techniques to measure hypoxia.

57. The method of any one of claims 47-56, wherein the chemotherapeutic agent is a checkpoint inhibitor.

58. The method of claim 57, wherein the checkpoint inhibitor inhibits a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN-15049, CHK1, CHK2, A2aR, and B-7 family ligands or a combination thereof.

59. The method of claim 58, wherein the checkpoint inhibitor is a CTLA-4, PD-L1, PD-L2, or PD-1 inhibitor.

60. The method of claim 58, wherein the checkpoint inhibitor is an anti-PD-1 antibody, anti-PD-L1 antibody, or anti-CTLA4 antibody.

61. The method of claim 58, wherein the checkpoint inhibitor is selected from the group consisting of MEDI0680, AMP-224, nivolumab, pembrolizumab, pidilizumab, MEDI4736, atezolizumab, ipilimumab, tremelimumab, and BMS-936559.

62. The method of any one of claim 58-61, wherein the checkpoint inhibitor is a combination of an anti-PD-1 antibody and an anti-CTLA-4 antibody.

63. The method of any one of claims 46-62, wherein the solid tumor is selected from the group consisting of breast cancer, breast cancer lung metastases, sarcoma, pancreatic cancer, ovarian cancer, liver metastases, prostate cancer, brain cancer, melanoma, renal cell carcinoma, colorectal cancer, hepatocellular carcinoma, lung cancer, squamous cell carcinoma of the head and neck, urothelial carcinoma, esophageal squamous cell carcinoma, gastric cancer, esophageal cancer, cervical cancer, Merkel cell carcinoma, endometrial carcinoma, and cutaneous squamous cell carcinoma.

64. The method of claim 63, wherein the solid tumor is a sarcoma.

65. The method of claim 64, wherein the sarcoma is osteosarcoma or fibrosarcoma.

66. The method of any one of claims 46-65, wherein the subject is a human.

67. The method of any one of claims 47-56 or 63-66, wherein the chemotherapeutic agent is doxorubicin.


SEQUENCE LISTING (26 sequences)

SEQ ID NO: 1 - DNA, 21 bp, synthetic construct
  source 1..21: mol_type = other DNA
atgaacgctacacactgcatc

SEQ ID NO: 2 - DNA, 21 bp, synthetic construct
  source 1..21: mol_type = other DNA
ccatccttttgccagttcctc

SEQ ID NO: 3 - DNA, 20 bp, synthetic construct
  source 1..20: mol_type = other DNA
agcacagcagatgtgaatgc

SEQ ID NO: 4 - DNA, 20 bp, synthetic construct
  source 1..20: mol_type = other DNA
tttcttgcgctttcgttttt

SEQ ID NO: 5 - DNA, 20 bp, synthetic construct
  source 1..20: mol_type = other DNA
gacggccaggtcatcactat

SEQ ID NO: 6 - DNA, 20 bp, synthetic construct
  source 1..20: mol_type = other DNA
aaggaaggctggaaaagagc

SEQ ID NO: 7 - DNA, 20 bp, synthetic construct
  source 1..20: mol_type = other DNA
gagcggagagtactggatcg

SEQ ID NO: 8 - DNA, 20 bp, synthetic construct
  source 1..20: mol_type = other DNA
gttcgggctgatgtaccagt

SEQ ID NO: 9 - DNA, 19 bp, synthetic construct
  source 1..19: mol_type = other DNA
cactctccagtggagttca

SEQ ID NO: 10 - DNA, 20 bp, synthetic construct
  source 1..20: mol_type = other DNA
gtaatggcaggcacaggtct

SEQ ID NO: 11 - DNA, 20 bp, synthetic construct
  source 1..20: mol_type = other DNA
tgtgctggactctggagatg

SEQ ID NO: 12 - DNA, 20 bp, synthetic construct
  source 1..20: mol_type = other DNA
gaaggaatagccacgctcag

SEQ ID NO: 13 - DNA, 20 bp, synthetic construct
  source 1..20: mol_type = other DNA
ataagcggtcctctgggaat

SEQ ID NO: 14 - DNA, 20 bp, synthetic construct
  source 1..20: mol_type = other DNA
cctgttggtaaggtgcctgt

SEQ ID NO: 15 - DNA, 20 bp, synthetic construct
  source 1..20: mol_type = other DNA
ttccaaacctcaaggtggtc

SEQ ID NO: 16 - DNA, 20 bp, synthetic construct
  source 1..20: mol_type = other DNA
tgctacgccacacaaagaag

SEQ ID NO: 17 - DNA, 20 bp, synthetic construct
  source 1..20: mol_type = other DNA
gacggccaggtcatcactat

SEQ ID NO: 18 - DNA, 20 bp, synthetic construct
  source 1..20: mol_type = other DNA
aaggaaggctggaaaagagc

SEQ ID NO: 19 - DNA, 20 bp, synthetic construct
  source 1..20: mol_type = other DNA
cctgtttcctgcctctgaag

SEQ ID NO: 20 - DNA, 20 bp, synthetic construct
  source 1..20: mol_type = other DNA
ttaaggtcctctgcgtctcc

SEQ ID NO: 21 - DNA, 20 bp, synthetic construct
  source 1..20: mol_type = other DNA
cccaggtggaggtctactca

SEQ ID NO: 22 - DNA, 20 bp, synthetic construct
  source 1..20: mol_type = other DNA
caggattttgggagctggta

SEQ ID NO: 23 - DNA, 20 bp, synthetic construct
  source 1..20: mol_type = other DNA
gtccacggagagtttggtgt

SEQ ID NO: 24 - DNA, 20 bp, synthetic construct
  source 1..20: mol_type = other DNA
aagtggtgttcggaccaaag

SEQ ID NO: 25 - DNA, 20 bp, synthetic construct
  source 1..20: mol_type = other DNA
gacggccaggtcatcactat

SEQ ID NO: 26 - DNA, 20 bp, synthetic construct
  source 1..20: mol_type = other DNA
aaggaaggctggaaaagagc